(19) United States
(12) Patent Application Publication
(10) Pub. No.: US 2020/0332306 A1
(43) Pub. Date: Oct. 22, 2020

(54) TYPE I-E CRISPR-CAS SYSTEMS FOR EUKARYOTIC GENOME EDITING

(71) Applicant: PIONEER HI-BRED INTERNATIONAL, INC., JOHNSTON, IA (US)

(72) Inventors: STEPHEN LAWRENCE GASIOR, JOHNSTON, IA (US); JOSHUA K YOUNG, JOHNSTON, IA (US)

(73) Assignee: PIONEER HI-BRED INTERNATIONAL, INC., JOHNSTON, IA (US)

(21) Appl. No.: 16/754,090

(22) PCT Filed: Oct. 8, 2018
(86) PCT No.: PCT/US18/54856

§ 371 Date: Apr. 6, 2020

Related U.S. Application Data
(60) Provisional application No. 62/569,836, filed on Oct. 9, 2017.
(60) Provisional application No. 62/639,791, filed on Mar. 7, 2018.
(60) Provisional application No. 62/670,434, filed on May 11, 2018.
(60) Provisional application No. 62/693,533, filed on Jul. 3, 2018.

(51) Int. Cl. C12N 15/82; C12N 9/22

(52) U.S. Cl. CPC C12N 15/8213; C12N 2310/20; C12N 9/22; C12N 15/8205

(57) ABSTRACT

Compositions and methods are provided for modification of a target sequence in the genome of a cell. The methods and compositions employ a cascade system and guide polynucleotide to provide an effective system for targeting, binding to, and modifying or altering target sequences within the genome of a cell or organism. Also provided are systems further comprising an endonuclease. Compositions and methods are also provided for guide polynucleotide/endonuclease systems comprising at least one nuclease covalently or non-covalently linked to, or assembled with, at least one protein subunit of a cascade, and for compositions and methods for direct delivery of endonucleases.

### CROSS-REFERENCE TO RELATED APPLICATION

This application is a 371 National Stage application of PCT application number PCT/US2018/054856 filed on 8 Oct. 2018, which claims the benefit of U.S. Provisional Patent Application Ser. No. 62/569,836 filed 9 Oct. 2017, U.S. Provisional Patent Application Ser. No. 62/639,791 filed 7 Mar. 2018, U.S. Provisional Patent Application Ser. No. 62/670,434 filed 11 May 2018, and U.S. Provisional Patent Application Ser. No. 62/693,533 filed 3 Jul. 2018, all of which are herein incorporated by reference in their entireties.

### FIELD OF THE INVENTION

The disclosure relates to the field of molecular biology, in particular to compositions of guide polynucleotide/endonuclease systems, and compositions and methods for modifying the genome of a cell.

### REFERENCE TO SEQUENCE LISTING SUBMITTED ELECTRONICALLY

The official copy of the sequence listing is submitted electronically via EFS-Web as an ASCII formatted sequence listing with a file named 7468-US-PCT_SequenceListing_ST25.TXT created on 27 Mar. 2020 and having a size of 751,565 bytes and is filed concurrently with the specification. The sequence listing comprised in this ASCII formatted document is part of the specification and is herein incorporated by reference in its entirety.

### BACKGROUND

Recombinant DNA technology has made it possible to insert DNA sequences at targeted genomic locations and/or modify specific endogenous chromosomal sequences. Site-specific integration techniques, which employ site-specific recombination systems, as well as other types of recombination technologies, have been used to generate targeted insertions of genes of interest in a variety of organism. Genome-editing techniques such as designer zinc finger nucleases (ZFNs), transcription activator-like effector nucleases (TALENs), or homing meganucleases, are available for producing targeted genome perturbations, but these systems tend to have low specificity and employ designed nucleases that need to be redesigned for each target site, which renders them costly and time-consuming to prepare.

Newer technologies utilizing archaeal or bacterial adaptive immunity systems have been identified, called CRISPR (Clustered Regularly Interspaced Short Palindromic Repeats), which comprise different domains of effector proteins that encompass a variety of activities (DNA recognition, binding, and optionally cleavage).

Despite the identification and characterization of some of these systems, there remains a need for identifying novel effectors and systems, as well as demonstrating activity in eukaryotes, particularly animals and plants, to effect editing of endogenous and previously-introduced heterologous polynucleotides.

### SUMMARY

Methods and compositions are provided for the modification of a target polynucleotide sequence, for example in the genome of a cell, for example in the genome of a plant cell, through the usage of a Type I-E CRISPR Cascade system. In some aspects, a guide RNA directs one or more components to a target polynucleotide. The guide RNA may be expressed under control of a heterologous regulatory element, for example a polII or polIII promoter. Multiple guide RNAs may be expressed from a single promoter. The target polynucleotide may be modified by the insertion of one or more nucleotides, deletion of one or more nucleotides, substitution of one or more nucleotides, chemical modification of one or more nucleotides, or any combination of the preceding.

In some aspects, the target polynucleotide is a gene, that may be activated or deactivated as the result of the target site modification.

In some aspects, the target site polynucleotide modification may involve the usage of one, two, three, four, five, or more than five components of a Cascade. In some aspects, one or more components of the Cascade may be fused to another polypeptide, for example a nuclease or a transcriptional activator. In some aspects, a synergistic effect is achieved by fusing the same or different polypeptide domains to different components of the Cascade and/or the endonuclease.

In some aspects, targeted cleavage is achieved through the use of a Type I-E Cascade that is associated with a molecule capable of introducing a single-strand nick or a double-strand break to a polynucleotide sequence at a target site, such as Cas3 nuclease or Cas3 nickase or Fok1 or I-Tevi. A Ias3 nickase, when recruited by a Type I-E cleavage cascade, may nick either or both strands of a double-strand target, creating one nick on one strand or one nick on both strands, resulting in either a nick or a double-strand break. The double-strand break may comprise a 5′ overhang, a 3′ overhang, a blunt end cut, or a blunt end cut plus overhangs. In some aspects, the Cas3 nickase works in a cooperative manner, that is, the nickase nicks one strand of a double stranded polynucleotide and nicks the opposite strand of the same double stranded polynucleotide, to create a double-strand break, by virtue of the cooperative nicks on the two strands.

In one aspect, the invention provides a method for forming a CRISPR cascade and guide polynucleotide complex in a plant cell, comprising providing to said plant cell: a Cse1 polypeptide, and a guide polynucleotide comprising a variable targeting domain that is substantially complementary to a polynucleotide target sequence in the genome of the plant cell; and incubating said plant cell; wherein said Cse1 polypeptide functionally associates with at least one other protein to form a cascade; and wherein the guide polynucleotide and cascade form a complex capable of recognizing and binding to the polynucleotide target sequence in the genome of the plant cell.

In one aspect, the invention provides a method for forming a CRISPR cascade and guide polynucleotide complex in a plant cell, comprising providing to said plant cell: a cse1 polynucleotide, and a guide polynucleotide comprising a variable targeting domain that is substantially complementary to a polynucleotide target sequence in the genome of said plant cell; and incubating said cell; wherein said cse1 polynucleotide is operably linked to a noncoding regulatory element rendering it capable of expressing a Cse1 polypeptide, and wherein said Cse1 polypeptide functionally associates with at least one other protein to form a cascade; and wherein said cascade and guide polynucleotide form a complex capable of recognizing and binding to a polynucleotide target sequence in the genome of said plant cell.

In any aspect of any of the methods comprising Cas5, Cash, or Cas8, the invention optionally provides the Cas protein linked to a heterologous polypeptide, wherein said heterologous polypeptide is capable of a function selected from the group consisting of: transcription repression, transcription modulation, transcription activation, polynucleotide methylation, polynucleotide demethylation, polynucleotide cleavage, polynucleotide nicking, deamination, and a combination thereof.

In any aspect of any of the methods comprising Cas5, Cash, or Cas8, the invention optionally provides the Cas protein linked to a heterologous polypeptide, wherein said Cas protein and said heterologous polypeptide are linked at the C-terminal end of the Cas protein. In some aspects, the Cas5 and the heterologous polypeptide are linked by 1, 2, 3, 4, 5, 6, 7, 8, 9, 10, 11, 12, 13, 14, 15, 16, 17, 18, 19, 20, 21, 22, 23, 24, 25, or greater than 25 amino acids.

In some aspects, the Cascade and guide polynucleotide form a complex capable of recognizing and binding to a polynucleotide target sequence in the genome of said plant cell; wherein one, two, three, or four polynucleotide(s) selected from the group consisting of: cse2, cas7, cas5, and cash is(are) further introduced into said plant cell. In some aspects, the complex may nick, cleave, or edit at least one nucleotide of the target polynucleotide.

In some aspects, the introduction of any component into the target cell is via a method selected from the group consisting of: Agrobacterium-mediated transformation, particle bombardment, and floral dip.

In some aspects, the plant cell is incubated at a temperature of 28 degrees Celsius, between 28 and 29, 29, between 29 and 30, 30, between 30 and 31, 31, between 31 and 32, 32, between 32 and 33, 33, between 33 and 34, 34, between 34 and 35, 35, between 35 and 36, 36, between 36 and 37, 37, or greater than 37 degrees Celsius, for at least 0.5, 1, 2, 3, 4, or greater than 4 hours.

In some aspects, the plant cell further comprises a guide polynucleotide comprising a sequence substantially complementary to a sequence within the genome of said plant cell.

In some aspects, the said plant cell comprises an endonuclease. In some aspects, said endonuclease is Cas3, or a functional fragment or variant thereof. In some aspects, said endonuclease is a fusion protein comprising at least one functional endonuclease domain.

In some aspects, the method or composition further comprising at least one of the components provided to said plant cell comprising the capability of nicking or cleaving at least one strand of the polynucleotide target sequence. In some aspects, the method or composition further comprises editing at least one base of the polynucleotide target sequence.

In some aspects, said plant cell is a monocot plant cell. In some aspects, said monocot plant cell is obtained or derived from a plant selected from the group consisting of: corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

In some aspects, said plant cell is a dicot plant cell. In some aspects, said dicot plant cell is obtained or derived from a plant selected from the group consisting of: soybean (Glycine max), Brassica species (for example but not limited to: oilseed rape or Canola) (Brassica napus, B. campestris, Brassica rapa, Brassica juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), and potato (Solanum tuberosum).

In some aspects, the method further comprises placing the plant cell in a medium that promotes growth, generating a plant from the plant cell, and screening said plant for a trait of interest;

In some aspects, the said Cse1 polypeptide or cse1 polynucleotide is derived from a host organism comprising a Type I-E CRISPR system.

In some aspects, the cse1 polynucleotide is operably linked to a noncoding regulatory element rendering it capable of expressing a Cse1 polypeptide that functionally associates with at least one other protein to form a cascade

In some aspects, the said cascade further comprises at least one, at least two, at least three, or four protein(s) selected from the group consisting of: Cse2, Cas7, Cas5, and Cas6.

In some aspects, the Cas5, Cas6, or Cas8 is linked to a heterologous polypeptide, wherein said heterologous polypeptide is capable of a function selected from the group consisting of: transcription repression, transcription modulation, transcription activation, polynucleotide methylation, polynucleotide demethylation, polynucleotide cleavage, polynucleotide nicking, deamination, and a combination thereof.

In some aspects, the Cas5, Cas6, or Cas8 is linked to a heterologous polypeptide, wherein said heterologous polypeptide is capable of a function selected from the group consisting of: transcription repression, transcription modulation, transcription activation, polynucleotide methylation, polynucleotide demethylation, polynucleotide cleavage, polynucleotide nicking, deamination, and a combination thereof; wherein the heterologous polynucleotide is linked to the C-terminal end of the Cas protein, optionally via a linker of at least 1, 2, 3, 4, 5, 6, 7, 8, 9 10, 11, 12, 13, 14, 15, 16, 17, 18, 19, 20, 21, 22, 23, 24, 25, or greater than 25 amino acids.

In some aspects, the at least one, two three, or four additional polynucleotides is further introduced into said plant cell, said additional polynucleotides selected from the group consisting of: cse2, cas7, cas5, and cas6.

In some aspects, at least one of the components is introduced into said plant cell via a recombinant DNA construct.

In some aspects, at least one of the components has been optimized for expression in said plant cell.

In some aspects, the introduction into said plant cell is achieved via a method selected from the group consisting of: Agrobacterium-mediated transformation, particle bombardment, and floral dip.

In some aspects, the plant cell is incubated at a temperature of greater than 25, 26, 27, 28, 29, 30, 31, 32, 33, 34, 35, or greater than 35 degrees Celsius.

In some aspects, the plant cell comprises an endonuclease. In some aspects, said endonuclease is Cas3, or a functional fragment or variant thereof In some aspects, said endonuclease is a fusion protein comprising at least one functional endonuclease domain.

In one aspect, the invention provides a whole organism, an organism part, an organism element, a tissue, a plurality of cells, a single cell, or a composition derived from one of the following: (a) a synthetic composition comprising: a plant cell, a guide polynucleotide comprising a variable targeting domain that is substantially complementary to a polynucleotide target sequence in the genome of said plant cell, an endonuclease, and a cascade comprising a Cse1 polypeptide; wherein said cascade forms a complex with the guide polynucleotide and the endonuclease; and wherein the complex is capable of recognizing, binding to, and nicking or cleaving at least one strand of a polynucleotide target sequence in the genome of said plant cell; or (b) a synthetic composition comprising: a plant cell, a guide polynucleotide comprising a variable targeting domain that is substantially complementary to a polynucleotide target sequence in the genome of said plant cell, an endonuclease, and a cse1 polynucleotide; wherein said cse1 polynucleotide is operably linked to a noncoding regulatory element rendering it capable of expressing a Cse1 polypeptide that functionally associates with at least one other protein to form a cascade; wherein said cascade forms a complex with the guide polynucleotide and the endonuclease; and wherein the complex is capable of recognizing, binding to, and nicking or cleaving at least one strand of a polynucleotide target sequence in the genome of said plant cell.

In one aspect, the invention provides an organism, progeny, or tissue derived from one of the following: (a) a synthetic composition comprising: a plant cell, a guide polynucleotide comprising a variable targeting domain that is substantially complementary to a polynucleotide target sequence in the genome of said plant cell, an endonuclease, and a cascade comprising a Cse1 polypeptide; wherein said cascade forms a complex with the guide polynucleotide and the endonuclease; and wherein the complex is capable of recognizing, binding to, and nicking or cleaving at least one strand of a polynucleotide target sequence in the genome of said plant cell; or (b) a synthetic composition comprising: a plant cell, a guide polynucleotide comprising a variable targeting domain that is substantially complementary to a polynucleotide target sequence in the genome of said plant cell, an endonuclease, and a cse1 polynucleotide; wherein said cse1 polynucleotide is operably linked to a noncoding regulatory element rendering it capable of expressing a Cse1 polypeptide that functionally associates with at least one other protein to form a cascade; wherein said cascade forms a complex with the guide polynucleotide and the endonuclease; and wherein the complex is capable of recognizing, binding to, and nicking or cleaving at least one strand of a polynucleotide target sequence in the genome of said plant cell.

In one aspect, the invention provides any of the following synthetic compositions: (a) a synthetic composition comprising: a plant cell, a guide polynucleotide comprising a variable targeting domain that is substantially complementary to a polynucleotide target sequence in the genome of said plant cell, an endonuclease, and a cascade comprising a Cse1 polypeptide; wherein said cascade forms a complex with the guide polynucleotide and the endonuclease; and wherein the complex is capable of recognizing, binding to, and nicking or cleaving at least one strand of a polynucleotide target sequence in the genome of said plant cell; or (b) a synthetic composition comprising: a plant cell, a guide polynucleotide comprising a variable targeting domain that is substantially complementary to a polynucleotide target sequence in the genome of said plant cell, an endonuclease, and a cse1 polynucleotide; wherein said cse1 polynucleotide is operably linked to a noncoding regulatory element rendering it capable of expressing a Cse1 polypeptide that functionally associates with at least one other protein to form a cascade; wherein said cascade forms a complex with the guide polynucleotide and the endonuclease; and wherein the complex is capable of recognizing, binding to, and nicking or cleaving at least one strand of a polynucleotide target sequence in the genome of said plant cell; wherein said cascade is a Type I-E CRISPR cascade.

In one aspect, the invention provides a synthetic composition comprising a Cas5 protein and a heterologous polypeptide, wherein said heterologous polypeptide is capable of a function selected from the group consisting of: transcription repression, transcription modulation, transcription activation, polynucleotide methylation, polynucleotide demethylation, polynucleotide cleavage, polynucleotide nicking, deamination, and a combination thereof.

In one aspect, the invention provides a synthetic composition comprising a Cas5 protein and a heterologous polypeptide, wherein said heterologous polypeptide is capable of a function selected from the group consisting of: transcription repression, transcription modulation, transcription activation, polynucleotide methylation, polynucleotide demethylation, polynucleotide cleavage, polynucleotide nicking, deamination, and a combination thereof; wherein said Cas5 protein and said heterologous polynucleotide are linked at the 5-prime end of Cas 5.

In any aspect of the invention, Cas5 is a polypeptide sequence shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:10. In any aspect, Cas5 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:2.

In any aspect of the invention, Cas6 is a polypeptide sequence shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:11. In any aspect, Cas6 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:3.

In any aspect of the invention, Cas7 is a polypeptide sequence shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:12. In any aspect, Cas7 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:4.

In any aspect of the invention, Cse1 is a polypeptide sequence shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:13. In any aspect, Cse1 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:5.

In any aspect of the invention, Cse2 is a polypeptide sequence shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:14. In any aspect, Cse2 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:6.

In any aspect of the invention, Cas1 is a polypeptide sequence shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:16. In any aspect, Cas1 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:8.

In any aspect of the invention, Cas2 is a polypeptide sequence shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:17. In any aspect, Cas2 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:9.

In any aspect of the invention, Cas3 is a polypeptide sequence shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:15. In any aspect, Cas3 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:7.

In any aspect, any component may be provided to the plant cell as a polynucleotide or a polypeptide. In some aspects, a combination of polynucleotide and polypeptide components may be provided.

### BRIEF DESCRIPTION OF THE DRAWINGS AND THE SEQUENCES

The disclosure can be more fully understood from the following detailed description and the accompanying drawings and Sequence Listing, which form a part of this application. The sequence descriptions and sequence listing attached hereto comply with the rules governing nucleotide and amino acid sequence disclosures in patent applications as set forth in 37 C.F.R. §§1.821 and 1.825. The sequence descriptions comprise the three letter codes for amino acids as defined in 37 C.F.R. §§ 1.821 and 1.825, which are incorporated herein by reference.

FIG. 1 depicts the locus architecture and operon structure of a typical Type I-E CRISPR-Cas system, comprising genes encoding Cas3, Cse1, Cse2, Cas7, Cas5, Cas6, Cas1, and Cas2, followed by a CRISPR array comprising repeats and spacers.

FIG. 2 depicts the components of a recombinant expression cassette comprising a cascade gen.

FIG. 3A depicts the cas5 gene linked at its 5′ end to a Nuclear Localization Sequence (NLS) and an activation motif (AtCBF1). FIG. 3B depicts the cas5 gene linked at its 3′ end to a Nuclear Localization Sequence (NLS) and an activation motif (AtCBF1).

FIG. 4 depicts the cse1 gene linked at its 5′ end to a Nuclear Localization Sequence (NLS) and an activation motif (AtCBF1).

FIG. 5A depicts the components of a recombinant expression cassette comprising a Cas gene (cas5 or cse1) fused at its 5-prime end via a linker sequence to an AtCBF1 transcriptional activator, operably linked to a maize ubiquitin promoter, maize ubiquitin 5-prime UTR, and maize ubiquitin intron, and further comprising a terminator sequence.

FIG. 5B depicts the components of a recombinant expression cassette comprising a Cas gene (cas5 or dcas9) fused at its 3-prime end via a linker sequence to an AtCBF1 transcriptional activator, operably linked to a maize ubiquitin promoter, maize ubiquitin 5-prime UTR, and maize ubiquitin intron, and further comprising a terminator sequence.

FIG. 6 depicts primary CRISPR array transcript architecture, and cleavage by an endonuclease (as one example, Cas6 is shown in the figure), to generate small CRISPR RNAs (guide RNAs).

FIG. 7 depicts a CRISPR array-like arrangement of Variable targeting domains comprising maize sequences interspersed with repeat sequences, operably linked to a U6 PolIII promoter and a U6 PolIII terminator.

FIG. 8 depicts a recombinant expression cassette comprising a maize UBI promoter, maize UBI 5-prime UTR, and maize UBI intron 1 operably linked to a CRISPR array, followed by a PolII terminator.

FIG. 9 depicts the key elements of a gene activation (GACT) DNA expression cassette, comprising a minimal promoter and 5-prime UTR operably linked to a gene encoding DS-Red Express, followed by a terminator.

FIG. 10 depicts fluorescence imaging of immature maize embryos (IMEs) transformed with the GACT cassette and: S. pyogenes deactivated Cas9 linked to the Arabidopsis CBF1 transcriptional activator via two different linkers GRA and 30XQ, in the absence of a guide RNA (negative controls), or S. pyogenes deactivated Cas9 linked to the Arabidopsis CBF1 transcriptional activator via two different linkers GRA and 30XQ in the presence of a guide RNA. The IMEs transformed with the S. pyogenes deactivated Cas9 linked to the Arabidopsis CBF1 transcriptional activator via the GRA linker showed greater fluorescence than the IMEs with the 30XQ linker.

FIG. 11 depicts fluorescence imaging of immature maize embryos (IMEs) simultaneously transformed with the GACT cassette and individual expression cassettes of the S. thermophilus Type I-E cascade proteins Cas5, Cse1, Cas6, Cas7, and Cse2, along with an expression cassette producing the guide RNA operably linked to either the PolII promoter or the PolIII promoter, as well as the GACT cassette; and incubated at either 28 degrees Celsius or 37 degrees Celsius. IMEs incubated at 37 degrees Celsius exhibited higher fluorescence than those at 28 degrees Celsius. IMEs expressing the guide RNA with the PolII promoter exhibited similar fluorescence as those expressing the guide RNA with the PolIII promoter.

FIG. 12 shows that Ds-Red gene activation was enhanced when AtCBF1 was linked to the C-termini of Cas6 and Cse1 relative to Cas5, and moreover, when combined, AtCBF1 linked Cas5, Cas6, and Cse1 Ds-Red gene activation surpassed that generated by Spy dCas9 linked (GRA) AtCBF1.Taken together, this indicates that the Type I-E cascade complex and guide RNA from Streptococcus thermophilus DGCC7710 may be engineered to form a functional complex in maize cells and be used for plant gene transcriptional activation.

FIG. 13 shows the endogenous R1 gene targeted for transcriptional up-regulation by cascade while over-expressing the C1 gene, to induce anthocyanin production using Sth7710 cascade.

FIG. 14 shows that the anthocyanin phenotype was observed for the Sth7710 cascade.

FIG. 15 shows cooperative nicking strategies using a Cas3 nickase and two Sth7710 cascade targets. The whole oval represents the cascade complex and the half oval depicts the Cas3 nickase recruited following cascade target recognition. Arrows illustrate strand specific DNA nicking by the Cas3 nickase.

FIG. 16 shows nicking using a Cas3 nickase and two Sth7710 cascade targets (A4+/A3−) (see Table 15) in close proximity (on opposite DNA strands) resulted in targeted chromosomal mutagenesis. The predominant mutation patterns were large deletions (>10 bp) of variable length that typically originated within one of the Sth7710 cascade target sequences although small deletion (1 or 2 bp) mutations were also detected. 3 additional pairs of Sth7710 cascade target sites (A2+/A1−, A2+/A3−, and A4+/A1−) (Table 15) were selected for testing with the Cas3 nickase.

FIG. 17A shows the gene encoding the Ds-Red Express fluorescent protein (SEQ ID NO:107) may be partially duplicated and a cascade target sequence can be incorporated between the duplicated fragments. To boost expression, the 5 prime end of the partially duplicated DsRed gene is operably linked to the cauliflower mosaic virus 35S (CAMV35S) enhancer (SEQ ID NO:127) and Maize Histone 2B (H2B) promoter (SEQ ID NO:192) and the 3 prime end fused with the Potato Proteinase Inhibitor II (PINII) terminator (SEQ ID NO:193) resulting in a Ds-target site-sRed reporter construct illustrated in FIG. 17A. With this configuration, cleavage and cellular repair of the target sequence will result in two outcomes. The first outcome is intramolecular DNA repair between the duplicated regions of the Ds-Red gene. This restores the gene resulting in Ds-Red expression and immunofluorescence (FIG. 17B). The second outcome (as shown in FIG. 17C) produces indel mutations indicative of DNA target cleavage and repair by the non-homologous end-joining (NHEJ) pathway that can be detected by targeted deep sequencing.

FIG. 18 shows that expression cassettes encoding I-TevI fused to the 5 prime end of the cse1 genes (SEQ ID NOS: 187 and 188) produced Ds-Red immunofluorescent signal, indicative of target cleavage and intramolecular repair and functional restoration of the Ds-Red gene.

FIG. 19 shows that deep sequencing of mutations at target site SEQ ID NO:194 yielded small insertion or deletion mutations originating within the cleavage sites of the I-Tev cleavage motif, CNNNG (where N represents any base pair A, T, G, or C), indicative of NHEJ DNA repair, further collaborating the fluorescent imaging data. Dashes represent deletions while lower case font denotes base pair insertions.

FIG. 20 shows that deep sequencing of mutations at target site SEQ ID NO:205 yielded small insertion or deletion mutations originating within the cleavage sites of the I-TevI cleavage motif, CNNNG (where N represents any base pair A, T, G, or C), indicative of NHEJ DNA repair further, collaborating the fluorescent imaging data. Dashes represent deletions while lower case font denotes base pair insertions.

FIG. 21 shows that mutations demonstrative of DNA target cleavage and NHEJ repair at target site SEQ ID NO:216 were also recovered for a DNA expression construct (SEQ ID NO: 185) where the I-TevI cleavage domain was appended to the 5 prime end of the cas6 gene. Dashes represent deletions while lower case font denotes base pair insertions.

FIG. 22 shows that mutations demonstrative of DNA target cleavage and NHEJ repair at target site SEQ ID NO:216 were also recovered for a DNA expression construct (SEQ ID NO: 186) where the I-TeVI cleavage domain was appended to the 5 prime end of the cas6 gene.

SEQ ID NO:1 is the Type I CRISPR repeat DNA sequence from Streptococcus thermophilus.

SEQ ID NO:2 is the cas5 gene DNA sequence from Streptococcus thermophilus.

SEQ ID NO:3 is the cas6 gene DNA sequence from Streptococcus thermophilus.

SEQ ID NO:4 is the cas7 gene DNA sequence from Streptococcus thermophilus.

SEQ ID NO:5 is the cse1 gene DNA sequence from Streptococcus thermophilus.

SEQ ID NO:6 is the cse2 gene DNA sequence from Streptococcus thermophilus.

SEQ ID NO:7 is the cas3 gene DNA sequence from Streptococcus thermophilus.

SEQ ID NO:8 is the cas1 gene DNA sequence from Streptococcus thermophilus.

SEQ ID NO:9 is the cas2 gene DNA sequence from Streptococcus thermophilus.

SEQ ID NO:10 is the Cas5 protein PRT sequence from Streptococcus thermophilus.

SEQ ID NO:11 is the Cas6 protein PRT sequence from Streptococcus thermophilus.

SEQ ID NO:12 is the Cas7 protein PRT sequence from Streptococcus thermophilus.

SEQ ID NO:13 is the Cse1 protein PRT sequence from Streptococcus thermophilus.

SEQ ID NO:14 is the Cse2 protein PRT sequence from Streptococcus thermophilus.

SEQ ID NO:15 is the Cas3 protein PRT sequence from Streptococcus thermophilus.

SEQ ID NO:16 is the Cas1 protein PRT sequence from Streptococcus thermophilus.

SEQ ID NO:17 is the Cas2 protein PRT sequence from Streptococcus thermophilus.

SEQ ID NO:18 is the SV40 NLS PRT sequence from Simian virus 40 .

SEQ ID NO:19 is the cas5 no NLS DNA sequence.

SEQ ID NO:20 is the cas6 no NLS DNA sequence.

SEQ ID NO:21 is the cas7 no NLS DNA sequence.

SEQ ID NO:22 is the cse1 no NLS DNA sequence.

SEQ ID NO:23 is the cse2 no NLS DNA sequence.

SEQ ID NO:24 is the cas5 N terminal NLS DNA sequence.

SEQ ID NO:25 is the cas5 C terminal NLS DNA sequence.

SEQ ID NO:26 is the cse1 N terminal NLS DNA sequence.

SEQ ID NO:27 is the cas6 C terminal NLS DNA sequence.

SEQ ID NO:28 is the cas7 C terminal NLS DNA sequence.

SEQ ID NO:29 is the cse2 C terminal NLS DNA sequence.

SEQ ID NO:30 is the cas5 N terminal NLS protein PRT sequence.

SEQ ID NO:31 is the cas5 C terminal NLS protein PRT sequence.

SEQ ID NO:32 is the cse1 N terminal NLS protein PRT sequence.

SEQ ID NO:33 is the cas6 C terminal NLS protein PRT sequence.

SEQ ID NO:34 is the cas7 C terminal NLS protein PRT sequence.

SEQ ID NO:35 is the cse2 C terminal NLS protein PRT sequence.

SEQ ID NO:36 is the Maize UBI promoter DNA sequence from Zea mays.

SEQ ID NO:37 is the Maize UBI 5 prime untranslated region DNA sequence from Zea mays.

SEQ ID NO:38 is the Maize UBI intron 1 DNA sequence from Zea mays.

SEQ ID NO:39 is the UBI cas5 no NLS DNA sequence.

SEQ ID NO:40 is the UBI cas6 no NLS DNA sequence.

SEQ ID NO:41 is the UBI cas7 no NLS DNA sequence.

SEQ ID NO:42 is the UBI cse1 no NLS DNA sequence.

SEQ ID NO:43 is the UBI cse2 no NLS DNA sequence.

SEQ ID NO:44 is the UBI cas5 N terminal NLS DNA sequence.

SEQ ID NO:45 is the UBI cas5 C terminal NLS DNA sequence.

SEQ ID NO:46 is the UBI cse1 N terminal NLS DNA sequence.

SEQ ID NO:47 is the UBI cas6 C terminal NLS DNA sequence.

SEQ ID NO:48 is the UBI cas7 C terminal NLS DNA sequence.

SEQ ID NO:49 is the UBI cse2 C terminal NLS DNA sequence.

SEQ ID NO:50 is the Transcriptional activator motif from AtCBF1 DNA sequence from Arabidopsis thaliana.

SEQ ID NO:51 is the cas5 gene with N-terminal NLS linked by 30XQ to AtCBF1 motif DNA sequence.

SEQ ID NO:52 is the cas5 gene with N-terminal NLS linked by GRA to AtCBF1 motif DNA sequence.

SEQ ID NO:53 is the cas5 gene with C-terminal NLS linked by 30XQ to AtCBF1 motif DNA sequence.

SEQ ID NO:54 is the cas5 gene with C-terminal NLS linked by GRA to AtCBF1 motif DNA sequence.

SEQ ID NO:55 is the cse1 gene with N-terminal NLS linked by 30XQ to AtCBF1 motif DNA sequence.

SEQ ID NO:56 is the cse1 gene with N-terminal NLS linked by GRA to AtCBF1 motif DNA sequence.

SEQ ID NO:57 is the protein of cas5 gene with N-terminal NLS linked by 30XQ to AtCBF1 motif PRT sequence.

SEQ ID NO:58 is the protein of cas5 gene with N-terminal NLS linked by GRA to AtCBF1 motif PRT sequence.

SEQ ID NO:59 is the protein of cas5 gene with C-terminal NLS linked by 30XQ to AtCBF1 motif PRT sequence.

SEQ ID NO:60 is the protein of cas5 gene with C-terminal NLS linked by GRA to AtCBF1 motif PRT sequence.

SEQ ID NO:61 is the protein of cse1 gene with N-terminal NLS linked by 30XQ to AtCBF1 motif PRT sequence.

SEQ ID NO:62 is the protein of cse1 gene with N-terminal NLS linked by GRA to AtCBF1 motif PRT sequence.

SEQ ID NO:63 is the Spy cas9 gene DNA sequence.

SEQ ID NO:64 is the protein from Spy dcas9 gene PRT sequence.

SEQ ID NO:65 is the Spy dcas9 gene linked with AtCBF1 transcriptional activator by 30XQ DNA sequence.

SEQ ID NO:66 is the Spy dcas9 gene linked with AtCBF1 transcriptional activator by GRA DNA sequence.

SEQ ID NO:67 is the protein of Spy dcas9 gene linked with AtCBF1 transcriptional activator by 30XQ PRT sequence.

SEQ ID NO:68 is the protein of Spy dcas9 gene linked with AtCBF1 transcriptional activator by GRA PRT sequence.

SEQ ID NO:69 is the UBI cas5 gene with N-terminal NLS linked by 30XQ to AtCBF1 motif DNA sequence.

SEQ ID NO:70 is the UBI cas5 gene with N-terminal NLS linked by GRA to AtCBF1 motif DNA sequence.

SEQ ID NO:71 is the UBI cas5 gene with C-terminal NLS linked by 30XQ to AtCBF1 motif DNA sequence.

SEQ ID NO:72 is the UBI cas5 gene with C-terminal NLS linked by GRA to AtCBF1 motif DNA sequence.

SEQ ID NO:73 is the UBI cse1 gene with N-terminal NLS linked by 30XQ to AtCBF1 motif DNA sequence.

SEQ ID NO:74 is the UBI cse1 gene with N-terminal NLS linked by GRA to AtCBF1 motif DNA sequence.

SEQ ID NO:75 is the UBI Spy dcas9 gene linked with AtCBF1 transcriptional activator by 30XQ DNA sequence.

SEQ ID NO:76 is the UBI Spy dcas9 gene linked with AtCBF1 transcriptional activator by GRA DNA sequence.

SEQ ID NO:77 is the Maize U6 polymerase III promoter DNA sequence from Zea mays.

SEQ ID NO:78 is the Mature Type I-E guide RNA RNA sequence.

SEQ ID NO:79 is the GACT targeting CRISPR array DNA sequence.

SEQ ID NO:80 is the Maize UBI polymerase II GACT guide RNA expression cassette DNA sequence.

SEQ ID NO:81 is the Maize U6 polymerase III GACT guide RNA expression cassette DNA sequence.

SEQ ID NO:82 is the Maize codon optimized cas3 gene DNA sequence.

SEQ ID NO:83 is the ST-LS1 intron 2 DNA sequence from Solanum tuberosum.

SEQ ID NO:84 is the Maize codon optimized cas3 gene with ST-LS1 intron 2 DNA sequence.

SEQ ID NO:85 is the Maize codon optimized cas3 nickase gene with ST-LS1 intron 2 DNA sequence.

SEQ ID NO:86 is the cas3 with N-terminal NLS DNA sequence.

SEQ ID NO:87 is the cas3 with C-terminal NLS DNA sequence.

SEQ ID NO:88 is the cas3 with both N- and C-terminal NLSs DNA sequence.

SEQ ID NO:89 is the cas3 nickase with N-terminal NLS DNA sequence.

SEQ ID NO:90 is the cas3 nickase with C-terminal NLS DNA sequence.

SEQ ID NO:91 is the cas3 nickase with both N- and C-terminal NLSs DNA sequence.

SEQ ID NO:92 is the protein of cas3 with N-terminal NLS PRT sequence.

SEQ ID NO:93 is the protein of cas3 with C-terminal NLS PRT sequence.

SEQ ID NO:94 is the protein of cas3 with both N- and C-terminal NLSs PRT sequence.

SEQ ID NO:95 is the protein of cas3 nickase with N-terminal NLS PRT sequence.

SEQ ID NO:96 is the protein of cas3 nickase with C-terminal NLS PRT sequence.

SEQ ID NO:97 is the protein of cas3 nickase with both N- and C-terminal NLSs PRT sequence.

SEQ ID NO:98 is the UBI cas3 with N-terminal NLS DNA sequence.

SEQ ID NO:99 is the UBI cas3 with C-terminal NLS DNA sequence.

SEQ ID NO:100 is the UBI cas3 with both N- and C-terminal NLSs DNA sequence.

SEQ ID NO:101 is the UBI cas3 nickase with N-terminal NLS DNA sequence.

SEQ ID NO:102 is the UBI cas3 nickase with C-terminal NLS DNA sequence.

SEQ ID NO:103 is the UBI cas3 nickase with both N- and C-terminal NLSs DNA sequence.

SEQ ID NO:104 is the Upstream binding region for Type I-E Cascade guide RNA complexes DNA sequence.

SEQ ID NO:105 is the Minimal 35S promoter from the cauliflower mosaic virus (CMV) DNA sequence from Cauliflower Mosaic Virus.

SEQ ID NO:106 is the 5 prime untranslated region (UTR) from the tobacco mosaic virus (TMV) DNA sequence from Tobacco Mosaic Virus.

SEQ ID NO:107 is the Gene encoding Ds-Red Express fluorescent protein DNA sequence.

SEQ ID NO:108 is the Rice Ubiquitin terminator DNA sequence from Oryza sativa.

SEQ ID NO:109 is the GACT expression cassette DNA sequence.

SEQ ID NO:110 is the cash gene with C-terminal linkage to AtCBF1 motif DNA sequence.

SEQ ID NO:111 is the cse1 gene with C-terminal linkage to AtCBF1 motif DNA sequence.

SEQ ID NO:112 is the UBI cas6 gene with C-terminal linkage to AtCBF1 motif DNA sequence.

SEQ ID NO:113 is the UBI cse1 gene with C-terminal linkage to AtCBF1 motif DNA sequence.

SEQ ID NO:114 is the GACT2 expression cassette DNA sequence.

SEQ ID NO:115 is the Spy dCas9 U6 polymerase III guide RNA expression construct targeting GACT2 DNA sequence.

SEQ ID NO:116 is the Sth7710 Cascade U6 polymerase III guide RNA expression construct targeting GACT2 DNA sequence.

SEQ ID NO:117 is the Sth7710 Cascade R1 protospacer target A2+ DNA sequence from Zea mays.

SEQ ID NO:118 is the Sth7710 Cascade R1 protospacer target A4+ DNA sequence from Zea mays.

SEQ ID NO:119 is the Sth7710 Cascade R1 protospacer target A1− DNA sequence from Zea mays.

SEQ ID NO:120 is the Spy dCas9 R1 protospacer target A3+ DNA sequence from Zea mays.

SEQ ID NO:121 is the Spy dCas9 R1 protospacer target A7+ DNA sequence from Zea mays.

SEQ ID NO:122 is the Spy dCas9 R1 protospacer target A2− DNA sequence from Zea mays.

SEQ ID NO:123 is the Sth7710 Cascade U6 polymerase III guide RNA expression construct targeting A2+ DNA sequence.

SEQ ID NO:124 is the Sth7710 Cascade U6 polymerase III guide RNA expression construct targeting A4+ DNA sequence.

SEQ ID NO:125 is the Sth7710 Cascade U6 polymerase III guide RNA expression construct targeting A1− DNA sequence.

SEQ ID NO:126 is the C1 coding DNA sequence DNA sequence from Zea mays.

SEQ ID NO:127 is the Cauliflower Mosaic Virus 35S enhancer DNA sequence from Cauliflower Mosaic Virus.

SEQ ID NO:128 is the Cauliflower Mosaic Virus promoter DNA sequence from Cauliflower Mosaic Virus.

SEQ ID NO:129 is the Alcohol dehydrogenase 1 intron 1 DNA sequence from Zea mays.

SEQ ID NO:130 is the Cl gene over-expression cassette DNA sequence.

SEQ ID NO:131 is the R1 coding DNA sequence DNA sequence from Zea mays.

SEQ ID NO:132 is the R1 gene over-expression cassette DNA sequence.

SEQ ID NO:133 is the Spy dCas9 U6 polymerase III guide RNA expression construct targeting A3+ DNA sequence.

SEQ ID NO:134 is the Spy dCas9 U6 polymerase III guide RNA expression construct targeting A7+ DNA sequence.

SEQ ID NO:135 is the Spy dCas9 U6 polymerase III guide RNA expression construct targeting A2− DNA sequence.

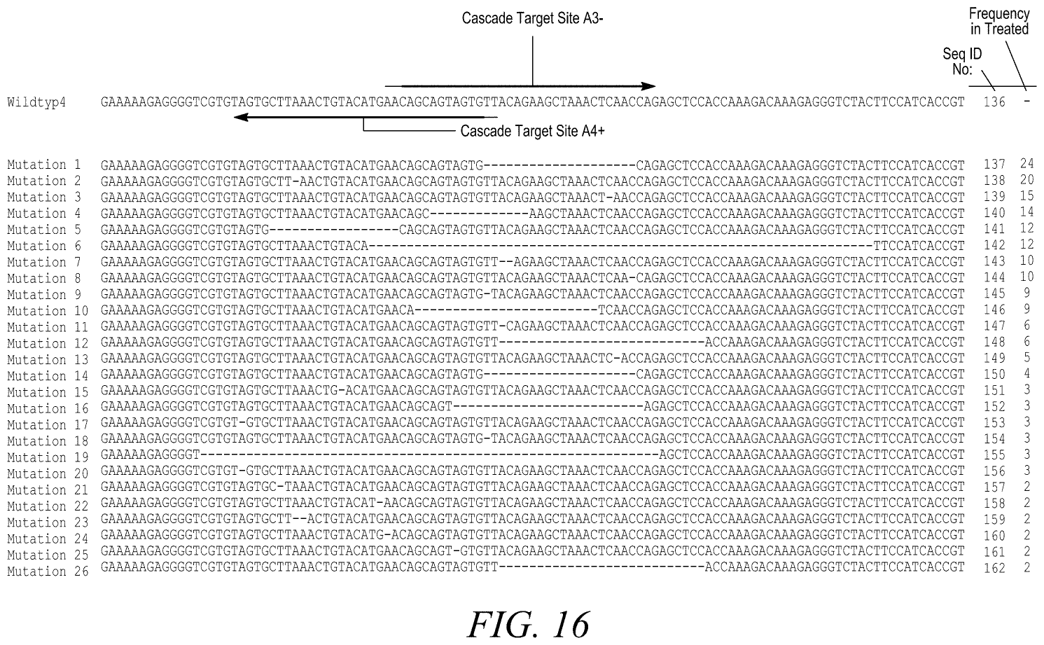

SEQ ID NO:136 is the Wildtype targeting sequence-FIG.16 DNA sequence from Zea mays.

SEQ ID NO:137 is the Mutation 1-FIG.16 DNA sequence.

SEQ ID NO:138 is the Mutation 2-FIG.16 DNA sequence.

SEQ ID NO:139 is the Mutation 3-FIG.16 DNA sequence.

SEQ ID NO:140 is the Mutation 4-FIG.16 DNA sequence.

SEQ ID NO:141 is the Mutation 5-FIG.16 DNA sequence.

SEQ ID NO:142 is the Mutation 6-FIG.16 DNA sequence.

SEQ ID NO:143 is the Mutation 7-FIG.16 DNA sequence.

SEQ ID NO:144 is the Mutation 8-FIG.16 DNA sequence.

SEQ ID NO:145 is the Mutation 9-FIG.16 DNA sequence.

SEQ ID NO:146 is the Mutation 10-FIG.16 DNA sequence.

SEQ ID NO:147 is the Mutation 11-FIG.16 DNA sequence.

SEQ ID NO:148 is the Mutation 12-FIG.16 DNA sequence.

SEQ ID NO:149 is the Mutation 13-FIG.16 DNA sequence.

SEQ ID NO:150 is the Mutation 14-FIG.16 DNA sequence.

SEQ ID NO:151 is the Mutation 15-FIG.16 DNA sequence.

SEQ ID NO:152 is the Mutation 16-FIG.16 DNA sequence.

SEQ ID NO:153 is the Mutation 17-FIG.16 DNA sequence.

SEQ ID NO:154 is the Mutation 18-FIG.16 DNA sequence.

SEQ ID NO:155 is the Mutation 19-FIG.16 DNA sequence.

SEQ ID NO:156 is the Mutation 20-FIG.16 DNA sequence.

SEQ ID NO:157 is the Mutation 21-FIG.16 DNA sequence.

SEQ ID NO:158 is the Mutation 22-FIG.16 DNA sequence.

SEQ ID NO:159 is the Mutation 23-FIG.16 DNA sequence.

SEQ ID NO:160 is the Mutation 24-FIG.16 DNA sequence.

SEQ ID NO:161 is the Mutation 25-FIG.16 DNA sequence.

SEQ ID NO:162 is the Mutation 26-FIG.16 DNA sequence.

SEQ ID NO:163 is the Sth7710 Cascade R1 protospacer target A3− DNA sequence from Zea mays.

SEQ ID NO:164 is the Sth7710 Cascade U6 polymerase III guide RNA expression construct targeting A3− DNA sequence.

SEQ ID NO:165 is the Sequence encoding I-TevI amino terminal nuclease domain DNA sequence from Escherichia virus T4.

SEQ ID NO:166 is the sequence encoding I-TevI amino terminal nuclease domain DNA sequence.

SEQ ID NO:167 is the nuclease enabled I-TevI:cas6:NLS DNA sequence.

SEQ ID NO:168 is the nuclease enabled I-TevI:GRA:cas6:NLS DNA sequence.

SEQ ID NO:169 is the nuclease enabled I-TevI:(G2S)3:cas6:NLS DNA sequence.

SEQ ID NO:170 is the nuclease enabled I-TevI:XTEN:cas6:NLS DNA sequence.

SEQ ID NO:171 is the nuclease enabled I-TevI:cse1:NLS DNA sequence.

SEQ ID NO:172 is the nuclease enabled I-TevI:GRA:cse1:NLS DNA sequence.

SEQ ID NO:173 is the nuclease enabled I-TevI:(G2S)3:cse1:NLS DNA sequence.

SEQ ID NO:174 is the nuclease enabled I-TevI:XTEN:csel:NLS DNA sequence.

SEQ ID NO:175 is the Protein of nuclease enabled I-TevI:cas6:NLS PRT sequence.

SEQ ID NO:176 is the Protein of nuclease enabled I-TevI:GRA:cas6:NLS PRT sequence.

SEQ ID NO:177 is the Protein of nuclease enabled I-TevI:(G2S)3:cas6:NLS PRT sequence.

SEQ ID NO:178 is the Protein of nuclease enabled I-TevI:XTEN:cas6:NLS PRT sequence.

SEQ ID NO:179 is the Protein of nuclease enabled I-TevI:cse1:NLS PRT sequence.

SEQ ID NO:180 is the Protein of nuclease enabled I-TevI:GRA:cse1:NLS PRT sequence.

SEQ ID NO:181 is the Protein of nuclease enabled I-TevI:(G2S)3:cse1:NLS PRT sequence.

SEQ ID NO:182 is the Protein of nuclease enabled I-TevI:XTEN:cse1:NLS PRT sequence.

SEQ ID NO:183 is the UBI nuclease enabled I-TevI:cas6:NLS DNA sequence.

SEQ ID NO:184 is the UBI nuclease enabled I-TevI:GRA:cas6:NLS DNA sequence.

SEQ ID NO:185 is the UBI nuclease enabled I-TevI:(G2S)3:cas6:NLS DNA sequence.

SEQ ID NO:186 is the UBI nuclease enabled I-TevI:XTEN:cas6:NLS DNA sequence.

SEQ ID NO:187 is the UBI nuclease enabled I-TevI:cse1:NLS DNA sequence.

SEQ ID NO:188 is the UBI nuclease enabled I-TevI:GRA:cse1:NLS DNA sequence.

SEQ ID NO:189 is the UBI nuclease enabled I-TevI:(G2S)3:cse1:NLS DNA sequence.

SEQ ID NO:190 is the UBI nuclease enabled I-TevI:XTEN:csel:NLS DNA sequence.

SEQ ID NO:191 is the Immunofluorescent cascade target sequence DNA sequence.

SEQ ID NO:192 is the Maize Histone 2B promoter DNA sequence from Zea mays.

SEQ ID NO:193 is the Potato Proteinase Inhibitor II terminator DNA sequence from Solanum tuberosum.

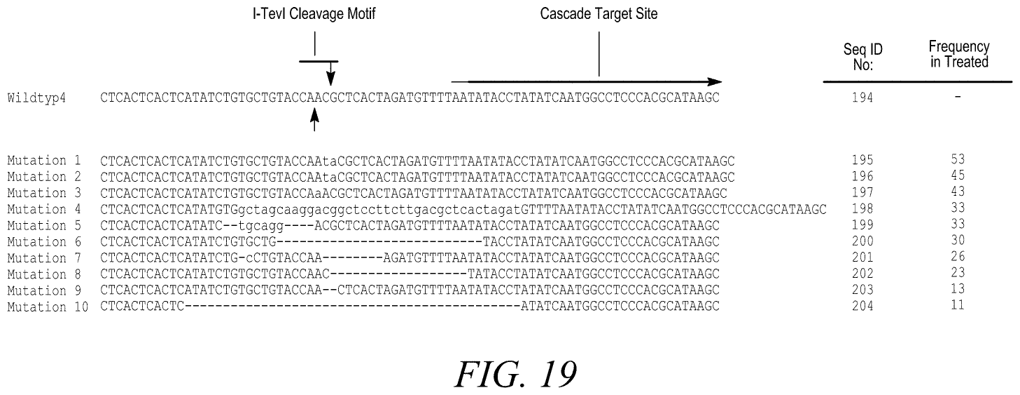

SEQ ID NO:194 is the Wildtype targeting sequence-FIG. 19 DNA sequence.

SEQ ID NO:195 is the Mutation 1-FIG. 19 DNA sequence.

SEQ ID NO:196 is the Mutation 2-FIG. 19 DNA sequence.

SEQ ID NO:197 is the Mutation 3-FIG. 19 DNA sequence.

SEQ ID NO:198 is the Mutation 4-FIG. 19 DNA sequence.

SEQ ID NO:199 is the Mutation 5-FIG. 19 DNA sequence.

SEQ ID NO:200 is the Mutation 6-FIG. 19 DNA sequence.

SEQ ID NO:201 is the Mutation 7-FIG. 19 DNA sequence.

SEQ ID NO:202 is the Mutation 8-FIG.19 DNA sequence.

SEQ ID NO:203 is the Mutation 9-FIG. 19 DNA sequence.

SEQ ID NO:204 is the Mutation 10-FIG. 19 DNA sequence.

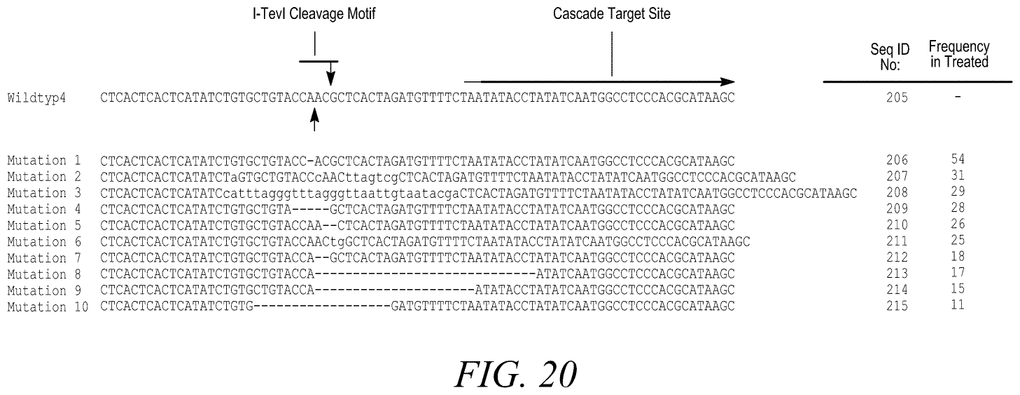

SEQ ID NO:205 is the Wildtype targeting sequence-FIG. 20 DNA sequence.

SEQ ID NO:206 is the Mutation 1-FIG. 20 DNA sequence.

SEQ ID NO:207 is the Mutation 2-FIG. 20 DNA sequence.

SEQ ID NO:208 is the Mutation 3-FIG. 20 DNA sequence.

SEQ ID NO:209 is the Mutation 4-FIG. 20 DNA sequence.

SEQ ID NO:210 is the Mutation 5-FIG. 20 DNA sequence.

SEQ ID NO:211 is the Mutation 6-FIG. 20 DNA sequence.

SEQ ID NO:212 is the Mutation 7-FIG. 20 DNA sequence.

SEQ ID NO:213 is the Mutation 8-FIG. 20 DNA sequence.

SEQ ID NO:214 is the Mutation 9-FIG. 20 DNA sequence.

SEQ ID NO:215 is the Mutation 10-FIG. 20 DNA sequence.

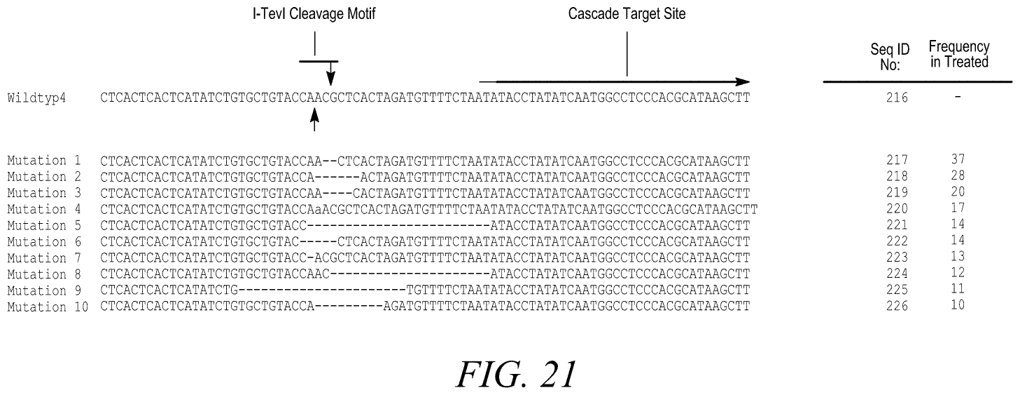

SEQ ID NO:216 is the Wildtype targeting sequence-FIG. 21 DNA sequence.

SEQ ID NO:217 is the Mutation 1-FIG. 21 DNA sequence.

SEQ ID NO:218 is the Mutation 2-FIG. 21 DNA sequence.

SEQ ID NO:219 is the Mutation 3-FIG. 21 DNA sequence.

SEQ ID NO:220 is the Mutation 4-FIG. 21 DNA sequence.

SEQ ID NO:221 is the Mutation 5-FIG. 21 DNA sequence.

SEQ ID NO:222 is the Mutation 6-FIG. 21 DNA sequence.

SEQ ID NO:223 is the Mutation 7-FIG. 21 DNA sequence.

SEQ ID NO:224 is the Mutation 8-FIG. 21 DNA sequence.

SEQ ID NO:225 is the Mutation 9-FIG. 21 DNA sequence.

SEQ ID NO:226 is the Mutation 10-FIG. 21 DNA sequence.

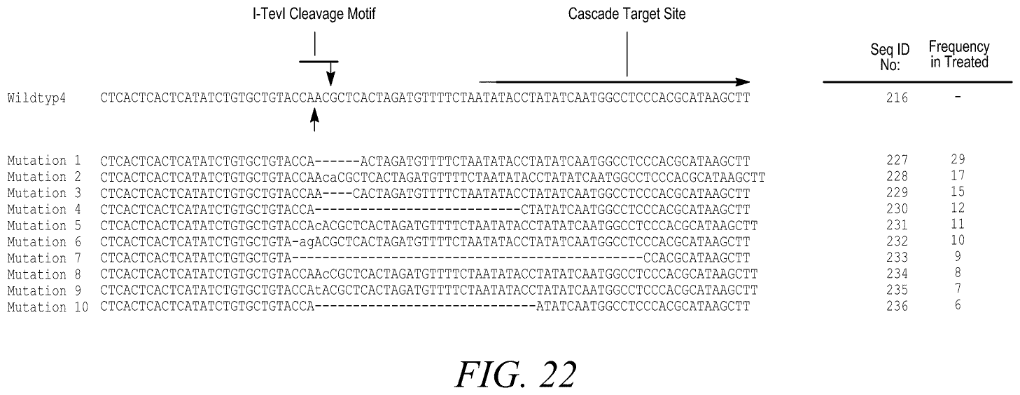

SEQ ID NO:227 is the Mutation 1-FIG. 22 DNA sequence.

SEQ ID NO:228 is the Mutation 2-FIG. 22 DNA sequence.

SEQ ID NO:229 is the Mutation 3-FIG. 22 DNA sequence.

SEQ ID NO:230 is the Mutation 4-FIG. 22 DNA sequence.

SEQ ID NO:231 is the Mutation 5-FIG. 22 DNA sequence.

SEQ ID NO:232 is the Mutation 6-FIG. 22 DNA sequence.

SEQ ID NO:233 is the Mutation 7-FIG. 22 DNA sequence.

SEQ ID NO:234 is the Mutation 8-FIG. 22 DNA sequence.

SEQ ID NO:235 is the Mutation 9-FIG. 22 DNA sequence.

SEQ ID NO:236 is the Mutation 10-FIG. 22 DNA sequence.

SEQ ID NO: 237 is the Streptococcus mutans cas5 gene sequence.

SEQ ID NO: 238 is the Streptococcus mutans cas6 gene sequence.

SEQ ID NO: 239 is the Streptococcus mutans cas7 gene sequence.

SEQ ID NO: 240 is the Streptococcus mutans cse1 gene sequence.

SEQ ID NO: 241 is the Streptococcus mutans cse2 gene sequence.

SEQ ID NO: 242 is the Streptococcus mutans cas3 gene sequence.

SEQ ID NO: 243 is the Peptostreptococcus anaerobius cas5 gene sequence.

SEQ ID NO: 244 is the Peptostreptococcus anaerobius cas6 gene sequence.

SEQ ID NO: 245 is the Peptostreptococcus anaerobius cas7 gene sequence.

SEQ ID NO: 246 is the Peptostreptococcus anaerobius cse1 gene sequence.

SEQ ID NO: 247 is the Peptostreptococcus anaerobius cse2 gene sequence.

SEQ ID NO: 248 is the Peptostreptococcus anaerobius cas3 gene sequence.

SEQ ID NO: 249 is the Gardnerella vaginalis cas5 gene sequence.

SEQ ID NO: 250 is the Gardnerella vaginalis cas6 gene sequence.

SEQ ID NO: 251 is the Gardnerella vaginalis cas7 gene sequence.

SEQ ID NO: 252 is the Gardnerella vaginalis cse1 gene sequence.

SEQ ID NO: 253 is the Gardnerella vaginalis cse2 gene sequence.

SEQ ID NO: 254 is the Gardnerella vaginalis cas3 gene sequence.

SEQ ID NO: 255 is the Pseudomonas sp. S-6-2 cas5 gene sequence.

SEQ ID NO: 256 is the Pseudomonas sp. S-6-2 cas6 gene sequence.

SEQ ID NO: 257 is the Pseudomonas sp. S-6-2 cas7 gene sequence.

SEQ ID NO: 258 is the Pseudomonas sp. S-6-2 cse1 gene sequence.

SEQ ID NO: 259 is the Pseudomonas sp. S-6-2 cse2 gene sequence.

SEQ ID NO: 260 is the Pseudomonas sp. S-6-2 cas3 gene sequence.

SEQ ID NO: 261 is the Ralstonia solanacearum cas5 gene sequence.

SEQ ID NO: 262 is the Ralstonia solanacearum cas6 gene sequence.

SEQ ID NO: 263 is the Ralstonia solanacearum cas7 gene sequence.

SEQ ID NO: 264 is the Ralstonia solanacearum cse1 gene sequence.

SEQ ID NO: 265 is the Ralstonia solanacearum cse2 gene sequence.

SEQ ID NO: 266 is the Ralstonia solanacearum cas3 gene sequence.

SEQ ID NO: 267 is the Streptococcus mutans Cas5 protein sequence.

SEQ ID NO: 268 is the Streptococcus mutans Cas6 protein sequence.

SEQ ID NO: 269 is the Streptococcus mutans Cas7 protein sequence.

SEQ ID NO: 270 is the Streptococcus mutans Cse1 protein sequence.

SEQ ID NO: 271 is the Streptococcus mutans Cse2 protein sequence.

SEQ ID NO: 272 is the Streptococcus mutans Cas3 protein sequence.

SEQ ID NO: 273 is the Peptostreptococcus anaerobius Cas5 protein sequence.

SEQ ID NO: 274 is the Peptostreptococcus anaerobius Cas6 protein sequence.

SEQ ID NO: 275 is the Peptostreptococcus anaerobius Cas7 protein sequence.

SEQ ID NO: 276 is the Peptostreptococcus anaerobius Cse1 protein sequence.

SEQ ID NO: 277 is the Peptostreptococcus anaerobius Cse2 protein sequence.

SEQ ID NO: 278 is the Peptostreptococcus anaerobius Cas3 protein sequence.

SEQ ID NO: 279 is the Gardnerella vaginalis Cas5 protein sequence.

SEQ ID NO: 280 is the Gardnerella vaginalis Cas6 protein sequence.

SEQ ID NO: 281 is the Gardnerella vaginalis Cas7 protein sequence.

SEQ ID NO: 282 is the Gardnerella vaginalis Cse1 protein sequence.

SEQ ID NO: 283 is the Gardnerella vaginalis Cse2 protein sequence.

SEQ ID NO: 284 is the Gardnerella vaginalis Cas3 protein sequence.

SEQ ID NO: 285 is the Pseudomonas sp. S-6-2 Cas5 protein sequence.

SEQ ID NO: 286 is the Pseudomonas sp. S-6-2 Cas6 protein sequence.

SEQ ID NO: 287 is the Pseudomonas sp. S-6-2 Cas7 protein sequence.

SEQ ID NO: 288 is the Pseudomonas sp. S-6-2 Cse1 protein sequence.

SEQ ID NO: 289 is the Pseudomonas sp. S-6-2 Cse2 protein sequence.

SEQ ID NO: 290 is the Pseudomonas sp. S-6-2 Cas3 protein sequence.

SEQ ID NO: 291 is the Ralstonia solanacearum Cas5 protein sequence.

SEQ ID NO: 292 is the Ralstonia solanacearum Cas6 protein sequence.

SEQ ID NO: 293 is the Ralstonia solanacearum Cas7 protein sequence.

SEQ ID NO: 294 is the Ralstonia solanacearum Cse1 protein sequence.

SEQ ID NO: 295 is the Ralstonia solanacearum Cse2 protein sequence.

SEQ ID NO: 296 is the Ralstonia solanacearum Cas3 protein sequence.

### DETAILED DESCRIPTION

Compositions and methods are provided for novel CRISPR effector systems and elements comprising such systems, including, but not limiting to, novel guide polynucleotide/endonuclease complexes, guide polynucleotides, guide RNA elements, Cas proteins, and endonucleases, as well as proteins comprising an endonuclease functionality (domain) as well as at least one other functionality (domain) such as, but not limited to, a protein subunit capable of forming a cascade. Compositions and methods are also provided for direct delivery of endonucleases, cleavage ready cascades, guide RNAs and guide RNA/endonuclease complexes. The present disclosure further includes compositions and methods for genome modification of a target sequence in the genome of a cell, for gene editing, and for inserting a polynucleotide of interest into the genome of a cell.

Terms used in the claims and specification are defined as set forth below unless otherwise specified. It must be noted that, as used in the specification and the appended claims, the singular forms “a,” “an” and “the” include plural referents unless the context clearly dictates otherwise.

### Definitions

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally comprising synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

“Open reading frame” is abbreviated ORF.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “substantially complementary” refers to the relationship between a first polynucleotide sequence and a second polynucleotide sequence. In some aspects, a first polynucleotide sequence shares at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with a second polynucleotide sequence over the length of either the first or second polynucleotide, or a fragment of either. In some aspects, a first polynucleotide sequence differs from a second polynucleotide sequence by one nucleotide, by two, three, four, five, six, seven, eight, nine, ten, 11, 12, 13, 14, 15, 16, 17, 18, 19, 20, or greater than 20 nucleotides, over the length of either the first or the second polynucleotide, or a fragment of either. In some aspects, the first polynucleotide sequence is the same length as the second polynucleotide sequence. In some aspects, the first polynucleotide sequence is longer than the second polynucleotide sequence. In some aspects, the first polynucleotide sequence is shorter than the second polynucleotide sequence.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length. Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2× SSC (20× SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1× SSC at 55 to 60° C. Exemplary high stringency conditions include hybridization in 50% formamide, 1 M NaCl, 1% SDS at 37° C., and a wash in 0.1× SSC at 60 to 65° C.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events: the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) Mol Cell Biol 4:2253-8; Ayares et al., (1986) Proc. Natl. Acad. Sci. USA 83:5199-203; Liskay et al., (1987) Genetics 115:161-7.

“Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, Wis.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al., (1992) Comput Appl Biosci 8:189-191) and found in the MegAlign™ program of the LASERGENE bioinformatics computing suite (DNASTAR Inc., Madison, Wis.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program. The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign™ v6.1 program of the LASERGENE bioinformatics computing suite (DNASTAR Inc., Madison, WI). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program. Unless otherwise stated, sequence identity/similarity values provided herein refer to the value obtained using GAP Version 10 (GCG, Accelrys, San Diego, Calif.) using the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89:10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases. “BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence. It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any percentage from 50% to 100%. Indeed, any amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Polynucleotide and polypeptide sequences, variants thereof, and the structural relationships of these sequences can be described by the terms “homology”, “homologous”, “substantially identical”, “substantially similar” and “corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment. Substantially similar nucleic acid sequences encompassed may be defined by their ability to hybridize (under moderately stringent conditions, e.g., 0.5× SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

An “isolated” or “purified” nucleic acid molecule, polynucleotide, polypeptide, or protein, or biologically active portion thereof, is substantially or essentially free from components that normally accompany or interact with the polynucleotide or protein as found in its naturally occurring environment. Thus, an isolated or purified polynucleotide or polypeptide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can comprise less than about 5 kb, four kb, 3 kb, 2 kb, 1 kb, 0.5 kb, or 0.1 kb of nucleotide sequence that naturally flank the polynucleotide in genomic DNA of the cell from which the polynucleotide is derived. Isolated polynucleotides may be purified from a cell in which they naturally occur. Conventional nucleic acid purification methods known to skilled artisans may be used to obtain isolated polynucleotides. The term also embraces recombinant polynucleotides and chemically synthesized polynucleotides.

The term “fragment” refers to a contiguous set of nucleotides or amino acids. In one embodiment, a fragment is 2, 3, 4, 5, 6, 7 8, 9, 10, 11, 12, 13, 14, 15, 16, 17, 18, 19, 20, or greater than 20 contiguous nucleotides. In one embodiment, a fragment is 2, 3, 4, 5, 6, 7 8, 9, 10, 11, 12, 13, 14, 15, 16, 17, 18, 19, 20, or greater than 20 contiguous amino acids. A fragment may or may not exhibit the function of a sequence sharing some percent identity over the length of said fragment.

The terms “fragment that is functionally equivalent” and “functionally equivalent fragment” are used interchangeably herein. These terms refer to a portion or subsequence of an isolated nucleic acid fragment or polypeptide that displays the same activity or function as the longer sequence from which it derives. In one example, the fragment retains the ability to alter gene expression or produce a certain phenotype whether or not the fragment encodes an active protein. For example, the fragment can be used in the design of genes to produce the desired phenotype in a modified plant. Genes can be designed for use in suppression by linking a nucleic acid fragment, whether or not it encodes an active enzyme, in the sense or antisense orientation relative to a plant promoter sequence.

“Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in its natural endogenous location with its own regulatory sequences.

By the term “endogenous” it is meant a sequence or other molecule that naturally occurs in a cell or organism. In one aspect, an endogenous polynucleotide is normally found in the genome of a cell; that is, not heterologous.

An “allele” is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

As used herein, a “targeted mutation” is a mutation in a gene (referred to as the target gene), including a native gene, that was made by altering a target sequence within the target gene using any method known to one skilled in the art, including a method involving a guided Cas endonuclease system as disclosed herein.

The terms “knock-out”, “gene knock-out” and “genetic knock-out” are used interchangeably herein. A knock-out represents a DNA sequence of a cell that has been rendered partially or completely inoperative by targeting with a Cas protein; for example, a DNA sequence prior to knock-out could have encoded an amino acid sequence, or could have had a regulatory function (e.g., promoter).

The terms “knock-in”, “gene knock-in”, “gene insertion” and “genetic knock-in” are used interchangeably herein. A knock-in represents the replacement or insertion of a DNA sequence at a specific DNA sequence in cell by targeting with a Cas protein (for example by homologous recombination (HR), wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

By “domain” it is meant a contiguous stretch of nucleotides (that can be RNA, DNA, and/or RNA-DNA-combination sequence) or amino acids.

The term “conserved domain” or “motif” means a set of polynucleotides or amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

A “codon-modified gene” or “codon-preferred gene” or “codon-optimized gene” is a gene having its frequency of codon usage designed to mimic the frequency of preferred codon usage of the host cell.

An “optimized” polynucleotide is a sequence that has been optimized for improved expression in a particular heterologous host cell.

A “plant-optimized nucleotide sequence” is a nucleotide sequence that has been optimized for expression in plants, particularly for increased expression in plants. A plant-optimized nucleotide sequence includes a codon-optimized gene. A plant-optimized nucleotide sequence can be synthesized by modifying a nucleotide sequence encoding a protein such as, for example, a Cas endonuclease as disclosed herein, using one or more plant-preferred codons for improved expression. See, for example, Campbell and Gowri (1990) Plant Physiol. 92:1-11 for a discussion of host-preferred codon usage.

A “promoter” is a region of DNA involved in recognition and binding of RNA polymerase and other proteins to initiate transcription. The promoter sequence comprises proximal and more distal upstream elements, the latter elements often referred to as enhancers. An “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. A RNA transcript is referred to as the mature RNA or mRNA when it is a RNA sequence derived from post-transcriptional processing of the primary transcript pre-mRNA. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “genome” refers to the entire complement of genetic material (genes and non-coding sequences) that is present in each cell of an organism, or virus or organelle; and/or a complete set of chromosomes inherited as a (haploid) unit from one parent.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In some embodiments, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, an insect cell, a mammalian cell, a pig cell, a cow cell, a goat cell, a sheep cell, a rodent cell, a rat cell, a mouse cell, a non-human primate cell, and a human cell. In some cases, the cell is in vitro. In some cases, the cell is in vivo.

The term “recombinant” refers to an artificial combination of two otherwise separated segments of sequence, e.g., by chemical synthesis, or manipulation of isolated segments of nucleic acids by genetic engineering techniques.

The terms “plasmid”, “vector” and “cassette” refer to a linear or circular extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector comprising a gene and having elements in addition to the gene that facilitates transformation of a particular host cell. “Expression cassette” refers to a specific vector comprising a gene and having elements in addition to the gene that allow for expression of that gene in a host.

The terms “recombinant DNA molecule”, “recombinant DNA construct”, “expression construct”, “construct”, and “recombinant construct” are used interchangeably herein. A recombinant DNA construct comprises an artificial combination of nucleic acid sequences, e.g., regulatory and coding sequences that are not all found together in nature. For example, a recombinant DNA construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to introduce the vector into the host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J4:2411-2418; De Almeida et al., (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The term “heterologous” refers to the difference between the original environment, location, or composition of a particular polynucleotide or polypeptide sequence and its current environment, location, or composition. Non-limiting examples include differences in taxonomic derivation (e.g., a polynucleotide sequence obtained from Zea mays would be heterologous if inserted into the genome of an Oryza sativa plant, or of a different variety or cultivar of Zea mays; or a polynucleotide obtained from a bacterium was introduced into a cell of a plant), or sequence (e.g., a polynucleotide sequence obtained from Zea mays, isolated, modified, and re-introduced into a maize plant). As used herein, “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more regulatory region(s) and/or a polynucleotide provided herein may be entirely synthetic.

The term “expression”, as used herein, refers to the production of a functional end-product (e.g., an mRNA, guide RNA, or a protein) in either precursor or mature form.

A “mature” protein refers to a post-translationally processed polypeptide (i.e., one from which any pre- or propeptides present in the primary translation product have been removed).

“Precursor” protein refers to the primary product of translation of mRNA (i.e., with pre- and propeptides still present). Pre- and propeptides may be but are not limited to intracellular localization signals.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; WO2007025097, published 1 Mar. 2007). A CRISPR locus can comprise of a CRISPR array, comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes but is not limited to: Cas9, Cpf1 (Cas12), C2c1, C2c2, C2c3, Cas3, Cas3-HD, Cas 5, Cas6, Cas7, Cas8, Cas8a, Cas8b, Cas8c, Cas8d, Cas8e, Cas8f, Cas10, Cse1, Cse2, or combinations or complexes or variants of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence. A Cas endonuclease described herein comprises one or more nuclease domains. A Cas protein, which may in some cases be a Cas effector protein or Cas endonuclease, is further defined as a functional fragment or functional variant of a native Cas protein, or a protein that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity.

A “functional fragment ”, “fragment that is functionally equivalent” and “functionally equivalent fragment” of a Cas endonuclease are used interchangeably herein, and refer to a portion or subsequence of the Cas endonuclease of the present disclosure in which the ability to recognize, bind to, and optionally unwind, nick or cleave (introduce a single or double-strand break in) the target site is retained. The portion or subsequence of the Cas endonuclease can comprise a complete or partial (functional) peptide of any of its domains such as for example, but not limiting to a complete of functional part of a Cas3 HD domain, a complete of functional part of a Cas3 Helicase domain, or a complete of functional part of a cascade protein (such as but not limiting to a Cas5, Cas7, and Cas8, or any variation of those components).

The terms “functional variant”, “variant that is functionally equivalent” and “functionally equivalent variant” of a Cas protein or endonuclease, including Cas proteins described herein, are used interchangeably herein, and refer to a variant of the Cas protein in which the ability to perform its native function (e.g., recognize, bind, unwind, nick, or cleave all or part of a target sequence) is retained.

A Cas endonuclease may also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a cascade (comprises at least a second protein domain that can form a cascade with other proteins). In one aspect, the multifunctional Cas endonuclease comprises at least one additional protein domain relative (either internally, upstream (5′), downstream (3′), or both internally 5′ and 3′, or any combination thereof) to those domains typical of a Cas endonuclease.

The terms “cascade”, “Cascade”, and “cascade complex” are used interchangeably herein. cascade (CRISPR-associated complex for antiviral defense) comprises a plurality of proteins that interact to form a complex. Cascade can assemble with a polynucleotide, to form a polynucleotide-protein complex (PNP). Cascade relies on the polynucleotide for complex assembly and stability, and for the identification of target nucleic acid sequences. Cascade functions as a surveillance complex that finds and optionally binds target nucleic acids that are complementary to a variable targeting domain of the guide polynucleotide.

The terms “cleavage-ready cascade”, “crCascade”, “cleavage-ready cascade complex”, “crCascade complex”, “cleavage-ready cascade system”, “CRC” and “crCascade system”, are used interchangeably herein and include reference to a multi-subunit protein complex that can assemble with a polynucleotide forming a polynucleotide-protein complex (PNP), wherein one of the cascade proteins is a Cas endonuclease capable of recognizing, binding to, and optionally unwinding, nicking, or cleaving all or part of a target sequence.

The terms “5′-cap” and “7-methylguanylate (m7G) cap” are used interchangeably herein. A 7-methylguanylate residue is located on the 5′ terminus of messenger RNA (mRNA) in eukaryotes. RNA polymerase II (PolII) transcribes mRNA in eukaryotes. Messenger RNA capping occurs generally as follows: The most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5′-5′ triphosphate-linked guanine at the transcript terminus. Finally, the 7-nitrogen of this terminal guanine is methylated by a methyl transferase.

The terminology “not having a 5′-cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′-capped RNA is subject to nuclear export. One or more RNA components herein are uncapped.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be a RNA sequence, a DNA sequence, or a combination thereof (a RNA-DNA combination sequence).

The terms “functional fragment”, “fragment that is functionally equivalent” and “functionally equivalent fragment” of a guide RNA, crRNA or tracrRNA are used interchangeably herein, and refer to a portion or subsequence of the guide RNA, crRNA or tracrRNA, respectively, of the present disclosure in which the ability to function as a guide RNA, crRNA or tracrRNA, respectively, is retained.

The terms “functional variant ”, “variant that is functionally equivalent” and “functionally equivalent variant” of a guide RNA, crRNA or tracrRNA (respectively) are used interchangeably herein, and refer to a variant of the guide RNA, crRNA or tracrRNA, respectively, of the present disclosure in which the ability to function as a guide RNA, crRNA or tracrRNA, respectively, is retained.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example US20150059010A1, published 26 Feb. 2015), or any combination thereof.

As used herein, the terms “guide polynucleotide/Cas endonuclease complex”, “guide polynucleotide/Cas endonuclease system”, “ guide polynucleotide/Cas complex”, “guide polynucleotide/Cas system” and “guided Cas system” “Polynucleotide-guided endonuclease”, “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327:167-170; Makarova et al. 2015, Nature Reviews Microbiology Vol. 13:1-15; Zetsche et al., 2015, Cell 163, 1-13; Shmakov et al., 2015, Molecular Cell 60, 1-13).

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex , wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

The terms “target site”, “target sequence”, “target site sequence”, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave . The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

An “altered target site”, “altered target sequence”, “modified target site”, “modified target sequence” are used interchangeably herein and refer to a target sequence as disclosed herein that comprises at least one alteration when compared to non-altered target sequence. Such “modifications” include, for example: replacement of at least one nucleotide, a deletion of at least one nucleotide, an insertion of at least one nucleotide, a substitution of at least one nucleotide, a chemical modification or chemical alteration of at least one nucleotide (e.g., the addition, deletion, or substitution of at least one atom), or any combination of the preceding. The terms “altered” and “modified” and their derivatives are used interchangeably.

A “modified nucleotide” or “edited nucleotide” refers to a nucleotide sequence of interest that comprises at least one alteration when compared to its non-modified nucleotide sequence. Such “modifications” include, for example: replacement of at least one nucleotide, a deletion of at least one nucleotide, an insertion of at least one nucleotide, a substitution of at least one nucleotide, a chemical modification or chemical alteration of at least one nucleotide (e.g., the addition, deletion, or substitution of at least one atom), or any combination of the preceding.

Methods for “modifying a target site” and “altering a target site” are used interchangeably herein and refer to methods for producing an altered target site.

As used herein, “donor DNA” is a DNA construct that comprises a polynucleotide of interest to be inserted into the target site of a Cas endonuclease.

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The term “plant-optimized Cas endonuclease” herein refers to a Cas protein, including a multifunctional Cas protein, encoded by a nucleotide sequence that has been optimized for expression in a plant cell or plant.

A “plant-optimized nucleotide sequence encoding a Cas endonuclease”, “plant-optimized construct encoding a Cas endonuclease” and a “plant-optimized polynucleotide encoding a Cas endonuclease” are used interchangeably herein and refer to a nucleotide sequence encoding a Cas protein, or a variant or functional fragment thereof, that has been optimized for expression in a plant cell or plant. A plant comprising a plant-optimized Cas endonuclease includes a plant comprising the nucleotide sequence encoding for the Cas sequence and/or a plant comprising the Cas endonuclease protein. In one aspect, the plant-optimized Cas endonuclease nucleotide sequence is a , rice-optimized, wheat-optimized, soybean-optimized, cotton-optimized, or canola-optimized Cas endonuclease.

The term “plant” generically includes whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. A “plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). The term “plant organ” refers to plant tissue or a group of tissues that constitute a morphologically and functionally distinct part of a plant. As used herein, a “plant element” is synonymous to a “portion” of a plant, and refers to any part of the plant, and can include distinct tissues and/or organs, and may be used interchangeably with the term “tissue” throughout. Similarly, a “plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keiki, or bud. The plant element may be in plant or in a plant organ, tissue culture, or cell culture.

“Progeny” comprises any subsequent generation of a plant.

As used herein, the term “plant part” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The term “monocotyledonous” or “monocot” refers to the subclass of angiosperm plants also known as “monocotyledoneae”, whose seeds typically comprise only one embryonic leaf, or cotyledon. The term includes references to whole plants, plant elements, plant organs (e.g., leaves, stems, roots, etc.), seeds, plant cells, and progeny of the same.

The term “dicotyledonous” or “dicot” refers to the subclass of angiosperm plants also knows as “dicotyledoneae”, whose seeds typically comprise two embryonic leaves, or cotyledons. The term includes references to whole plants, plant elements, plant organs (e.g., leaves, stems, roots, etc.), seeds, plant cells, and progeny of the same.

The term “non-conventional yeast” herein refers to any yeast that is not a Saccharomyces (e.g., S. cerevisiae) or Schizosaccharomyces yeast species. (see “Non-Conventional Yeasts in Genetics, Biochemistry and Biotechnology: Practical Protocols”, K. Wolf, K. D. Breunig, G. Barth, Eds., Springer-Verlag, Berlin, Germany, 2003).

The term “crossed” or “cross” or “crossing” in the context of this disclosure means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

The term “introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene, a modified (mutated or edited) native allele, or a selected allele of a marker or QTL.

The term “isoline” is a comparative term, and references organisms that are genetically identical, but differ in treatment. In one example, two genetically identical maize plant embryos may be separated into two different groups, one receiving a treatment (such as the introduction of a CRISPR-Cas effector endonuclease) and one control that does not receive such treatment. Any phenotypic differences between the two groups may thus be attributed solely to the treatment and not to any inherency of the plant's endogenous genetic makeup.

“Introducing” is intended to mean presenting to a target, such as a cell or organism, a polynucleotide or polypeptide or polynucleotide-protein complex, in such a manner that the component(s) gains access to the interior of a cell of the organism or to the cell itself

A “polynucleotide of interest” includes any nucleotide sequence encoding a protein or polypeptide that improves desirability of a host cell, plant, or crops. In some aspects, this includes, a trait of agronomic interest. Polynucleotides of interest: include, but are not limited to: markers, phenotypic characteristics, polynucleotides encoding important traits for agronomics (“traits of agronomic importance” or “traits of agronomic interest”): herbicide-resistance, insecticidal resistance, disease resistance, nematode resistance, herbicide resistance, microbial resistance, fungal resistance, viral resistance, fertility or sterility, grain characteristics, commercial products, phenotypic marker, or any other trait of agronomic or commercial importance. A polynucleotide of interest may additionally be utilized in either the sense or anti-sense orientation. Further, more than one polynucleotide of interest may be utilized together, or “stacked”, to provide additional benefit.

A “complex trait locus” includes a genomic locus that has multiple transgenes genetically linked to each other.

The compositions and methods herein may provide for an improved “agronomic trait” or “trait of agronomic importance” or “trait of agronomic interest” to a plant, which may include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein.

“Agronomic trait potential” is intended to mean a capability of a plant element for exhibiting a phenotype, preferably an improved agronomic trait, at some point during its life cycle, or conveying said phenotype to another plant element with which it is associated in the same plant.

The terms “decreased,” “fewer,” “slower” and “increased” “faster” “enhanced” “greater” as used herein refers to a decrease or increase in a characteristic of the modified plant element or resulting plant compared to an unmodified plant element or resulting plant. For example, a decrease in a characteristic may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400%) or more lower than the untreated control and an increase may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400% or more higher than the untreated control.

As used herein, the term “before”, in reference to a sequence position, refers to an occurrence of one sequence upstream, or 5′, to another sequence.

The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” or “umole” mean micromole(s), “g” means gram(s), “μg” or “ug” means microgram(s), “ng” means nanogram(s), “U” means unit(s), “bp” means base pair(s) and “kb” means kilobase(s).

### Classification of CRISPR-Cas Systems

CRISPR-Cas systems have been classified according to sequence and structural analysis of components. Multiple CRISPR/Cas systems have been described including Class 1 systems, with multisubunit effector complexes (comprising type I, type III, and type IV), and Class 2 systems, with single protein effectors (comprising type II, type V, and type VI) (Makarova et al. 2015, Nature Reviews Microbiology Vol. 13:1-15; Zetsche et al., 2015, Cell 163, 1-13; Shmakov et al., 2015, Molecular Cell 60, 1-13; Haft et al., 2005, Computational Biology, PLoS Comput Biol 1(6): e60; and Koonin et al. 2017, Curr Opinion Microbiology 37:67-78).

### CRISPR-Cas System Components

### Cas Proteins

A number of proteins may be encoded in the CRISPR cas operon, including those involved in adaptation (spacer insertion), interference (effector module target binding, target nicking or cleavage—e.g. endonuclease activity), expression (pre-crRNA processing), regulation, or other.

Two proteins, Casl and Cas2, are conserved among many CRISPR systems (for example, as described in Koonin et al., Curr Opinion Microbiology 37:67-78, 2017). Casl is a metal-dependent DNA-specific endonuclease that produces double-stranded DNA fragments. In some systems Cas1 forms a stable complex with Cas2, which is essential to spacer acquisition and insertion for CRISPR systems (Nunez et al., Nature Str Mol Biol 21:528-534, 2014).

A number of other proteins have been identified across different systems, including Cas4 (which may have similarity to a RecB nuclease) and is thought to play a role in the capture of new viral DNA sequences for incorporation into the CRISPR array (Zhang et al., PLOS One 7(10):e47232, 2012).

Some proteins may encompass a plurality of functions. For example, Cas9, the signature protein of Class 2 type II systems, has been demonstrated to be involved in pre-crRNA processing, target binding, as well as target cleavage.

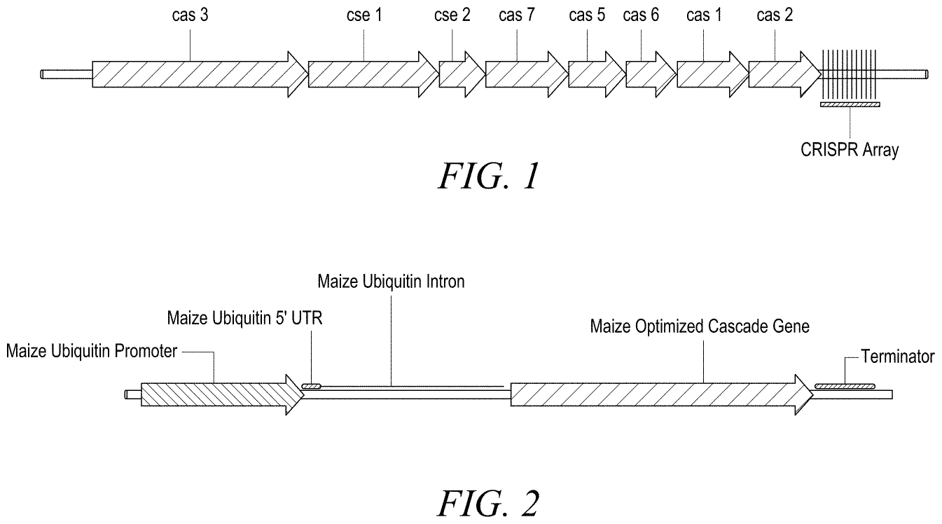

In the Type I-E system, the cas operon comprises, as shown in FIG. 1, genes encoding: an endonuclease (Cas3), five proteins that assemble to form the cascade (Cse1, Cse2, Cas7, Cas5, and Cas6), Cas1, and Cas2. The CRISPR locus further comprises a CRISPR array comprising a series of repeats and spacers. Within cascade, Cas5, Cas7, Cse1, and Cse2 are involved in DNA target recognition. Cas 6 is involved in CRISPR RNA maturation. Equivalent terminology for Cse1 includes Cas8, Cas8e, and CasA.

In some aspects, Cas5 is a polypeptide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:10. In some aspects, Cas5 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:2.

In some aspects, Cas6 is a polypeptide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:11. In some aspects, Cas6 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:3.

In some aspects, Cas7 is a polypeptide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:12. In some aspects, Cas7 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:4.

In some aspects, Cse1 is a polypeptide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:13. In some aspects, Cse1 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:5.

In some aspects, Cse2 is a polypeptide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:14. In some aspects, Cse2 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:6.

In some aspects, Casl is a polypeptide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:16. In some aspects, Casl is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:8.

In some aspects, Cas2 is a polypeptide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:17. In some aspects, Cas2 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:9.

### Cas Endonucleases and Effectors

Endonucleases are enzymes that cleave the phosphodiester bond within a polynucleotide chain, and include restriction endonucleases that cleave DNA at specific sites without damaging the bases. Examples of endonucleases include restriction endonucleases, meganucleases, TAL effector nucleases (TALENs), zinc finger nucleases, and Cas (CRISPR-associated) effector endonucleases.

Cas endonucleases, either as single effector proteins or in an effector complex with other components, unwind the DNA duplex at the target sequence and optionally cleave at least one DNA strand, as mediated by recognition of the target sequence by a polynucleotide (such as, but not limited to, a crRNA or guide RNA) that is in complex with the Cas effector protein. Such recognition and cutting of a target sequence by a Cas endonuclease typically occurs if the correct protospacer-adjacent motif (PAM) is located at or adjacent to the 3′ end of the DNA target sequence. Alternatively, a Cas endonuclease herein may lack DNA cleavage or nicking activity, but can still specifically bind to a DNA target sequence when complexed with a suitable RNA component.

Cas endonucleases may occur as individual effectors (Class 2 CRISPR systems) or as part of larger effector complexes (Class I CRISPR systems).

Cas endonucleases that have been described include, but are not limited to, for example: Cas3 (a feature of Class 1 type I systems), Cas9 (a feature of Class 2 type II systems) and Cas12 (Cpf1) (a feature of Class 2 type V systems).

Cas3 (and its variants Cas3′ and Cas3″) functions as a single-stranded DNA nuclease (HD domain) and an ATP-dependent helicase. A variant of the Cas3 endonuclease can be obtained by disabling the functional activity of one or both domains of the Cas3 endonuclease polypeptide. Disabling the ATPase dependent helicase activity (by or deletion, knockout of the Cas3-helicase domain or through mutagenesis of critical residues or by assembling the reaction in the absence of ATP as described previously (Sinkunas, T. et al., 2013, EMBO J. 32:385-394) can convert the cleavage ready cascade comprising the modified Cas3 endonuclease into a nickase (as the HD domain is still functional). Disabling the HD endonuclease activity can be accomplished by domain by any method known in the art, such as but not limited to, mutagenesis of critical residues of the HD domain, can convert the cleavage ready cascade comprising the modified Cas3 endonuclease into a helicase. Disabling the both the Cas helicase and Cas3 HD endonuclease activity can be accomplished by any method known in the art, such as but not limited to, mutagenesis of critical residues of both the helicase and HD domains, can convert the cleavage ready cascade comprising the modified Cas3 endonuclease into a binder protein that binds to a target sequence.

An “engineered Cas3” endonuclease refers a Cas3 protein that has been modified from its original sequence or structure, for example to function as a nickase that may nick one or both strands of a double stranded polynucleotide. When nicking occurs on both strands, the engineered Cas3 endonuclease creates and effective double-strand break (DSB) by virtue of the cooperative nature of nicks on opposite strands: two targets on opposite DNA strands provide opportunity for an effective DSB, that may result in a sticky end 5′ overhang, a sticky end 3′ overhang, a blunt end cut, or a blunt end cut with sticky end overhang(s).

In some aspects, Cas3 is a polypeptide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 175, at least 175, between 175 and 195, or greater than 195 contiguous amino acids of SEQ ID NO:15. In some aspects, Cas3 is a polypeptide encoded by a polynucleotide sequence that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, or greater than 450 contiguous nucleotides SEQ ID NO:7.

Type I-E Cas endonucleases and effector proteins can be used for targeted genome editing (via simplex and multiplex double-strand breaks and nicks) and targeted genome regulation (via tethering of epigenetic effector domains to either the Cas protein or sgRNA. A Cas endonuclease can also be engineered to function as an RNA-guided recombinase, and via RNA tethers could serve as a scaffold for the assembly of multiprotein and nucleic acid complexes (Mali et al., 2013, Nature Methods Vol. 10: 957-963).

A Type I-E Cascade, Cascade protein, Cas endonuclease, effector protein, or functional fragment thereof, for use in the disclosed methods, can be isolated from a native source, or from, a recombinant source where the genetically modified host cell is modified to express the nucleic acid sequence encoding the protein. Alternatively, the Cas protein can be produced using cell free protein expression systems, or be synthetically produced. Effector Cas nucleases may be isolated and introduced into a heterologous cell, or may be modified from its native form to exhibit a different type or magnitude of activity than what it would exhibit in its native source. Such modifications include but are not limited to: fragments, variants, substitutions, deletions, chemical alterations, and insertions.

Fragments and variants of Cas endonucleases and Cas effector proteins can be obtained via methods such as site-directed mutagenesis and synthetic construction. Methods for measuring endonuclease activity are well known in the art such as, but not limiting to, WO2013166113 published 7 Nov. 2013, WO2016186953 published 24 Nov. 2016, and WO2016186946 published 24 Nov. 2016.

The Cas endonuclease can comprise a modified form of the Cas polypeptide. The modified form of the Cas polypeptide can include an amino acid change (e.g., deletion, insertion, chemical alteration, or substitution) that reduces the naturally-occurring nuclease activity of the Cas protein. For example, in some instances, the modified form of the Cas protein has less than 50%, less than 40%, less than 30%, less than 20%, less than 10%, less than 5%, or less than 1% of the nuclease activity of the corresponding wild-type Cas polypeptide (US20140068797 published 6 Mar. 2014). In some cases, the modified form of the Cas polypeptide has no substantial nuclease activity and is referred to as catalytically “inactivated Cas” or “deactivated Cas (dCas).” An inactivated Cas/deactivated Cas includes a deactivated Cas endonuclease (dCas). A catalytically inactive Cas effector protein can be fused to a heterologous sequence to induce or modify activity.

A Cas endonuclease can be part of a fusion protein comprising one or more heterologous protein domains (e.g., 1, 2, 3, or more domains in addition to the Cas protein). Such a fusion protein may comprise any additional protein sequence, and optionally a linker sequence between any two domains, such as between Cas and a first heterologous domain. Examples of protein domains that may be fused to a Cas protein herein include, without limitation, epitope tags (e.g., histidine [His], V5, FLAG, influenza hemagglutinin [HA], myc, VSV-G, thioredoxin [Trx]), reporters (e.g., glutathione-5-transferase [GST], horseradish peroxidase [HRP], chloramphenicol acetyltransferase [CAT], beta-galactosidase, beta-glucuronidase [GUS], luciferase, green fluorescent protein [GFP], HcRed, DsRed, cyan fluorescent protein [CFP], yellow fluorescent protein [YFP], blue fluorescent protein [BFP]), and domains having one or more of the following activities: methylase activity, demethylase activity, transcription activation activity (e.g., VP16 or VP64), transcription repression activity, transcription release factor activity, histone modification activity, RNA cleavage activity and nucleic acid binding activity. A Cas protein can also be in fusion with a protein that binds DNA molecules or other molecules, such as maltose binding protein (MBP), S-tag, Lex A DNA binding domain (DBD), GAL4A DNA binding domain, and herpes simplex virus (HSV) VP16.

A catalytically active and/or inactive Cas endonuclease can be fused to a heterologous sequence (US20140068797 published 06 March 2014). Suitable fusion partners include, but are not limited to, a polypeptide that provides an activity that indirectly increases transcription by acting directly on the target DNA or on a polypeptide (e.g., a histone or other DNA-binding protein) associated with the target DNA. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.).

The Cas proteins described herein can be expressed and purified by methods known in the art, for example as described in WO/2016/186953 published 24 Nov. 2016.

A Cas effector protein can comprise a heterologous nuclear localization sequence (NLS). A heterologous NLS amino acid sequence herein may be of sufficient strength to drive accumulation of a Cas protein in a detectable amount in the nucleus of a yeast cell herein, for example. An NLS may comprise one (monopartite) or more (e.g., bipartite) short sequences (e.g., 2 to 20 residues) of basic, positively charged residues (e.g., lysine and/or arginine), and can be located anywhere in a Cas amino acid sequence but such that it is exposed on the protein surface. An NLS may be operably linked to the N-terminus or C-terminus of a Cas protein herein, for example. Two or more NLS sequences can be linked to a Cas protein, for example, such as on both the N- and C-termini of a Cas protein. The Cas endonuclease gene can be operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region. Non-limiting examples of suitable NLS sequences herein include those disclosed in U.S. Pat. Nos. 6,660,830 and 7,309,576.

### Guide Polynucleotides

The guide polynucleotide enables target recognition, binding, and optionally cleavage by the Cas endonuclease, and can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (US20150082478 published 19 Mar. 2015 and US20150059010 published 26 Feb. 2015). A guide polynucleotide may be engineered or synthetic.

The guide polynucleotide includes a chimeric non-naturally occurring guide RNA comprising regions that are not found together in nature (i.e., they are heterologous with each other). For example, a chimeric non-naturally occurring guide RNA comprising a first nucleotide sequence domain (referred to as Variable Targeting domain or VT domain) that can hybridize to a nucleotide sequence in a target DNA, linked to a second nucleotide sequence that can recognize the Cas endonuclease, such that the first and second nucleotide sequence are not found linked together in nature.

In one aspect, the guide polynucleotide is a guide polynucleotide capable of forming a ribonucleoprotein complex, wherein said guide polynucleotide comprises a first nucleotide sequence domain that is complementary to a nucleotide sequence in a target DNA, and a second nucleotide sequence domain that interacts with said Cas endonuclease polypeptide.

In one aspect, the guide polynucleotide is a guide polynucleotide described herein, wherein the first nucleotide sequence and the second nucleotide sequence domain is selected from the group consisting of a DNA sequence, a RNA sequence, and a combination thereof

In one aspect, the guide polynucleotide is a guide polynucleotide described herein, wherein the first nucleotide sequence and the second nucleotide sequence domain is selected from the group consisting of RNA backbone modifications that enhance stability, DNA backbone modifications that enhance stability, and a combination thereof (see Kanasty et al., 2013, Common RNA-backbone modifications, Nature Materials 12:976-977; US20150082478 published 19 Mar. 2015 and US20150059010 published 26 Feb. 2015)

The guide RNA includes a dual molecule comprising a chimeric non-naturally occurring crRNA linked to at least one tracrRNA. A chimeric non-naturally occurring crRNA includes a crRNA that comprises regions that are not found together in nature (i.e., they are heterologous with each other. For example, a crRNA comprising a first nucleotide sequence domain (referred to as Variable Targeting domain or VT domain) that can hybridize to a nucleotide sequence in a target DNA, linked to a second nucleotide sequence (also referred to as a tracr mate sequence) such that the first and second sequence are not found linked together in nature.

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide

The VT domain and /or the CER domain of a single guide polynucleotide can comprise a RNA sequence, a DNA sequence, or a RNA-DNA-combination sequence. The single guide polynucleotide being comprised of sequences from the crNucleotide and the tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double-strand break) the target site. (US20150082478 published 19 Mar. 2015 and US20150059010 published 26 Feb. 2015).

A chimeric non-naturally occurring single guide RNA (sgRNA) includes a sgRNA that comprises regions that are not found together in nature (i.e., they are heterologous with each other. For example, a sgRNA comprising a first nucleotide sequence domain (referred to as Variable Targeting domain or VT domain) that can hybridize to a nucleotide sequence in a target DNA linked to a second nucleotide sequence (also referred to as a tracr mate sequence) that are not found linked together in nature.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide (also referred to as “loop”) can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetraloop sequence, such as, but not limiting to a GAAA tetraloop sequence.

The guide polynucleotide can be produced by any method known in the art, including chemically synthesizing guide polynucleotides (such as but not limiting to Hendel et al. 2015, Nature Biotechnology 33, 985-989), in vitro generated guide polynucleotides, and/or self-splicing guide RNAs (such as but not limited to Xie et al. 2015, PNAS 112:3570-3575).

### Protospacer Adjacent Motif (PAM)

A “protospacer adjacent motif” (PAM) herein refers to a short nucleotide sequence adjacent to a target sequence (protospacer) that can be recognized (targeted) by a guide polynucleotide/Cas endonuclease system. Many Cas endonucleases require a PAM sequence to successfully recognize a target DNA sequence. The sequence and length of a PAM may differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long. Some Cas endonucleases may not require a PAM sequence.

A “randomized PAM” and “randomized protospacer adjacent motif” are used interchangeably herein, and refer to a random DNA sequence adjacent to a target sequence (protospacer) that is recognized (targeted) by a guide polynucleotide/Cas endonuclease system. The randomized PAM sequence can be of any length but is typically 1, 2, 3, 4, 5, 6, 7, 8, 9, 10, 11, 12, 13, 14, 15, 16, 17, 18, 19 or 20 nucleotides long. A randomized nucleotide includes anyone of the nucleotides A, C, G or T.

### Guide Polynucleotide/Cas Endonuclease Complexes

A guide polynucleotide/Cascade complex described herein is capable of recognizing, binding to, and optionally nicking, unwinding, or cleaving all or part of a target sequence.

A guide polynucleotide/Cascade complex that can cleave both strands of a DNA target sequence typically comprises a Cas protein that has all of its endonuclease domains in a functional state (e.g., wild type endonuclease domains or variants thereof retaining some or all activity in each endonuclease domain). Thus, a wild type Cas protein (e.g., a Cas protein disclosed herein), or a variant thereof retaining some or all activity in each endonuclease domain of the Cas protein, is a suitable example of a Cas endonuclease that can cleave both strands of a DNA target sequence.

A guide polynucleotide/Cascade complex that can cleave one strand of a DNA target sequence can be characterized herein as having nickase activity (e.g., partial cleaving capability). A Cas nickase typically comprises one functional endonuclease domain that allows the Cas to cleave only one strand (i.e., make a nick) of a DNA target sequence. A pair of Cas nickases can be used to increase the specificity of DNA targeting. In general, this can be done by providing two Cas nickases that, by virtue of being associated with RNA components with different guide sequences, target and nick nearby DNA sequences on opposite strands in the region for desired targeting. Such nearby cleavage of each DNA strand creates a double-strand break (i.e., a DSB with single-stranded overhangs), which is then recognized as a substrate for non-homologous-end-joining, NHEJ (prone to imperfect repair leading to mutations) or homologous recombination, HR. Each nick in these embodiments can be at least about 5, between 5 and 10, at least 10, between 10 and 15, at least 15, between 15 and 20, at least 20, between 20 and 30, at least 30, between 30 and 40, at least 40, between 40 and 50, at least 50, between 50 and 60, at least 60, between 60 and 70, at least 70, between 70 and 80, at least 80, between 80 and 90, at least 90, between 90 and 100, or 100 or greater (or any integer between 5 and 100) bases apart from each other, for example. One or two Cas nickase proteins herein can be used in a Cas nickase pair.

A guide polynucleotide/Cascade complex in certain embodiments can bind to a DNA target site sequence, but does not cleave any strand at the target site sequence. Such a complex may comprise a Cas protein in which all of its nuclease domains are mutant, dysfunctional. A Cas protein herein that binds, but does not cleave, a target DNA sequence can be used to modulate gene expression, for example, in which case the Cas protein could be fused with a transcription factor (or portion thereof) (e.g., a repressor or activator, such as any of those disclosed herein).

In one aspect, the guide polynucleotide/Cas endonuclease complex described herein is a ribonucleoprotein complex, wherein said Cas endonuclease is covalently or non-covalently linked, or assembled to at least one cascade protein subunit, or functional fragment thereof

In one embodiment of the disclosure, the guide polynucleotide/Cascade complex is a guide polynucleotide/Cascade complex comprising at least one guide polynucleotide and at least one Cas endonuclease polypeptide, wherein said Cas endonuclease polypeptide comprises at least one protein subunit of a cascade, or a functional fragment thereof, wherein said guide polynucleotide is a chimeric non-naturally occurring guide polynucleotide, wherein said guide polynucleotide/Cas endonuclease complex is capable of recognizing, binding to, and optionally nicking, unwinding, or cleaving all or part of a target sequence.

The Cas effector protein can be a Cas3 effector protein as disclosed herein.

In one embodiment of the disclosure, the guide polynucleotide/Cas effector complex is a guide polynucleotide/Cas effector protein complex (PGEN) comprising at least one guide polynucleotide and a Cas3 effector protein, wherein said guide polynucleotide/Cas effector protein complex is capable of recognizing, binding to, and optionally nicking, unwinding, or cleaving all or part of a target sequence.

The PGEN can be a guide polynucleotide/Cas effector protein complex, wherein said Cas effector protein further comprises one copy or multiple copies of at least one protein subunit, or a functional fragment thereof, of a cascade. In some embodiments, said cascade protein is selected from the group consisting of a Casl protein subunit and, optionally, a Cas2 protein subunit, and any combination thereof. The PGEN can be a guide polynucleotide/Cas effector protein complex, wherein said Cas effector protein further comprises at least Cas1 and, optionally, Cas2.

In one aspect, the guide polynucleotide/Cas effector protein complex (PGEN) described herein is a PGEN, wherein said Cas effector protein is covalently or non-covalently linked to at least one cascade protein subunit, or functional fragment thereof. The PGEN can be a guide polynucleotide/Cas effector protein complex, wherein said Cas effector protein polypeptide is covalently or non-covalently linked, or assembled to one copy or multiple copies of at least one protein subunit, or a functional fragment thereof, of a Cas1 protein subunit, and optionally Cas2, and any combination thereof, effectively forming a cleavage ready cascade. The PGEN can be a guide polynucleotide/Cas effector protein complex, wherein said Cas effector protein is covalently or non-covalently linked or assembled to Cas1, and optionally Cas2,

Any component of the guide polynucleotide/Cas effector protein complex, the guide polynucleotide/Cas effector protein complex itself, as well as the polynucleotide modification template(s) and/or donor DNA(s), can be introduced into a heterologous cell or organism by any method known in the art.

### Recombinant Constructs for Transformation of Cells

The disclosed guide polynucleotides, Cas endonucleases, polynucleotide modification templates, donor DNAs, guide polynucleotide/Cas endonuclease systems disclosed herein, and any one combination thereof, optionally further comprising one or more polynucleotide(s) of interest, can be introduced into a cell. Cells include, but are not limited to, human, non-human, animal, bacterial, fungal, insect, yeast, non-conventional yeast, and plant cells as well as plants and seeds produced by the methods described herein.

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, N.Y. (1989). Transformation methods are well known to those skilled in the art and are described infra.

Vectors and constructs include circular plasmids, and linear polynucleotides, comprising a polynucleotide of interest and optionally other components including linkers, adapters, regulatory or analysis. In some examples a recognition site and/or target site can be comprised within an intron, coding sequence, 5′ UTRs, 3′ UTRs, and/or regulatory regions.

Components for Expression and Utilization of Novel CRISPR-Cas Systems in Prokaryotic and Eukaryotic cells

The invention further provides expression constructs for expressing in a prokaryotic or eukaryotic cell/organism a guide RNA/Cas system that is capable of recognizing, binding to, and optionally nicking, unwinding, or cleaving all or part of a target sequence.

In one embodiment, the expression constructs of the disclosure comprise a promoter operably linked to a nucleotide sequence encoding a Cas gene (or plant optimized, including a Cas endonuclease gene described herein) and a promoter operably linked to a guide RNA of the present disclosure. The promoter is capable of driving expression of an operably linked nucleotide sequence in a prokaryotic or eukaryotic cell/organism.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking , a modification or sequence that provides a binding site for proteins , a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

A method of expressing RNA components such as gRNA in eukaryotic cells for performing Cas9-mediated DNA targeting has been to use RNA polymerase III (Pol III) promoters, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3:e161). This strategy has been successfully applied in cells of several different species including maize and soybean (US20150082478 published 19 Mar. 2015). Methods for expressing RNA components that do not have a 5′ cap have been described (WO2016/025131 published 18 Feb. 2016).

Various methods and compositions can be employed to obtain a cell or organism having a polynucleotide of interest inserted in a target site for a Cas endonuclease. Such methods can employ homologous recombination (HR) to provide integration of the polynucleotide of interest at the target site. In one method described herein, a polynucleotide of interest is introduced into the organism cell via a donor DNA construct.

The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10: 957-963).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity at least of about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, between 98% and 99%, 99%, between 99% and 100%, or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, N.Y.).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some instances the regions of homology share significant sequence homology to the genomic sequence immediately flanking the target site, it is recognized that the regions of homology can be designed to have sufficient homology to regions that may be further 5′ or 3′ to the target site. The regions of homology can also have homology with a fragment of the target site along with downstream genomic regions

In one embodiment, the first region of homology further comprises a first fragment of the target site and the second region of homology comprises a second fragment of the target site, wherein the first and second fragments are dissimilar.

### Polynucleotides of Interest

General categories of polynucleotides of interest include those that confer a trait of interest or importance to the cell comprising the polynucleotide, an organism comprising the polynucleotide, or to a progeny of the cell or organism. In some embodiments, the polynucleotide conferring the trait or characteristic of interest is inherited by the progeny of the cell or organism. In some embodiments, the polynucleotide itself is not transferred to the progeny but the characteristic that it imparts is (e.g., a genomic modification effected by the polynucleotide is retained).

Polynucleotides of interest include, for example but not limited to, markers, genes of interest involved in information or genomic modification (such as zinc fingers or cas endonucleases), those involved in communication (such as kinases), and those involved in housekeeping (such as heat shock proteins).

Other traits of interest or importance include phenotypes that enable selection (markers). In some aspects, the trait of interest or importance includes those that provide an improvement in a cellular or organism characteristic, for example but not limited to: selectable marker resistance, disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein composition, altered oil composition, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, improved fertility, improved fecundity, improved environmental tolerance, improved vigor, improved disease resistance, improved disease tolerance, improved tolerance to a heterologous molecule, improved fitness, improved physical characteristic, greater mass, increased production of a biochemical molecule, decreased production of a biochemical molecule, upregulation of a gene, downregulation of a gene, upregulation of a biochemical pathway, downregulation of a biochemical pathway, stimulation of cell reproduction, and suppression of cell reproduction.

Polynucleotides of interest are further described herein and include polynucleotides reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for genetic engineering will change accordingly.

Polynucleotides of agronomic interest also include, but are not limited to, genes involved in traits of agronomic interest (a.k.a. “traits of agronomic importance”) such as but not limited to: crop yield, grain quality, crop nutrient content, starch and carbohydrate quality and quantity as well as those affecting kernel size, sucrose loading, protein quality and quantity, nitrogen fixation and/or utilization, fatty acid and oil composition, genes encoding proteins conferring resistance to abiotic stress (such as drought, nitrogen, temperature, salinity, toxic metals or trace elements, or those conferring resistance to toxins such as pesticides and herbicides), genes encoding proteins conferring resistance to biotic stress (such as attacks by fungi, viruses, bacteria, insects, and nematodes, and development of diseases associated with these organisms).

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389.

Polynucleotide sequences of interest may encode proteins involved in providing disease or pest resistance. By “disease resistance” or “pest resistance” is intended that the plants avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonisin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like. Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS, also referred to as acetohydroxyacid synthase, AHAS), in particular the sulfonylurea (UK: sulphonylurea) type herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g, the HPPD gene) or other such genes known in the art. See, for example, U.S. Pat. Nos. 7,626,077, 5,310,667, 5,866,775, 6,225,114, 6,248,876, 7,169,970, 6,867,293, and 9,187,762.

Furthermore, it is recognized that the polynucleotide of interest may also comprise anti sense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the polynucleotide of interest may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See U.S. Pat. Nos. 5,283,184 and 5,034,323.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that comprises it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as sulphonylureas, glufosinate ammonium, bromoxynil, imidazolinones, and 2,4-dichlorophenoxyacetate (2,4-D). See for example, Acetolactase synthase (ALS) for resistance to sulfonylureas, imidazolinones, triazolopyrimidine sulfonamides, pyrimidinylsalicylates and sulphonylaminocarbonyl-triazolinones (Shaner and Singh, 1997, Herbicide Activity: Toxicol Biochem Mol Biol 69-110); glyphosate resistant 5-enolpyruvylshikimate-3-phosphate (EPSPS) (Saroha et al. 1998, J. Plant Biochemistry & Biotechnology Vol 7:65-72);

Polynucleotides of interest includes genes that can be stacked or used in combination with other traits, such as but not limited to herbicide resistance or any other trait described herein. Polynucleotides of interest and/or traits can be stacked together in a complex trait locus as described in US20130263324 published 3 Oct. 2013 and in WO/2013/112686, published 1 Aug. 2013.

A polypeptide of interest includes any protein or polypeptide that is encoded by a polynucleotide of interest described herein.

Further provided are methods for identifying at least one plant cell, comprising in its genome, a polynucleotide of interest integrated at the target site. A variety of methods are available for identifying those plant cells with insertion into the genome at or near to the target site. Such methods can be viewed as directly analyzing a target sequence to detect any change in the target sequence, including but not limited to PCR methods, sequencing methods, nuclease digestion, Southern blots, and any combination thereof. See, for example, US20090133152 published 21 May 2009. The method also comprises recovering a plant from the plant cell comprising a polynucleotide of interest integrated into its genome. The plant may be sterile or fertile. It is recognized that any polynucleotide of interest can be provided, integrated into the plant genome at the target site, and expressed in a plant.

### Optimization of Sequences for Expression in Plants

Methods are available in the art for synthesizing plant-preferred genes. See, for example, U.S. Pat. Nos. 5,380,831, and 5,436,391, and Murray et al. (1989) Nucleic Acids Res. 17:477-498. Additional sequence modifications are known to enhance gene expression in a plant host. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures. Thus, “a plant-optimized nucleotide sequence” of the present disclosure comprises one or more of such sequence modifications.

### Expression Elements

Any polynucleotide encoding a Cas protein or other CRISPR system component disclosed herein may be functionally linked to a heterologous expression element, to facilitate transcription or regulation in a host cell. Such expression elements include but are not limited to: promoter, leader, intron, and terminator. Expression elements may be “minimal”—meaning a shorter sequence derived from a native source, that still functions as an expression regulator or modifier. Alternatively, an expression element may be “optimized”—meaning that its polynucleotide sequence has been altered from its native state in order to function with a more desirable characteristic in a particular host cell (for example, but not limited to, a bacterial promoter may be “ ” to improve its expression in corn plants). Alternatively, an expression element may be “synthetic”—meaning that it is designed in silico and synthesized for use in a host cell. Synthetic expression elements may be entirely synthetic, or partially synthetic (comprising a fragment of a naturally-occurring polynucleotide sequence).

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters” if the promoters direct RNA synthesis preferably in certain tissues but also in other tissues at reduced levels.

A plant promoter includes a promoter capable of initiating transcription in a plant cell. For a review of plant promoters, see, Potenza et al., 2004, In vitro Cell Dev Biol 40:1-22; Porto et al., 2014, Molecular Biotechnology (2014), 56(1), 38-49.

New promoters of various types useful in plant cells are constantly being discovered; numerous examples may be found in the compilation by Okamuro and Goldberg, (1989) In The Biochemistry of Plants, Vol. 115, Stumpf and Conn, eds (New York, N.Y.: Academic Press), pp. 1-82.

Modification of Genomes with Novel CRISPR-Cas System Components

As described herein, a guided Cascade can recognize, bind to a DNA target sequence and introduce a single strand (nick) or double-strand break. Once a single or double-strand break is induced in the DNA, the cell's DNA repair mechanism is activated to repair the break. Error-prone DNA repair mechanisms can produce mutations at double-strand break sites.

The most common repair mechanism to bring the broken ends together is the nonhomologous end-joining (NHEJ) pathway (Bleuyard et al., (2006) DNA Repair 5:1-12). The structural integrity of chromosomes is typically preserved by the repair, but deletions, insertions, or other rearrangements (such as chromosomal translocations) are possible (Siebert and Puchta, 2002, Plant Cell 14:1121-31; Pacher et al., 2007, Genetics 175:21-9). Other pathways such as MMEJ and alt-NHEJ are also contemplated.

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. PNAS (0027-8424), 111 (10), p. E924-E932).

### Gene Targeting

The guide polynucleotide/Cas systems described herein can be used for gene targeting.

In general, DNA targeting can be performed by cleaving one or both strands at a specific polynucleotide sequence in a cell with a Cas protein associated with a suitable polynucleotide component. Once a single or double-strand break is induced in the DNA, the cell's DNA repair mechanism is activated to repair the break via nonhomologous end-joining (NHEJ) or Homology-Directed Repair (HDR) processes which can lead to modifications at the target site.

The length of the DNA sequence at the target site can vary, and includes, for example, target sites that are at least 12, 13, 14, 15, 16, 17, 18, 19, 20, 21, 22, 23, 24, 25, 26, 27, 28, 29, 30, or more than 30 nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease.

Assays to measure the single or double-strand break of a target site by an endonuclease are known in the art and generally measure the overall activity and specificity of the agent on DNA substrates comprising recognition sites.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide a guide polynucleotide/Cas endonuclease complex to a unique DNA target site.

### Gene Editing

The process for editing a genomic sequence combining DSB and modification templates generally comprises: introducing into a host cell a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB. Genome editing using DSB-inducing agents, such as Cas-gRNA complexes, has been described, for example in US20150082478 published on 19 Mar. 2015, WO2015026886 published on 26 Feb. 2015, WO2016007347 published 14 Jan. 2016, and WO/2016/025131 published on 18 Feb. 2016.

Some uses for guide RNA/Cas endonuclease systems have been described (see for example: US20150082478 A1 published 19 Mar. 2015, WO2015026886 published 26 Feb. 2015, and US20150059010 published 26 Feb. 2015) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, chemical alterations of a nucleotide such as by the addition or deletion or substitution of one or more atoms, amino acid and/or protein fusions, and gene silencing by expressing an inverted repeat into a gene of interest.

Proteins may be altered in various ways including amino acid substitutions, deletions, truncations, insertions, and chemical alterations of one or more amino acids such as by the addition or deletion or substitution of one or more atoms. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates comprising target sites.

Described herein are methods for genome editing with Cleavage Ready cascade (crCascade) Complexes. Following characterization of the guide RNA and PAM sequence, components of the cleavage ready cascade (crCascade) complex and associated CRISPR RNA (crRNA) may be utilized to modify chromosomal DNA in other organisms including plants. To facilitate optimal expression and nuclear localization (for eukaryotic cells), the genes comprising the crCascade may be optimized as described in W02016186953 published 24 November 2016, and then delivered into cells as DNA expression cassettes by methods known in the art. The components necessary to comprise an active crCascade complex may also be delivered as RNA with or without modifications that protect the RNA from degradation or as mRNA capped or uncapped (Zhang, Y. et al., 2016, Nat. Commun. 7:12617) or Cas protein guide polynucleotide complexes (WO2017070032 published 27 Apr. 2017), or any combination thereof. Additionally, a part or part(s) of the crCascade complex and crRNA may be expressed from a DNA construct while other components are delivered as RNA with or without modifications that protect the RNA from degradation or as mRNA capped or uncapped (Zhang et al. 2016 Nat. Commun. 7:12617) or Cas protein guide polynucleotide complexes (WO2017070032 published 27 April 2017) or any combination thereof. To produce crRNAs in-vivo, tRNA derived elements may also be used to recruit endogenous RNAses to cleave crRNA transcripts into mature forms capable of guiding the crCascade complex to its DNA target site, as described, for example, in WO2017105991 published 22 Jun. 2017. crCascade nickase complexes may be utilized separately or concertedly to generate a single or multiple DNA nicks on one or both DNA strands. Furthermore, the cleavage activity of the Cas endonuclease may be deactivated by altering key catalytic residues in its cleavage domain (Sinkunas, T. et al., 2013, EMBO J. 32:385-394) resulting in a RNA guided helicase that may be used to enhance homology directed repair, induce transcriptional activation, or remodel local DNA structures. Moreover, the activity of the Cas cleavage and helicase domains may both be knocked-out and used in combination with other DNA cutting, DNA nicking, DNA binding, transcriptional activation, transcriptional repression, DNA remodeling, DNA deamination, DNA unwinding, DNA recombination enhancing, DNA integration, DNA inversion, and DNA repair agents.

The transcriptional direction of the tracrRNA for the CRISPR-Cas system (if present) and other components of the CRISPR-Cas system (such as variable targeting domain, crRNA repeat, loop, anti-repeat) can be deduced as described in WO2016186946 published 24 Nov. 2016, and WO2016186953 published 24 Nov. 2016.

As described herein, once the appropriate guide RNA requirement is established, the PAM preferences for each new system disclosed herein may be examined. If the cleavage ready cascade (crCascade) complex results in degradation of the randomized PAM library, the crCascade complex can be converted into a nickase by disabling the ATPase dependent helicase activity either through mutagenesis of critical residues or by assembling the reaction in the absence of ATP as described previously (Sinkunas, T. et al., 2013, EMBO J. 32:385-394). Two regions of PAM randomization separated by two protospacer targets may be utilized to generate a double-stranded DNA break which may be captured and sequenced to examine the PAM sequences that support cleavage by the respective crCascade complex.

In one embodiment, the invention describes a method for modifying a target site in the genome of a cell, the method comprising introducing into a cell at least one PGEN described herein, and identifying at least one cell that has a modification at said target, wherein the modification at said target site is selected from the group consisting of (i) a replacement of at least one nucleotide, (ii) a deletion of at least one nucleotide, (iii) an insertion of at least one nucleotide, and (iv) any combination of (i)-(iii).

The nucleotide to be edited can be located within or outside a target site recognized and cleaved by a Cas endonuclease. In one embodiment, the at least one nucleotide modification is not a modification at a target site recognized and cleaved by a Cas endonuclease. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

A knock-out may be produced by an indel (insertion or deletion of nucleotide bases in a target DNA sequence through NHEJ), or by specific removal of sequence that reduces or completely destroys the function of sequence at or near the targeting site.

A guide polynucleotide/Cas endonuclease induced targeted mutation can occur in a nucleotide sequence that is located within or outside a genomic target site that is recognized and cleaved by the Cas endonuclease.

The method for editing a nucleotide sequence in the genome of a cell can be a method without the use of an exogenous selectable marker by restoring function to a non-functional gene product.

In one embodiment, the invention describes a method for modifying a target site in the genome of a cell, the method comprising introducing into a cell at least one PGEN described herein and at least one donor DNA, wherein said donor DNA comprises a polynucleotide of interest, and optionally, further comprising identifying at least one cell that said polynucleotide of interest integrated in or near said target site.

In one aspect, the methods disclosed herein may employ homologous recombination (HR) to provide integration of the polynucleotide of interest at the target site.

Various methods and compositions can be employed to produce a cell or organism having a polynucleotide of interest inserted in a target site via activity of a CRISPR-Cas system component described herein. In one method described herein, a polynucleotide of interest is introduced into the organism cell via a donor DNA construct. As used herein, “donor DNA” is a DNA construct that comprises a polynucleotide of interest to be inserted into the target site of a Cas endonuclease. The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10: 957-963).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, N.Y.).

Episomal DNA molecules can also be ligated into the double-strand break, for example, integration of T-DNAs into chromosomal double-strand breaks (Chilton and Que, (2003) Plant Physiol 133:956-65; Salomon and Puchta, (1998) EMBO J. 17:6086-95). Once the sequence around the double-strand breaks is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

In one embodiment, the disclosure comprises a method for editing a nucleotide sequence in the genome of a cell, the method comprising introducing into at least one PGEN described herein, and a polynucleotide modification template, wherein said polynucleotide modification template comprises at least one nucleotide modification of said nucleotide sequence, and optionally further comprising selecting at least one cell that comprises the edited nucleotide sequence.

The guide polynucleotide/Cas endonuclease system can be used in combination with at least one polynucleotide modification template to allow for editing (modification) of a genomic nucleotide sequence of interest. (See also US20150082478, published 19 Mar. 2015 and WO2015026886 published 26 Feb. 2015).

Polynucleotides of interest and/or traits can be stacked together in a complex trait locus as described in WO2012129373 published 27 Sep. 2012, and in WO2013112686, published 1 Aug. 2013. The guide polynucleotide/endonuclease system described herein provides for an efficient system to generate double-strand breaks and allows for traits to be stacked in a complex trait locus.

A guide polynucleotide/Cas system as described herein, mediating gene targeting, can be used in methods for directing heterologous gene insertion and/or for producing complex trait loci comprising multiple heterologous genes in a fashion similar as disclosed in WO2012129373 published 27 Sep. 2012, where instead of using a double-strand break inducing agent to introduce a gene of interest, a guide polynucleotide/Cas system as disclosed herein is used. By inserting independent transgenes within 0.1, 0.2, 0.3, 0.4, 0.5, 1.0, 2, or even 5 centimorgans (cM) from each other, the transgenes can be bred as a single genetic locus (see, for example, US20130263324 published 3 Oct. 2013 or WO2012129373 published 14 Mar. 2013). After selecting a plant comprising a transgene, plants comprising (at least) one transgenes can be crossed to form an F1 that comprises both transgenes. In progeny from these F1 (F2 or BC1) 1/500 progeny would have the two different transgenes recombined onto the same chromosome. The complex locus can then be bred as single genetic locus with both transgene traits. This process can be repeated to stack as many traits as desired.

Further uses for guide RNA/Cas endonuclease systems have been described (See for example: US20150082478 published 19 Mar. 2015, WO2015026886 published 26 Feb. 2015, US20150059010 published 26 Feb. 2015, WO2016007347 published 14 Jan. 2016, and PCT application WO2016025131 published 18 Feb. 2016) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Resulting characteristics from the gene editing compositions and methods described herein may be evaluated. Chromosomal intervals that correlate with a phenotype or trait of interest can be identified. A variety of methods well known in the art are available for identifying chromosomal intervals. The boundaries of such chromosomal intervals are drawn to encompass markers that will be linked to the gene controlling the trait of interest. In other words, the chromosomal interval is drawn such that any marker that lies within that interval (including the terminal markers that define the boundaries of the interval) can be used as a marker for a particular trait. In one embodiment, the chromosomal interval comprises at least one QTL, and furthermore, may indeed comprise more than one QTL. Close proximity of multiple QTLs in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identifies the same QTL or two different QTL. The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window.

In addition to the double-strand break inducing agents, site-specific base conversions can also be achieved to engineer one or more nucleotide changes to create one or more edits into the genome. These include for example, a site-specific base edit mediated by an C⋅G to T⋅A or an A⋅T to G⋅C base editing deaminase enzymes (Gaudelli et al., Programmable base editing of A⋅T to G⋅C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016):420-4. A catalytically “dead” or inactive Cas9 (dCas9), for example a catalytically inactive “dead” version of a Cas9 ortholog disclosed herein, fused to a cytidine deaminase or an adenine deaminase protein becomes a specific base editor that can alter DNA bases without inducing a DNA break. Base editors convert C→T (or G→A on the opposite strand) or an adenine base editor that would convert adenine to inosine, resulting in an A→G change within an editing window specified by the gRNA

Introduction of CRISPR-Cas System Components into a Cell

The methods and compositions described herein do not depend on a particular method for providing a composition to a cell, or for introducing a sequence into an organism or cell, only that the polynucleotide or polypeptide gains access to the interior of at least one cell of the organism. Introducing includes reference to the incorporation of a nucleic acid into a eukaryotic or prokaryotic cell where the nucleic acid may be incorporated into the genome of the cell, and includes reference to the transient (direct) provision of a nucleic acid, protein or polynucleotide-protein complex to the cell.

Methods for introducing polynucleotides or polypeptides or a polynucleotide-protein complex into cells or organisms are known in the art including, but not limited to, microinjection, electroporation, stable transformation methods, transient transformation methods, ballistic particle acceleration (particle bombardment), whiskers mediated transformation, Agrobacterium-mediated transformation, direct gene transfer, viral-mediated introduction, transfection, transduction, cell-penetrating peptides, mesoporous silica nanoparticle (MSN)-mediated direct protein delivery, topical applications, sexual crossing , sexual breeding, and any combination thereof.

For example, the guide polynucleotide (guide RNA, crNucleotide+tracrNucleotide, guide DNA and/or guide RNA-DNA molecule) can be introduced into a cell directly (transiently) as a single stranded or double stranded polynucleotide molecule. The guide RNA (or crRNA+tracrRNA) can also be introduced into a cell indirectly by introducing a recombinant DNA molecule comprising a heterologous nucleic acid fragment encoding the guide RNA (or crRNA+tracrRNA), operably linked to a specific promoter that is capable of transcribing the guide RNA (crRNA+tracrRNA molecules) in said cell. The specific promoter can be, but is not limited to, a RNA polymerase III promoter, which allow for transcription of RNA with precisely defined, unmodified, 5′- and 3′-ends (Ma et al., 2014, Mol. Ther. Nucleic Acids 3:e161; DiCarlo et al., 2013, Nucleic Acids Res. 41: 4336-4343; WO2015026887, published 26 Feb. 2015). Any promoter capable of transcribing the guide RNA in a cell can be used and includes a heat shock /heat inducible promoter operably linked to a nucleotide sequence encoding the guide RNA.

The Cas endonuclease, such as the Cas endonuclease described herein, can be introduced into a cell by directly introducing the Cas polypeptide itself (referred to as direct delivery of Cas endonuclease), the mRNA encoding the Cas protein, and/ or the guide polynucleotide/Cas endonuclease complex itself, using any method known in the art. The Cas endonuclease can also be introduced into a cell indirectly by introducing a recombinant DNA molecule that encodes the Cas endonuclease. The endonuclease can be introduced into a cell transiently or can be incorporated into the genome of the host cell using any method known in the art. Uptake of the endonuclease and/or the guided polynucleotide into the cell can be facilitated with a Cell Penetrating Peptide (CPP) as described in WO2016073433 published 12 May 2016. Any promoter capable of expressing the Cas endonuclease in a cell can be used and includes a heat shock /heat inducible promoter operably linked to a nucleotide sequence encoding the Cas endonuclease.

Direct delivery of a polynucleotide modification template into plant cells can be achieved through particle mediated delivery, and any other direct method of delivery, such as but not limiting to, polyethylene glycol (PEG)-mediated transfection to protoplasts, whiskers mediated transformation, electroporation, particle bombardment, cell-penetrating peptides, or mesoporous silica nanoparticle (MSN)-mediated direct protein delivery can be successfully used for delivering a polynucleotide modification template in eukaryotic cells, such as plant cells.

The donor DNA can be introduced by any means known in the art. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas endonuclease and the target site, the donor DNA is inserted into the transformed plant's genome.

Direct delivery of any of the guided Cas system components can be accompanied by direct delivery (co-delivery) of other mRNAs that can promote the enrichment and/or visualization of cells receiving the guide polynucleotide/Cas endonuclease complex components. For example, direct co-delivery of the guide polynucleotide/Cas endonuclease components (and/or guide polynucleotide/Cas endonuclease complex itself) together with mRNA encoding phenotypic markers (such as but not limiting to transcriptional activators such as CRC (Bruce et al. 2000 The Plant Cell 12:65-79) can enable the selection and enrichment of cells without the use of an exogenous selectable marker by restoring function to a non-functional gene product as described in WO2017070032 published 27 Apr. 2017.

Introducing a guide RNA/Cas endonuclease complex described herein, (representing the cleavage ready cascade described herein) into a cell includes introducing the individual components of said complex either separately or combined into the cell, and either directly (direct delivery as RNA for the guide and protein for the Cas endonuclease and cascade protein subunits, or functional fragments thereof) or via recombination constructs expressing the components (guide RNA, Cas endonuclease, cascade protein subunits, or functional fragments thereof). Introducing a guide RNA/Cas endonuclease complex into a cell includes introducing the guide RNA/Cas endonuclease complex as a ribonucleotide-protein into the cell. The ribonucleotide-protein can be assembled prior to being introduced into the cell as described herein. The components comprising the guide RNA/Cas endonuclease ribonucleotide protein (at least one Cas endonuclease, at least one guide RNA, at least one cascade protein subunits) can be assembled in vitro or assembled by any means known in the art prior to being introduced into a cell (targeted for genome modification as described herein).

Plant cells differ from human and animal cells in that plant cells comprise a plant cell wall which may act as a barrier to the direct delivery of the RGEN ribonucleoproteins and/or of the direct delivery of the RGEN components.

Direct delivery of the ribonucleoproteins, representing the cleavage ready cascade described herein, into plant cells can be achieved through particle mediated delivery (particle bombardment. Based on the experiments described herein, a skilled artesian can now envision that any other direct method of delivery, such as but not limiting to, polyethylene glycol (PEG)-mediated transfection to protoplasts, electroporation, cell-penetrating peptides, or mesoporous silica nanoparticle (MSN)-mediated direct protein delivery, can be successfully used for delivering RGEN ribonucleoproteins into plant cells.

Direct delivery of the ribonucleoprotein, allows for genome editing at a target site in the genome of a cell which can be followed by rapid degradation of the complex, and only a transient presence of the complex in the cell. This transient presence of the RGEN complex may lead to reduced off-target effects. In contrast, delivery of RGEN components (guide RNA, endonuclease) via plasmid DNA sequences can result in constant expression of RGENs from these plasmids which can intensify off target effects (Cradick, T. J. et al. (2013) Nucleic Acids Res 41:9584-9592; Fu, Y et al. (2014) Nat. Biotechnol. 31:822-826).

Direct delivery can be achieved by combining any one component of the guide RNA/Cas endonuclease complex (RGEN), representing the cleavage ready cascade described herein, (such as at least one guide RNA, at least one Cas protein, and at least one cascade protein), with a particle delivery matrix comprising a microparticle (such as but not limited to of a gold particle, tungsten particle, and silicon carbide whisker particle) (see also WO2017070032 published 27 Apr. 2017).

In one aspect the guide polynucleotide/Cas endonuclease complex, is a complex wherein the guide RNA and Cas endonuclease protein forming the guide RNA/Cas endonuclease complex are introduced into the cell as RNA and protein, respectively.

In one aspect the guide polynucleotide/Cas endonuclease complex, is a complex wherein the guide RNA and Cas endonuclease protein and the at least one protein subunit of a cascade forming the guide RNA/Cas endonuclease complex are introduced into the cell as RNA and proteins, respectively.

In one aspect the guide polynucleotide/Cas endonuclease complex, is a complex wherein the guide RNA and Cas endonuclease protein and the at least one protein subunit of a cascade forming the guide RNA/Cas endonuclease complex (cleavage ready cascade) are preassembled in vitro and introduced into the cell as a ribonucleotide-protein complex.

Protocols for introducing polynucleotides, polypeptides or polynucleotide-protein complexes into eukaryotic cells, such as plants or plant cells are generally known.

Alternatively, polynucleotides may be introduced into plant or plant cells by contacting cells or organisms with a virus or viral nucleic acids. Generally, such methods involve incorporating a polynucleotide within a viral DNA or RNA molecule. In some examples a polypeptide of interest may be initially synthesized as part of a viral polyprotein, which is later processed by proteolysis in vivo or in vitro to produce the desired recombinant protein. Methods for introducing polynucleotides into plants and expressing a protein encoded therein, involving viral DNA or RNA molecules, are known, see, for example, U.S. Pat. Nos. 5,889,191, 5,889,190, 5,866,785, 5,589,367 and 5,316,931.

### Cells and Plants

The presently disclosed polynucleotides and polypeptides can be introduced into a cell. Cells include, but are not limited to, human, non-human, animal, mammalian, bacterial, fungal, insect, yeast, non-conventional yeast, and plant cells as well as plants and seeds produced by the methods described herein. Any plant can be used with the compositions and methods described herein, including monocot and dicot plants, and plant elements.

Examples of monocot plants that can be used include, but are not limited to, corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

Examples of dicot plants that can be used include, but are not limited to, soybean (Glycine max), Brassica species (for example but not limited to: oilseed rape or Canola) (Brassica napus, B. campestris, Brassica rapa, Brassica. juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum).

Additional plants that can be used include safflower (Carthamus tinctorius), sweet potato (Ipomoea batatus), cassava (Manihot esculenta), coffee (Coffea spp.), coconut (Cocos nucifera), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), vegetables, ornamentals, and conifers.

Vegetables that can be used include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be used include pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis).

In certain embodiments of the disclosure, a fertile plant is a plant that produces viable male and female gametes and is self-fertile. Such a self-fertile plant can produce a progeny plant without the contribution from any other plant of a gamete and the genetic material comprised therein. Other embodiments of the disclosure can involve the use of a plant that is not self-fertile because the plant does not produce male gametes, or female gametes, or both, that are viable or otherwise capable of fertilization.

The present disclosure finds use in the breeding of plants comprising one or more introduced traits, or edited genomes.

A non-limiting example of how two traits can be stacked into the genome at a genetic distance of, for example, 5 cM from each other is described as follows: A first plant comprising a first transgenic target site integrated into a first DSB target site within the genomic window and not having the first genomic locus of interest is crossed to a second transgenic plant, comprising a genomic locus of interest at a different genomic insertion site within the genomic window and the second plant does not comprise the first transgenic target site. About 5% of the plant progeny from this cross will have both the first transgenic target site integrated into a first DSB target site and the first genomic locus of interest integrated at different genomic insertion sites within the genomic window. Progeny plants having both sites in the defined genomic window can be further crossed with a third transgenic plant comprising a second transgenic target site integrated into a second DSB target site and/or a second genomic locus of interest within the defined genomic window and lacking the first transgenic target site and the first genomic locus of interest. Progeny are then selected having the first transgenic target site, the first genomic locus of interest and the second genomic locus of interest integrated at different genomic insertion sites within the genomic window. Such methods can be used to produce a transgenic plant comprising a complex trait locus having at least 1, 2, 3, 4, 5, 6, 7, 8, 9, 10, 11, 12, 13, 14,15, 16, 17, 19, 19, 20, 21, 22, 23, 24, 25, 26, 27, 28, 29, 30, 31 or more transgenic target sites integrated into DSB target sites and/or genomic loci of interest integrated at different sites within the genomic window. In such a manner, various complex trait loci can be generated.

While the invention has been particularly shown and described with reference to a preferred embodiment and various alternate embodiments, it will be understood by persons skilled in the relevant art that various changes in form and details can be made therein without departing from the spirit and scope of the invention. For instance, while the particular examples below may illustrate the methods and embodiments described herein using a specific plant, the principles in these examples may be applied to any plant. Therefore, it will be appreciated that the scope of this invention is encompassed by the embodiments of the inventions recited herein and in the specification rather than the specific examples that are exemplified below. All cited patents, applications, and publications referred to in this application are herein incorporated by reference in their entirety, for all purposes, to the same extent as if each were individually and specifically incorporated by reference.

### EXAMPLES

In the following Examples, unless otherwise stated, parts and percentages are by weight and degrees are Celsius. It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims

### Example 1: Identification of Type I-E CRISPR-Cas Components for Eukaryotic Genome Editing

To establish the capability of Type I-E CRISPR (clustered regularly interspaced palindromic repeats)-Cas (CRISPR associated) systems for genome editing in eukaryotes, as exemplified in maize, the genes involved in CRISPR RNA (crRNA) maturation (cas6 (cse3 or casE)), DNA target recognition (cas5 (cas5e or casD), cas7 (cse4 or casC), cse1 (casA, cas8, or cas8e) and cse2 (casB or small subunit) and DNA target cleavage (cas3) (Makarova, K. S. et al., 2011, Nature Rev. Microbiol. 9:467-477 and Makarova, K. S. et al., 2015, Nature Rev. Microbiol. 13:722-736) from the Type I-E CRISPR-Cas system from Streptococcus thermophilus DGCC7710 (Sth7710) were identified. This was accomplished by first retrieving the genomic sequence for Sth7710 (NCBI accession number: NZ_AWVZ00000000.1) and examining it for the presence of CRISPR repeats using minCED (Bland, C. et al. (2007) BMC Bioinformatics, 8:209). In total, 4 arrays of CRISPRs each with a different repeat sequence were identified. Next, genomic sequence regions adjacent to the identified CRISPR arrays (about 10 kb 5 prime and 3 prime) were examined for the presence of open-reading frames (ORFs) encoding proteins with homology to Cas3, the signature protein of Type I CRISPR-Cas systems, by comparisons with NCBI protein databases using the PSI-BLAST program (Altschul, S. F. et al., 1997, Nucleic Acids Res. 25:3389-3402). One of the CRISPR arrays comprising a 28 bp repeat with the sequence consensus of 5′-GTTTTTCCCGCACACGCGGGGGTGATCC-3′ (SEQ ID NO:1) was demonstrated to be adjacent to an ORF encoding a Cas3 protein. Next, genes encoding the other proteins typical of a Type I-E CRISPR-Cas system (Cas5, Cas6, Cas7, Cse1, Cse2, Cas1 and Cas2) (Makarova, K. S. et al., 2015, Nature Rev. Microbiol. 13:722-736) were also identified within the locus (SEQ ID NOs: 2-9). This was accomplished by first translating the ORFs between the cas3 gene and CRISPR array into proteins using ORF Finder (Stothard, P. (2000) Biotechniques 28:1102-1104) followed by comparisons with NCBI protein databases using the PSI-BLAST program (Altschul, S. F. et al., 1997). As shown in FIG. 1, the resulting genes produced an operon structure typical of a Type I-E CRISPR-Cas system (Makarova, K. S. et al., 2015, Nature Rev. Microbiol. 13:722-736). The genes and encoded proteins responsible for CRISPR RNA maturation (cas6), RNA directed DNA target recognition (cas5, cas7, cse1 and cse2) and DNA target cleavage (cas3) are listed in Table 1. The cast gene (SEQ ID NO:8) encoded the Casl protein (SEQ ID NO:16). The cast gene (SEQ ID NO:9) encoded the Cas2 protein (SEQ ID NO:17).

TABLE 1A
Sequence of Type I-E CRISPR-Cas genes and proteins
from Streptococcus thermophilus DGCC7710 responsible
for CRISPR RNA maturation, DNA target recognition
and cleavage (SEQ ID NOs: 2-15)

| Gene | Polynucleotide Sequence | Polypeptide Sequence |
|---|---|---|
| Name | (SEQ ID NO) | (SEQ ID NO) |
| cas5 | 2 | 10 |
| cas6 | 3 | 11 |
| cas7 | 4 | 12 |
| cse1 | 5 | 13 |
| cse2 | 6 | 14 |
| cas3 | 7 | 15 |

The methods described above were also used to identify the genes involved in CRISPR RNA (crRNA) maturation (cas6), DNA target recognition (cas5, cas7, cse1 and cse2) and DNA target cleavage (cas3) from other Type I-E CRISPR-Cas systems including but not limited to Streptococcus mutans (Smu), Peptostreptococcus anaerobius (Pan), Gardnerella vaginalis (Gva), Pseudomonas 1sp. S-6-2 (Psp), and Ralstonia solanacearum (Rso) (Table 1B).

TABLE 1B
Sequences of additional Type I-E CRISPR-Cas genes and proteins
from Streptococcus mutans (Smu), Peptostreptococcus anaerobius
(Pan), Gardnerella vaginalis (Gva), Pseudomonas sp.
S-6-2 (Psp), and Ralstonia solanacearum (Rso) responsible
for CRISPR RNA maturation, DNA target recognition and
cleavage (SEQ ID NOs: 237-296)

| Gene | Polynucleotide Sequence | Polypeptide Sequence |
|---|---|---|
| Name | (SEQ ID NO) | (SEQ ID NO) |
| Smu cas5 | 237 | 267 |
| Smu cas6 | 238 | 268 |
| Smu cas7 | 239 | 269 |
| Smu cse1 | 240 | 270 |
| Smu cse2 | 241 | 271 |
| Smu cas3 | 242 | 272 |
| Pan cas5 | 243 | 273 |
| Pan cas6 | 244 | 274 |
| Pan cas7 | 245 | 275 |
| Pan cse1 | 246 | 276 |
| Pan cse2 | 247 | 277 |
| Pan cas3 | 248 | 278 |
| Gva cas5 | 249 | 279 |
| Gva cas6 | 250 | 280 |
| Gva cas7 | 251 | 281 |
| Gva cse1 | 252 | 282 |
| Gva cse2 | 253 | 283 |
| Gva cas3 | 254 | 284 |
| Psp cas5 | 255 | 285 |
| Psp cas6 | 256 | 286 |
| Psp cas7 | 257 | 287 |
| Psp cse1 | 258 | 288 |
| Psp cse2 | 259 | 289 |
| Psp cas3 | 260 | 290 |
| Rso cas5 | 261 | 291 |
| Rso cas6 | 262 | 292 |
| Rso cas7 | 263 | 293 |
| Rso cse1 | 264 | 294 |
| Rso cse2 | 265 | 295 |
| Rso cas3 | 266 | 296 |

### Example 2: Optimization of Type I-E CRISPR-Cas Genes for Maize Genome Editing

To confer efficient expression in maize cells, the Type I-E CRISPR-Cas genes from Streptococcus thermophilus DGCC7710 (Sth7710) responsible for CRISPR RNA (crRNA) maturation (cash) and RNA guided DNA target recognition (cas5, cas7, cse1 and cse2) Makarova, K. S. et al., 2015, Nature Rev. Microbiol. 13:722-736), termed cascade (CRISPR-associated complex for antiviral defense) (Brouns, S. J. et al. (2008) Science. 321:960-964), were. Briefly, genes were codon optimized using Zea mays codon tables, conditioned for ideal GC content, and repetitive sequences and gene destabilizing features such as MITE and DST sites removed where possible. Since the Cas5, Cas6, Cas7, Cse1 and Cse2 Type I-E proteins form a multi-subunit protein complex (Brouns, S. J. et al. (2008) Science. 321:960-964, Sinkunas, T. et al. (2013) EMBO J. 32:385-394 and Makarova, K. S. et al., 2015, Nature Rev. Microbiol. 13:722-736), not all of the proteins encoded within these genes may need a nuclear localization signal (NLS) for localization to occur within the nucleus of a eukaryotic cell. Therefore, the maize codon optimized Sth7710 Type I-E cascade genes were engineered with and without a sequence encoding the NLS from the Simian virus 40 (SV40) (PKKKRKV, SEQ ID NO:18) as described in Table 2 for testing.

TABLE 2
sequences and gene products resulting from the combination
of the SV40 encoding nuclear localization sequence and Type
I-E genes cas5, cas6, cas7, cse1 and cse2 from Streptococcus thermophihis
DGCC7710 (SEQ ID NOs: 10-14, 19-35)

| | Polynucleotide Sequence | Polypeptide Sequence |
|---|---|---|
| Gene Name | (SEQ ID NO) | (SEQ ID NO) |
| cas5 no NLS | 19 | 10 |
| cas6 no NLS | 20 | 11 |
| cas7 no NLS | 21 | 12 |
| cse1 no NLS | 22 | 13 |
| cse2 no NLS | 23 | 14 |
| cas5 N terminal NLS | 24 | 30 |
| cas5 C terminal NLS | 25 | 31 |
| cse1 N terminal NLS | 26 | 32 |
| cas6 C terminal NLS | 27 | 33 |
| cas7 C terminal NLS | 28 | 34 |
| cse2 C terminal NLS | 29 | 35 |

The nucleotide sequences encoding the genes were then operably linked to a maize ubiquitin (UBI) promoter (SEQ ID NO:36), maize UBI 5 prime untranslated region (UTR) (SEQ ID NO:37), maize UBI intron 1 (SEQ ID NO:38) and suitable terminator by standard molecular biological techniques. The key components of the resulting Type I-E expression cassettes are illustrated in FIG. 2 and are listed in Table 3.

TABLE 3
Type I-E cascade gene expression cassettes for RNA
directed DNA target recognition from Streptococcus thermophilus
DGCC7710 (SEQ ID NOs: 39-49)

| Gene Expression Cassette Name | SEQ ID NO: |
|---|---|
| UBI cas5 no NLS | 39 |
| UBI cas6 no NLS | 40 |
| UBI cas7 no NLS | 41 |
| UBI cse1 no NLS | 42 |
| UBI cse2 no NLS | 43 |
| UBI cas5 N terminal NLS | 44 |
| UBI cas5 C terminal NLS | 45 |
| UBI cse1 N terminal NLS | 46 |
| UBI cas6 C terminal NLS | 47 |
| UBI cas7 C terminal NLS | 48 |
| UBI cse2 C terminal NLS | 49 |

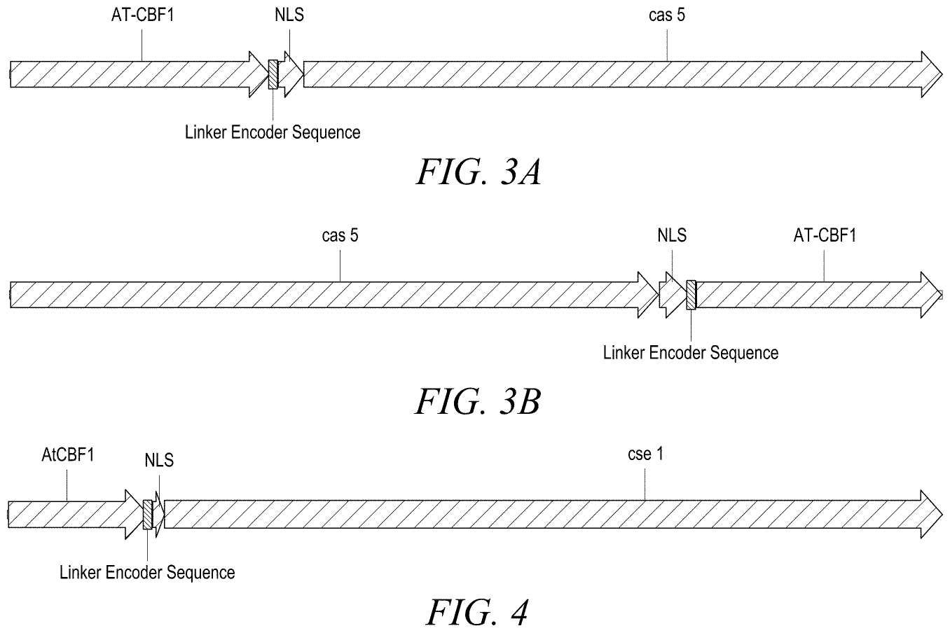

To monitor Type I-E cascade complex formation and RNA guided target DNA binding in maize cells, a sequence encoding only the acidic transcriptional activator motif from the Arabidopsis CBF1 protein (AtCBF1) (SEQ ID NO:50) (Gilmour, S. J. et al., 1998, Plant J. 16:433-442) was maize codon optimized and linked to the cas5 and cse1 cascade genes. To establish the optimal linkage of the AtCBF1 activator motif to the cas5 and cse1 genes, different sequences encoding protein linkers, GRA or 30XQ, were used to join cas5 and cse1 genes with the sequence encoding the AtCBF1 activation motif (see FIGS. 3A, 3 B and 4). For the cas5 gene, the AtCBF1 transcriptional activator motif was linked to both the 5 and 3 prime ends of the gene versions comprising NLSs (SEQ ID NOs: 24 and 25) (see FIGS. 3A and B) resulting in SEQ ID NOs: 47-50. For the cse1 gene, the AtCBF1 transcriptional activator motif was only linked to the 5 prime end of the gene version comprising a NLS (SEQ ID NO:26) (see FIG. 4) resulting in SEQ ID NOs: 55 and 56. The key components of the resulting AtCBF1 transcriptional activator fused cas5 and cse1 genes are listed in Table 4.

TABLE 4
Key components comprising the AtCBF1 transcriptional activator linked cas5
and cse1 genes from Streptococcus thermophilus DGCC7710 (SEQ ID NOs: 51-62),
If a particular sequence is not applicable, it is indicated with a N/A,

| | Coordinates | | | Coordinates of | |
|---|---|---|---|---|---|
| AtCBF1 linked | of AtCBF1 | Coordinates of | Coordinates of | cas5 or cse1 | Gene |
| cas5 or cse1 | transcriptional | sequence | sequence | gene | Product |
| gene | activator | encoding 30XQ | encoding GRA | comprising a | (SEQ ID |
| (SEQ ID NO) | motif | linker | linker | NLS | NO) |
| 51 (cas5) | 1-297 | 298-402 | N/A | 403-1164 | 57 |
| 52 (cas5) | 1-297 | N/A | 298-306 | 307-1068 | 58 |
| 53 (cas5) | 871-1164 | 766-870 | N/A | 1-765 | 59 |
| 54 (cas5) | 775-1071 | N/A | 766-774 | 7-765 | 60 |
| 55 (cse1) | 1-297 | 298-402 | N/A | 403-2097 | 61 |
| 56 (cse1) | 1-297 | N/A | 298-306 | 307-2001 | 62 |

For use as a positive control in targeted DNA binding and gene activation in maize cells, the Streptococcus pyogenes (Spy) cas9 gene comprising N- and C-terminal NLSs and the ST-LS1 Intron 2, for example described in US patent application WO2015026883A1 and Svitashev et al. (2015) Plant Physiology). 169:931-945 (SEQ ID NO:63) was modified as described in Jinek, M. et al., 2012, Science. 337:816-821 to generate a cleavage inactive or deactivated Spy Cas9 (Spy dCas9). These modifications introduced a pair of alanine substitutions at positions 19 and 849 of the expressed protein (comprising C- and N-terminal NLSs) (SEQ ID NO:64). Next, the sequence encoding the transcriptional activator motif from AtCBF1 (SEQ ID NO:50) was linked to the 3 prime end of the Spy dcas9 gene using two different sequences encoding protein linkers, 30XQ or GRA, and maize codon optimized in the context of the Spy dcas9 gene resulting in genes SEQ ID NO:65 and SEQ ID NO:66. The key components of the resulting AtCBF1 transcriptional activator fused Spy dcas9 genes are listed in Table 5.

TABLE 5
Key components comprising the AtCBF1 transcriptional activator linked Spy dcas9 genes (SEQ
ID NOs: 65-68), If a particular sequence is not applicable, it is indicated with a N/A.

| | Coordinates of | | | | |
|---|---|---|---|---|---|
| | Spy dcas9 | | | | |
| | gene | Coordinates of | Coordinates of | Coordinates of | Gene |
| AtCBF1 linked | comprising | sequence | sequence | AtCBF1 | Product |
| Spy dcas9 gene | NLSs and ST- | encoding 30XQ | encoding GRA | transcriptional | (SEQ ID |
| (SEQ ID NO) | LS1 Intron 2 | linker | linker | activator motif | NO) |
| 65 | 1-4374 | 4375-4479 | N/A | 4480-4776 | 67 |
| 66 | 1-4374 | N/A | 4375-4383 | 4384-4680 | 68 |

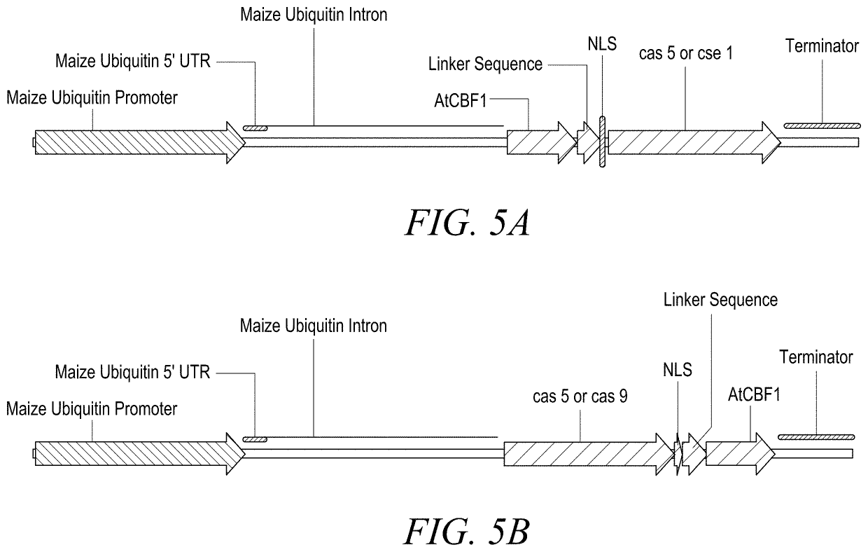

Next, the AtCBF1 transcriptional activator fused genes (cas5, cse1 and Spy dcas9) were then operably linked to a maize UBI promoter (SEQ ID NO:36), maize UBI 5 prime UTR (SEQ ID NO:37), maize UBI intron 1 (SEQ ID NO:38) and suitable terminator by standard molecular biological techniques. The key components of the resulting AtCBF1 transcriptional activator linked gene expression cassettes are illustrated in FIG. 5A and B and the respective sequences are listed in Table 6.

TABLE 6
expression cassettes for cas5, cse1 and Spy dcas9 genes linked to
the AtCBF1 transcriptional activation motif (SEQ ID NOs: 69-76)

| Gene Expression | SEQ |
|---|---|
| Cassette Name | ID NO: |
| UBI cas5 gene with N-terminal | 69 |
| NLS linked by 30XQ to AtCBF1 motif | |
| UBI cas5 gene with N-terminal | 70 |
| NLS linked by GRA to AtCBF1 motif | |
| UBI cas5 gene with C-terminal | 71 |
| NLS linked by 30XQ to AtCBF1 motif | |
| UBI cas5 gene with C-terminal | 72 |
| NLS linked by GRA to AtCBF1 motif | |
| UBI cse1 gene with N-terminal | 73 |
| NLS linked by 30XQ to AtCBF1 motif | |
| UBI cse1 gene with N-terminal | 74 |
| NLS linked by GRA to AtCBF1 motif | |
| UBI Spy dcas9 gene linked | 75 |
| with AtCBF1 transcriptional activator by 30XQ | |
| UBI Spy dcas9 gene linked with | 76 |
| AtCBF1 transcriptional activator by GRA | |

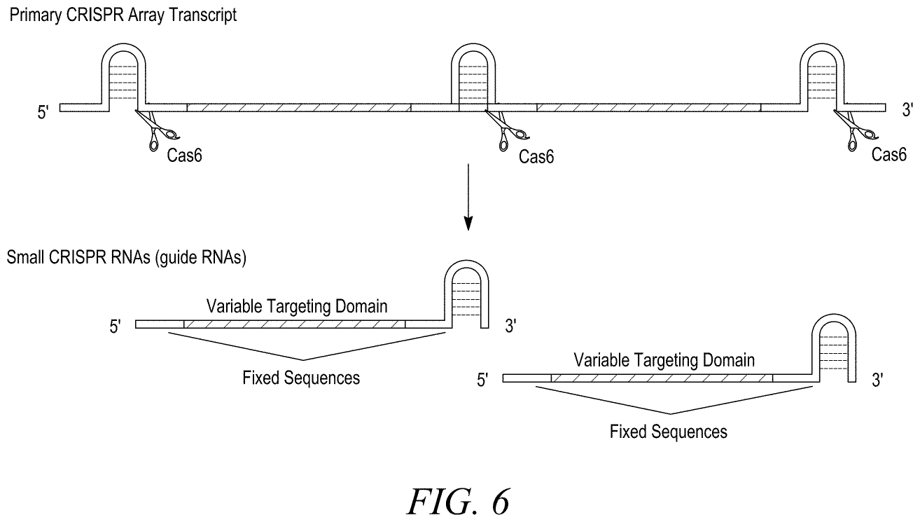

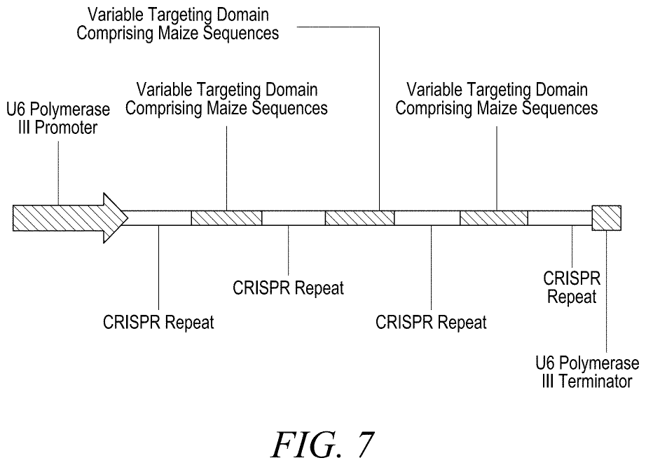

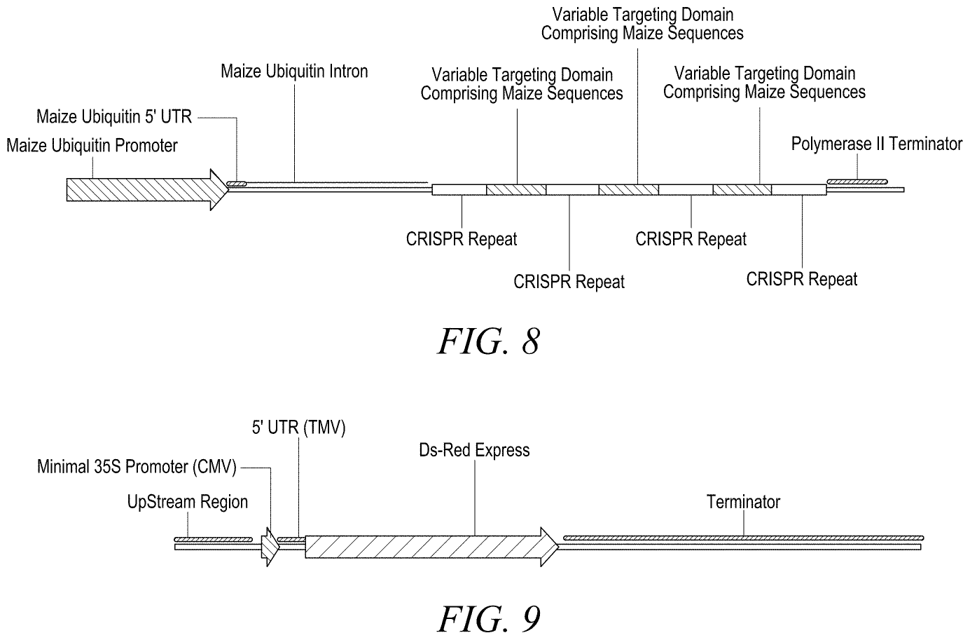

The multi-protein complex comprised of Cas5, Cas6, Cas7, Cse1 and Cse2 Type I-E Sth7710 proteins, termed cascade, is directed by small CRISPR RNAs (referred to herein as guide RNAs) to bind DNA target sites (Jore, M. M. et al. (2011) Nat. Struct. Mol. Biol. 18:529-536 and Sinkunas, T. et al. (2013) EMBO J. 32:385-394). These guide RNAs are comprised of a ˜33 nt sequence that serves to direct cascade DNA target recognition by base pairing with one strand of the DNA target site (cascade variable targeting domain) that is adjacent to an appropriate protospacer adjacent motif (PAM) (Jore, M. M. et al. (2011) Nat. Struct. Mol. Biol. 18:529-536 and Sinkunas, T. et al. (2013) EMBO J. 32:385-394). Flanking the ˜33 nt variable targeting domain are fixed sequences comprised of a ˜7 nt 5 prime sequence and a ˜21 nt 3 prime hairpin comprising sequence (FIG. 6). These fixed flanking sequences are the result of cleavage within the repeat sequences of the primary CRISPR array transcript by the Cas6 (Cse3) protein in Type I-E CRISPR-Cas systems (FIG. 6) (Gesner, E. M. et al. (2011) Nat. Struct. Mol. Biol. 18:688-692). To transcribe small RNAs necessary for directing cascade complex DNA target recognition and binding in maize cells, a U6 polymerase III promoter (SEQ ID NO:77) and terminator (TTTTTTTT) were isolated from maize and operably fused to the ends of DNA sequences mimicking a Type I-E CRISPR array as shown in FIG. 7 so that upon transcription the Cas6 protein may recognize the CRISPR array transcript and process it into separate guide RNAs. An important difference distinguishing a Type I-E CRISPR array from the array was that the cascade variable targeting domain was now altered to comprise DNA sequences (adjacent to an appropriate PAM) that may be found in maize cells. Additionally, a construct was assembled to test the ability of Type I-E guide RNAs to be transcribed from a polymerase II promoter. This plasmid DNA transcriptional cassette comprised the maize UBI promoter (SEQ ID NO:36), maize UBI 5 prime UTR (SEQ ID NO:37), maize UBI intron 1 (SEQ ID NO:38) and CRISPR array followed by a suitable polymerase II terminator (FIG. 8). The Type I-E guide RNA transcribed from these cassettes (after ribonuclease cleavage by Cas6) is as listed in SEQ ID NO:78 (Ns represent the variable targeting domain where any nucleotide may be present). To target the GACT expression construct described in Example 4 for gene activation in maize cells, three 33 nt sequences 3 prime of an appropriate PAM for Sth7710 Type I-E cascade (A or AA) (Sinkunas, T. et al. (2013) EMBO 1 32:385-394) capable of binding to 4 target sites upstream of the 35S CAMV minimal promoter were captured and placed into an array (SEQ ID NO:79). Next, the GACT targeting array was synthesized (GeneScript) and cloned into the polymerase II and III guide RNA expression constructs described herein resulting in SEQ ID NOs: 80 and 81, respectively.

To enable DNA target cleavage following DNA target recognition, the gene encoding the Type I-E endonuclease, cas3, from Sth7710 (SEQ ID NO:7) was maize codon optimized (SEQ ID NO:82). In order to eliminate its expression in coli and Agrobacterium, the potato ST-LS1 intron 2 (SEQ ID NO:83) was introduced resulting in SEQ ID NO:84. The gene product of the Sth7710 cas3 gene was also converted into a nickase by altering the codon at positions 1543-1545 to encode an alanine instead of an aspartic acid resulting in SEQ ID NO:85. This change has been previously reported to abolish the helicase activity of Sth7710 Cas3 resulting in a Cas3 variant which nicks one strand of the double-stranded DNA target site recognized by the cascade complex (Sinkunas, T. et al. (2013) EMBO J. 32:385-394). To facilitate nuclear localization of the Cas3 endonuclease protein and its variant(s) in maize cells, a nucleotide sequence encoding the Simian virus 40 (SV40) monopartite nuclear localization signal (PKKKRKV, SEQ ID NO:18) was included on either the 5 prime end, 3 prime end or both 5 and 3 prime ends of the cas3 genes described above resulting in the sequences and gene products listed in Table 7.

TABLE 7
Gene sequences and products for the cas3 gene
and variants from Streptococcus thermophilus
DGCC7710 (Sth7710) (SEQ ID NOs: 86-97)

| | Gene | Gene |
|---|---|---|
| | Sequence | Product |
| | (SEQ | (SEQ |
| Gene Name | ID NO:) | ID NO:) |
| cas3 with N-terminal NLS | 86 | 92 |
| cas3 with C-terminal NLS | 87 | 93 |
| cas3 with both N- and C-terminal NLSs | 88 | 94 |
| cas3 nickase with N-terminal NLS | 89 | 95 |
| cas3 nickase with C-terminal NLS | 90 | 96 |
| cas3 nickase with both | 91 | 97 |
| N- and C-terminal NLSs | | |

The nucleotide sequences encoding the cas3 genes were then operably linked to a maize UBI promoter (SEQ ID NO:36), maize UBI 5 UTR (SEQ ID NO:37), maize UBI intron 1 (SEQ ID NO:38) and suitable terminator by standard molecular biological techniques. The sequences of the resulting Type I-E expression cassettes are listed in Table 8.

TABLE 8A
Type I-E cas3 gene expression cassettes (SEQ ID NOs: 98-103)

| Gene Expression Cassette Name | SEQ ID NO: |
|---|---|
| UBI cas3 with N-terminal NLS | 98 |
| UBI cas3 with C-terminal NLS | 99 |
| UBI cas3 with both N- and C-terminal NLSs | 100 |
| UBI cas3 nickase with N-terminal NLS | 101 |
| UBI cas3 nickase with C-terminal NLS | 102 |
| UBI cas3 nickase with both N- and C-terminal NLSs | 103 |

Another approach to enable DNA cleavage is to link a nuclease domain (for example but not limited to the nuclease domain of a Type II restriction endonuclease (i.e. FokI (Kim, Y. et al. (1996) Proc. Natl. Acad. Sci. USA. 93:1156-1160) or a nuclease domain from a homing endonuclease (i.e. I-TevI (Van Roey, P. et al. (2002) Nat. Struct. Biol. 9:806-811) to a subunit of the cascade complex. To accomplish this, a sequence encoding the amino terminal cleavage domain of the GIY-YIG homing endonuclease I-TevI (SEQ ID NO:165) was (SEQ ID NO:166) and appended to the 5 prime end of the cas6 (SEQ ID NO:20) and cse1 (SEQ ID NO:22) genes. To establish optimal linkage between the cascade subunit and the I-TevI nuclease domain (if needed), sequences encoding 3 different protein linker variants (GRA, (G₂S)₃, and XTEN) were also included between the sequences encoding the nuclease domain and the cascade gene. To ensure nuclear localization, sequences encoding the SV40 NLS (PKKKRKV, SEQ ID NO:18) were added to the 3 prime end of each gene. Then, to eliminate expression in E. coli and Agrobacterium, the potato ST-LS1 intron 2 (SEQ ID NO:83) was introduced into each gene variant resulting in the nuclease linked cascade genes listed in Table 8B.

TABLE 8B
Gene sequences and products for the nuclease enabled
cas6 and cse1 genes from Streptococcus thermophilus
DGCC7710 (Sth7710) (SEQ ID NOs: 167-182)

| | Gene | Gene |
|---|---|---|
| | Sequence | Product |
| | (SEQ | (SEQ |
| Gene Name | ID NO:) | ID NO:) |
| Nuclease enabled | 167 | 175 |
| I-Tevl:cas6:NLS | | |
| Nuclease enabled | 168 | 176 |
| I-Tevl:GRA:cas6:NLS | | |
| Nuclease enabled | 169 | 177 |
| I-Tevl:(G₂S)₃:cas6:NLS | | |
| Nuclease enabled | 170 | 178 |
| I-Tevl:XTEN:cas6:NLS | | |
| Nuclease enabled | 171 | 179 |
| I-Tevl:cse1:NLS | | |
| Nuclease enabled | 172 | 180 |
| I-Tevl:GRA:cse1:NLS | | |
| Nuclease enabled | 173 | 181 |
| I-Tevl:(G₂S)₃:cse1:NLS | | |
| Nuclease enabled | 174 | 182 |
| I-Tevl:XTEN:cse1:NLS | | |

The nucleotide sequences encoding the nuclease equipped cas6 (SEQ ID NOs: 167-170) and cse1 (SEQ ID NOs: 171-174) genes were then operably linked to a maize UBI promoter (SEQ ID NO:36), maize UBI 5 UTR (SEQ ID NO:37), maize UBI intron 1 (SEQ ID NO:38) and suitable terminator by standard molecular biological techniques. The sequences of the resulting nuclease enabled Type I-E expression cassettes are listed in Table 8C.

TABLE 8C
Type I-E nuclease enabled cas6 and cse1 gene
expression cassettes (SEQ ID NOs: 183-190)

| Gene Expression Cassette Name | SEQ ID NO: |
|---|---|
| UBI nuclease enabled I-Tevl:cas6:NLS | 183 |
| UBI nuclease enabled I-Tevl:GRA:cas6:NLS | 184 |
| UBI nuclease enabled I-Tevl:(G₂S)₃:cas6:NLS | 185 |
| UBI nuclease enabled I-Tevl:XTEN:cas6:NLS | 186 |
| UBI nuclease enabled I-Tevl:cse1:NLS | 187 |
| UBI nuclease enabled I-Tevl:GRA:cse1:NLS | 188 |
| UBI nuclease enabled I-Tevl:(G₂S)₃:cse1:NLS | 189 |
| UBI nuclease enabled I-Tevl:XTEN:cse1:NLS | 190 |

### Example 3: Transformation of Type I-E Cascade and Guide Polynucleotide(s) for Genome Manipulation

In some aspects, the compositions disclosed herein may be utilized to modify the transcriptional status of a gene or a target polynucleotide in the genome of a cell. In some aspects, said cell is a eukaryotic cell. In one example of a eukaryotic cell, a plant cell is used. Transformation of a eukaryotic cell with a Type I-E cascade and associated guide polynucleotide can be accomplished by various methods known to be effective in plants, including particle-mediated delivery, Agrobacterium-mediated transformation, PEG-mediated delivery, and electroporation. It is appreciated that any method known in the art may be utilized. Example methods are described below.

### Particle-Mediated Delivery

Transformation of maize immature embryos using particle delivery is performed as follows. Media recipes follow below.

The ears are husked and surface sterilized in 30% Clorox bleach plus 0.5% Micro detergent for 20 minutes, and rinsed two times with sterile water. The immature embryos are isolated and placed embryo axis side down (scutellum side up), 25 embryos per plate, on 560Y medium for 4 hours and then aligned within the 2.5-cm target zone in preparation for bombardment. Alternatively, isolated embryos are placed on 560L (Initiation medium) and placed in the dark at temperatures ranging from 26° C. to 37° C. for 8 to 24 hours prior to placing on 560Y for 4 hours at 26° C. prior to bombardment as described above.

Plasmids comprising the Type I cascade and associated guide polynucleotide and optionally donor DNA are constructed using standard molecular biology techniques and co-bombarded with plasmids containing the developmental genes ODP2 (AP2 domain transcription factor ODP2 (Ovule development protein 2); US20090328252 A1) and Wushel (US2011/0167516).

The plasmids and optionally donor DNA are precipitated onto 0.6 micrometer (average diameter) gold pellets using a water-soluble cationic lipid transfection reagent as follows. DNA solution is prepared on ice using 1 μg of plasmid DNA and optionally other constructs for co-bombardment such as 50 ng (0.5 μl) of each plasmid containing the developmental genes ODP2 (AP2 domain transcription factor ODP2 (Ovule development protein 2); US20090328252 A1) and Wuschel. To the pre-mixed DNA, 20 μl of prepared gold particles (15 mg/ml) and 5 μl of a water-soluble cationic lipid transfection reagent is added in water and mixed carefully. Gold particles are pelleted in a microfuge at 10,000 rpm for 1 min and supernatant is removed. The resulting pellet is carefully rinsed with 100 ml of 100% EtOH without resuspending the pellet and the EtOH rinse is carefully removed. 105 μl of 100% EtOH is added and the particles are resuspended by brief sonication. Then, 10 μl is spotted onto the center of each macrocarrier and allowed to dry about 2 minutes before bombardment.

Alternatively, the plasmids and DNA of interest are precipitated onto 1.1 μm (average diameter) tungsten pellets using a calcium chloride (CaCl2) precipitation procedure by mixing 100 μl prepared tungsten particles in water, 10 μl (1 μg) DNA in Tris EDTA buffer (1 total DNA), 100 μl 2.5 M CaCl2, and 10 μl 0.1 M spermidine. Each reagent is added sequentially to the tungsten particle suspension, with mixing. The final mixture is sonicated briefly and allowed to incubate under constant vortexing for 10 minutes. After the precipitation period, the tubes are centrifuged briefly, liquid is removed, and the particles are washed with 500 ml 100% ethanol, followed by a 30 second centrifugation. Again, the liquid is removed, and 105 μl of 100% ethanol is added to the final tungsten particle pellet. For particle gun bombardment, the tungsten/DNA particles are briefly sonicated. 10 μl of the tungsten/DNA particles is spotted onto the center of each macrocarrier, after which the spotted particles are allowed to dry about 2 minutes before bombardment.

The sample plates are bombarded at level #4 with a Biorad Helium Gun. All samples receive a single shot at 450 PSI, with a total of ten aliquots taken from each tube of prepared particles/DNA.

Following bombardment, the embryos are incubated on 560P (maintenance medium) for 12 to 48 hours at temperatures ranging from 26C to 37C, and then placed at 26C. After 5 to 7 days the embryos are transferred to 560R selection medium containing 3 mg/liter Bialaphos, and subcultured every 2 weeks at 26C. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to a lighted culture room. Approximately 7-10 days later, developing plantlets are transferred to 272V hormone-free medium in tubes for 7-10 days until plantlets are well established. Plants are then transferred to inserts in flats (equivalent to a 2.5″ pot) containing potting soil and grown for 1 week in a growth chamber, subsequently grown an additional 1-2 weeks in the greenhouse, then transferred to Classic 600 pots (1.6 gallon) and grown to maturity. Plants are monitored and scored for transformation efficiency, and/or modification of regenerative capabilities.

Initiation medium (560L) comprises 4.0 g/l N6 basal salts (SIGMA C-1416), 1.0 ml/l Eriksson's Vitamin Mix (1000X SIGMA-1511), 0.5 mg/1 thiamine HCl, 20.0 g/l sucrose, 1.0 mg/1 2,4-D, and 2.88 g/l L-proline (brought to volume with D-I H2O following adjustment to pH 5.8 with KOH); 2.0 g/l Gelrite (added after bringing to volume with D-I H2O); and 8.5 mg/l silver nitrate (added after sterilizing the medium and cooling to room temperature).

Maintenance medium (560P) comprises 4.0 g/l N6 basal salts (SIGMA C-1416), 1.0 ml/l Eriksson's Vitamin Mix (1000X SIGMA-1511), 0.5 mg/l thiamine HCl, 30.0 g/l sucrose, 2.0 mg/l 2,4-D, and 0.69 g/l L-proline (brought to volume with D-I H2O following adjustment to pH 5.8 with KOH); 3.0 g/l Gelrite (added after bringing to volume with D-I H2O); and 0.85 mg/l silver nitrate (added after sterilizing the medium and cooling to room temperature).

Bombardment medium (560Y) comprises 4.0 g/l N6 basal salts (SIGMA C-1416), 1.0 ml/l Eriksson's Vitamin Mix (1000X SIGMA-1511), 0.5 mg/l thiamine HCl, 120.0 g/l sucrose, 1.0 mg/l 2,4-D, and 2.88 g/l L-proline (brought to volume with D-I H2O following adjustment to pH 5.8 with KOH); 2.0 g/l Gelrite (added after bringing to volume with D-I H2O); and 8.5 mg/1 silver nitrate (added after sterilizing the medium and cooling to room temperature).

Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H2O following adjustment to pH 5.8 with KOH); 3.0 g/l Gelrite (added after bringing to volume with D-I H2O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H2O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H2O after adjusting to pH 5.6); 3.0 g/l Gelrite (added after bringing to volume with D-I H2O); and 1.0 mg/l indoleacetic acid and 3.0 mg/l bialaphos (added after sterilizing the medium and cooling to 60° C.).

Hormone-free medium (272V) comprises 4.3 g/l MS salts (GIBCO 11117-074), 5.0 m1/1 MS vitamins stock solution (0.100 g/l nicotinic acid, 0.02 g/l thiamine HCL, 0.10 g/l pyridoxine HCL, and 0.40 g/1 glycine brought to volume with polished D-I H2O), 0.1 g/l myo-inositol, and 40.0 g/l sucrose (brought to volume with polished D-I H2O after adjusting pH to 5.6); and 6 g/l bacto-agar (added after bringing to volume with polished D-I H2O), sterilized and cooled to 60° C.

Agrobacterium-Mediated Transformation

Agrobacterium-mediated transformation is performed essentially as described in Djukanovic et al. (2006) Plant Biotech J 4:345-57. Briefly, 10-12 day old immature embryos (0.8-2.5 mm in size) are dissected from sterilized kernels and placed into liquid medium (4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.690 g/L L-proline, 68.5 g/L sucrose, 36.0 g/L glucose, pH 5.2). After embryo collection, the medium is replaced with 1 ml Agrobacterium at a concentration of 0.35-0.45 OD550. Maize embryos are incubated with Agrobacterium for 5 min at room temperature, then the mixture is poured onto a media plate containing 4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.690 g/L L-proline, 30.0 g/L sucrose, 0.85 mg/L silver nitrate, 0.1 nM acetosyringone, and 3.0 g/L Gelrite, pH 5.8. Embryos are incubated axis down, in the dark for 3 days at 20° C., then incubated 4 days in the dark at 28° C., then transferred onto new media plates containing 4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.69 g/L L-proline, 30.0 g/L sucrose, 0.5 g/L MES buffer, 0.85 mg/L silver nitrate, 3.0 mg/L Bialaphos, 100 mg/L carbenicillin, and 6.0 g/L agar, pH 5.8. Embryos are subcultured every three weeks until transgenic events are identified. Somatic embryogenesis is induced by transferring a small amount of tissue onto regeneration medium (4.3 g/L MS salts (Gibco 11117), 5.0 ml/L MS Vitamins Stock Solution, 100 mg/L myo-inositol, 0.1 ?M ABA, 1 mg/L IAA, 0.5 mg/L zeatin, 60.0 g/L sucrose, 1.5 mg/L Bialaphos, 100 mg/L carbenicillin, 3.0 g/L Gelrite, pH 5.6) and incubation in the dark for two weeks at 28° C. All material with visible shoots and roots are transferred onto media containing 4.3 g/L MS salts (Gibco 11117), 5.0 ml/L MS Vitamins Stock Solution, 100 mg/L myo-inositol, 40.0 g/L sucrose, 1.5 g/L Gelrite, pH 5.6, and incubated under artificial light at 28° C. One week later, plantlets are moved into glass tubes containing the same medium and grown until they were sampled and/or transplanted into soil.

### Ribonucleoprotein Transformation

The components comprising the Type I cascade and associated guide polynucleotide(s) ribonucleoprotein (RNP) complex can be recombinantly expressed and purified. RNP complex assembly can be carried-out directly in the cell recombinantly expressing the components (Sinkunas, T. et al. (2013) EMBO J. 32:385-394). Following purification, the RNP complex(es) can be delivered by particle gun transformation as described in Svitashev, S. et al. (2016) Nat. Commun. 7:13274. Briefly, RNPs (and optionally DNA expression) are precipitated onto 0.6 mm (average diameter) gold particles (Bio-Rad, USA) using a water soluble cationic lipid TranslT-2020 (Minis, USA) as follows: 50 ml of gold particles (water suspension of 10 mg/ml) and 2 ml of TransIT-2020 water solution are added to the premixed RNPs (and optionally DNA expression vectors), mixed gently, and incubated on ice for 10 min. RNP/DNA-coated gold particles are then pelleted in a microfuge at 8,000 g for 30 s and supernatant is removed. The pellet is then resuspended in 50 ml of sterile water by brief sonication. Immediately after sonication, coated gold particles are loaded onto a microcarrier (10 ml each) and allowed to air dry. Immature maize embryos, 8-10 days after pollination, are then bombarded using a PDS-1000/He Gun (Bio-Rad, USA) with a rupture pressure of 425 pounds per inch square. Post-bombardment culture, selection, and plant regeneration are performed as previously described above.

### Variations in Delivery

Type I-E cascade, guide polynucleotide, and optionally associated nuclease can be delivered as DNA expression cassettes, RNA, messenger RNA (5′-capped and polyadenylated), or protein or combinations thereof. Cell lines or transformants can also be established stably expressing all but one or more of the components needed to form a functional guide polynucleotide/Cas complex so that upon delivery of the missing component(s) a functional guide polynucleotide/Cas complex can form.

### Sequence Verification of Genomic Polynucleotide Modification

Samples of a transformed plant are obtained and sequenced via any method known in the art, and compared to the genomic sequences of an isoline plant not transformed with the Type I-E cascade, guide polynucleotide, and associated nuclease. The presence of non-homologous end-joining (NHEJ) insertion and/or deletion (indel) mutations resulting from DNA repair can also be used as a signature to detect cleavage activity.

### Example 4: Demonstration of Type I-E Cascade Complex Formation in Maize Cells

In this example, methods for examining the functional formation of a Type I-E cascade and guide RNA complex in eukaryotic cells, particularly plant cells, are described.

In one method, the functional formation of a Type I cascade and guide RNA(s) were monitored by an immunofluorescent gene activation assay. This was accomplished by constructing a DNA expression cassette in such a way that in the absence of cascade, guide RNA complex formation no or very basal expression of a fluorescent protein would be detected while in the presence of cascade guide RNA complex formation a gene encoding a fluorescent protein would be activated resulting in detectable fluorescence. This DNA expression cassette comprised an upstream region where cascade and guide RNA complex(es) may bind (SEQ ID NO:104), a minimal 35S promoter from the cauliflower mosaic virus (CMV) (SEQ ID NO:105), a 5 prime untranslated region (UTR) from the tobacco mosaic virus (TMV) (SEQ ID NO:106), a region encoding the Ds-Red Express fluorescent protein (BD Bioscience) (SEQ ID NO:107) and a rice Ubiquitin terminator (SEQ ID NO:108). The key elements of this gene activation (GACT) DNA expression cassette are illustrated in FIG. 9 and sequence disclosed in SEQ ID NO:109.

Next, the concentration of GACT that provides minimal background fluorescence was established. This was accomplished by precipitating different concentrations (1 ng, 15 ng and 25 ng) of GACT onto gold particles (as described in Example 3), performing particle gun transformation (as described in Example 3) and examining the transformed immature maize embryos (IMEs) for Ds-Red expression using a fluorescent microscope (Nikon AZ100 (Nikon, Japan)) two days following transformation. Images were captured using a Nikon Digital Sight Ds-Fi1 camera (Nikon, Japan) and NIS-Elements BR software (version 4.00.07) (Nikon, Japan). The delivery of 1 ng and 15 ng of GACT by particle gun yielded low levels of background Ds-Red fluorescence so 15 ng was selected for use in future experiments.

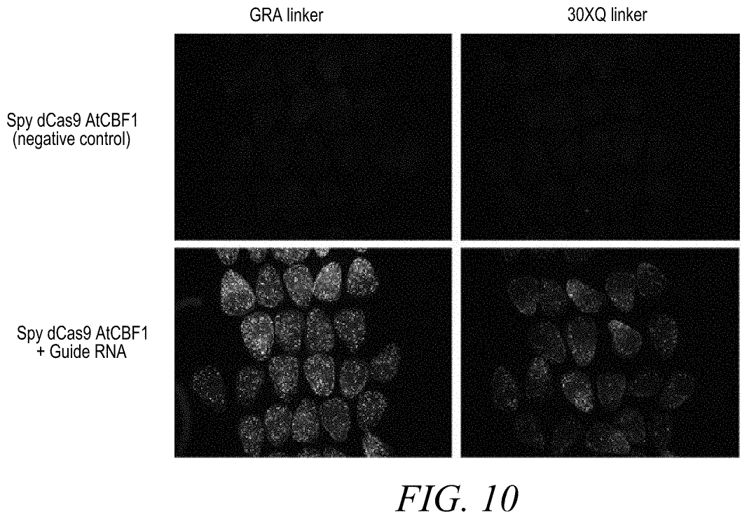

Next, the ability to induce gene activation was confirmed by utilizing a Streptococcus pyogenes (Spy) dCas9 linked AtCBF1 expression cassettes (see Example 2) and Spy Cas9 guide RNA transcriptional cassettes constructed as described in US patent application WO 2015026883 A1 except the variable targeting domains were designed to direct the Spy dCas9 linked AtCBF1 to bind to 4 target sites 5 prime of the 35S CMV promoter in the GACT expression cassette. Particle gun transformation experiments were performed as described in Example 3, except immature embryos were harvested 2-4 days following transformation to evaluate gene activation. Transformation experiments were assembled in triplicate and triplicate and a visual marker DNA expression cassette encoding a cyan fluorescent protein (CFP) was also co-delivered to aid in the selection of evenly transformed immature embryos. Negative controls consisted of experiments conducted in the absence of a guide RNA. As shown in FIG. 10, both Spy dCas9 linked AtCBF1 expression cassettes produced detectable levels of fluorescence, however, the cassette where the Spy dCas9 and AtCBF1 protein were linked by a sequence encoding the GRA linker produced the highest frequency of gene activation.

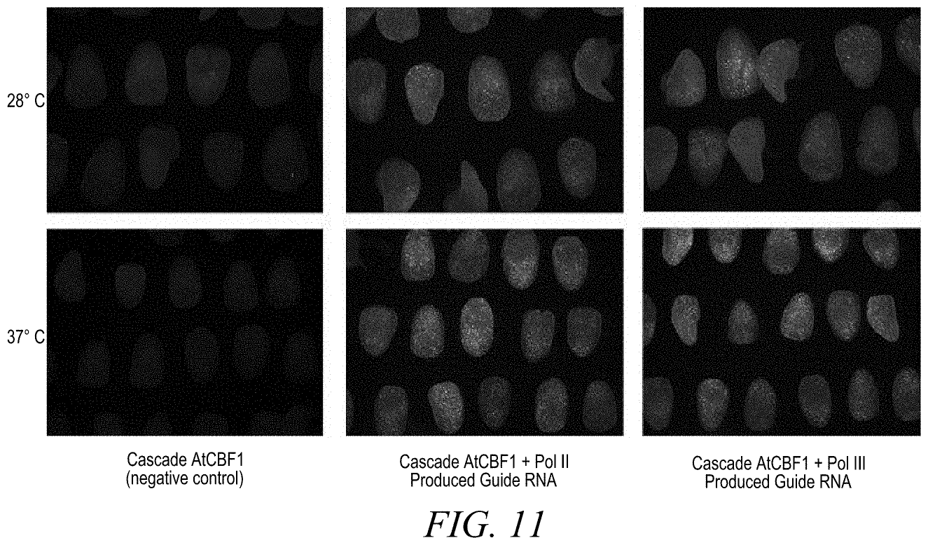

Next, the ability to form and then direct cascade guide RNA complexes to DNA target sites in maize was assessed. Initially, the combination (and concentration) of cascade and guide RNA expression constructs listed in Table 9 were tested for their ability to activate gene expression. Out of the 6 combinations tested, one (Treatment 4, Table 9) seemed to induce low levels of Ds-Red gene activation over the negative controls (transformations assembled without a guide RNA). Next the effect of both temperature and guide RNA transcriptional cassette promoter (either polymerase II or polymerase III) (see Example 2) were examined. For the temperature treatment, transformation experiments were setup in duplicate with one set of embryos placed at 28° C. (typical for maize particle gun transformation) and the other set was placed immediately at 37° C. until it was evaluated two days later for Ds-Red gene activation. Transformation experiments were performed with the combination of cascade expression cassettes that provided an initial indication of gene activation (Treatment 4, Table 9) plus one other treatment incorporating the guide RNA transcriptional cassette utilizing the polymerase III promoter (SEQ ID NO:77). Surprisingly, Ds-Red gene activation was clearly noticeable in the maize embryos that had been incubated at 37° C. compared to the 28° C. temperature treatment (FIG. 11). Additionally, the promoter driving the guide RNA transcriptional cassette (either polymerase II or polymerase III) yielded near equivalent levels of Ds-Red fluorescence (FIG. 11).

TABLE 9
Combination and concentration of Streptococcus thermophilus DGCC7710 Type I-E
DNA expression cassettes co-precipitated onto gold particles

| | | | | | | | | | | | | Polymerase | | |
|---|---|---|---|---|---|---|---|---|---|---|---|---|---|---|
| | | | | | | | | | | | | II Guide RNA | GACT | |
| | Cascade Expression Cassette (SEQ ID NOs) | | | | | | | | | | | (SEQ ID | (SEQ ID | Visual |
| Treatment | 44 | 46 | 47 | 48 | 49 | 69 | 70 | 71 | 72 | 73 | 74 | NO: 80) | NO: 109) | Marker |
| | DNA Concentration (ng) Co-Precipitated onto Gold Particles | | | | | | | | | | | | | |
| 1 | — | 15 | 15 | 40 | 15 | 15 | — | — | — | — | — | 30 | 15 | 20 |
| 2 | — | 15 | 15 | 40 | 15 | — | 15 | — | — | — | — | 30 | 15 | 20 |
| 3 | — | 15 | 15 | 40 | 15 | — | — | 15 | — | — | — | 30 | 15 | 20 |
| 4 | — | 15 | 15 | 40 | 15 | — | — | — | 15 | — | — | 30 | 15 | 20 |
| 5 | 15 | — | 15 | 40 | 15 | — | — | — | — | 15 | — | 30 | 15 | 20 |
| 6 | 15 | — | 15 | 40 | 15 | — | — | — | — | — | 15 | 30 | 15 | 20 |

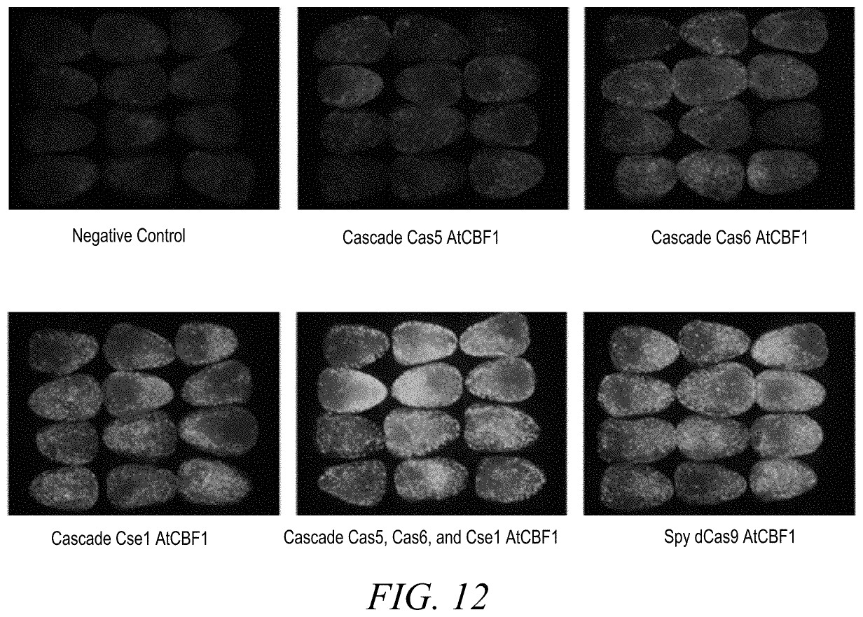

To further optimize the cascade complex for gene activation, the sequence encoding the AtCBF1 transcriptional activator motif (SEQ ID NO:50) was maize codon optimized and linked to the carboxyl termini (C-termini) of the cas6 and cse1 genes (SEQ ID NOs: 20 and 22, respectively) with a sequence encoding a SV40 NLS (SEQ ID NO:18) and ‘GRA’ linker being incorporated between the cas6 and cse1 genes and the AtCBF1 coding region. The resulting genes (SEQ ID NOs: 110 and 111) were then placed into a expression cassette resulting in SEQ ID NOs: 112 and 113 and tested for their gene activation potential as described above with the following exceptions. First, a new gene activation construct (GACT2) was built that contained just a single Spy dCas9 and cascade binding site (SEQ ID NO:114). Second, U6 polymerase III expression constructs encoding a Spy dCas9 and cascade guide RNA (SEQ ID NOs: 115 and 116, respectively) capable of targeting this new site were generated. A treatment assembled in the absence of a guide RNA served as a negative control while the Spy dCas9 linked (GRA) AtCBF1 (SEQ ID NO:76) and associated guide RNA (SEQ ID NO:115) expression constructs were used as a positive control. To enable a comparison with the Sth7710 cascade gene activation results obtained previously, Treatment 4 (Table 9) was also included. To test for an additive effect, cas5, cash, and cse1 linked AtCBF1 genes were also combined in a single treatment. DNA concentration of each cascade expression construct co-precipitated onto gold particles was as described in Table 10 and biolistic transformation was performed as described above. As shown in FIG. 12, Ds-Red gene activation was enhanced when AtCBF1 was linked to the C-termini of Cas6 and Cse1 relative to Cas5 (Treatment 4 (Table 10A)). Moreover, when combined, AtCBF1 linked Cas5, Cas6, and Cse1 Ds-Red gene activation visually surpassed that generated by Spy dCas9 linked (GRA) AtCBF1. Next, the amount of Ds-Red protein expressed was quantified using an Octet RED96 System (ForteBio, USA) as follows. Total protein was extracted from immature maize embryos (IMEs) two days after transformation and normalized to 1300 μg/mL. Then, a standard curve was generated by examining the binding rate (BR) of purified Ds-Red protein (Clontech) at known concentrations (500, 100, 25, 6.25 ng/mL) to a Ds-Red specific biosensor (Anti-Human IgG Fc coated with a DsRed antibody (Clontech)). BRs of the standards were then utilized to generate a standard curve and calculate the concentration of Ds-Red in the transformed IMEs. Each sample was measured in triplicate. As shown in Table 11, quantification of Ds-Red protein present in the transformed tissue further corroborated the fluorescence imaging.

Taken together, data indicate that Type I-E cascade and guide RNA may be engineered to form a functional complex in eukaryotic cells, particularly plant cells, and be used for plant gene transcriptional activation. Additionally, our data also indicates that the multi-subunit nature of the cascade complex provides the basis for a robust RNA guided platform for genome modification where multiple tools (e.g. polynucleotides, domains, motifs, and/or fusions resulting in transcriptional activation, transcriptional repression, cleavage, nicking, DNA methylation, chromatin modification, deamination, and others) may be affixed.

TABLE 10
Combination and concentration of Streptococcus thermophilus DGCC7710 Type I-E
DNA expression cassettes co-precipitated onto gold particles for optimization experiments

| | | | | | | | | | Polymerase | | |
|---|---|---|---|---|---|---|---|---|---|---|---|
| | | | | | | | | | II Guide RNA | GACT | |
| | Cascade Expression Cassette (SEQ ID NOs) | | | | | | | | (SEQ ID | (SEQ ID | Visual |
| Treatment | 44 | 46 | 47 | 48 | 49 | 72 | 112 | 113 | NO: 116) | NO: 114) | Marker |
| | DNA Concentration (ng) Co-Precipitated onto Gold Particles | | | | | | | | | | |
| 1 | — | 20 | 20 | 45 | 20 | 20 | — | — | 30 | 15 | 20 |
| 2 | 20 | 20 | — | 45 | 20 | — | 20 | — | 30 | 15 | 20 |
| 3 | 20 | — | 20 | 45 | 20 | — | — | 20 | 30 | 15 | 20 |
| 4 | — | — | — | 45 | 20 | 20 | 20 | 20 | 30 | 15 | 20 |

TABLE 11
Quantification of the amount of Ds-Red expressed in
S. thermophilus DGCC7710 Type I-E cascade and
S. pyogenes dCas9 gene activation experiments

| | Average | |
|---|---|---|
| | Ds-Red | |
| | Concentration | Standard Error |
| Treatment | (ppm) | Measurement |
| Cascade No Guide RNA | 5.2 | 1.4 |
| Cascade Cas5 AtCBF1 | 15.4 | 2.6 |
| Cascade Cas6 AtCBF1 | 21.2 | 3.2 |
| Cascade Cse1 AtCBF1 | 34.2 | 4.9 |
| Cascade Cas5, Cas6, Cse1 AtCBF1 | 102.2 | 11.7 |
| Spy dCas9 No Guide RNA | 10.0 | 1.1 |
| Spy dCas9 AtCBF1 | 54.7 | 5.3 |

### Example 5: Type I-E Cascade-Mediated Transcriptional Manipulation of a Eukaryotic Gene

In this example, the transcriptional activation of a eukaryotic, in particular plant, chromosomal gene with an engineered Type I-E cascade guide polynucleotide complex is described.

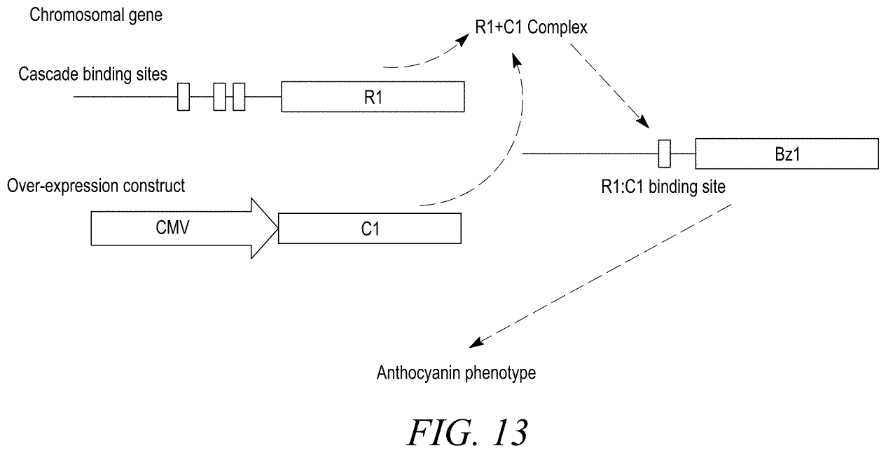

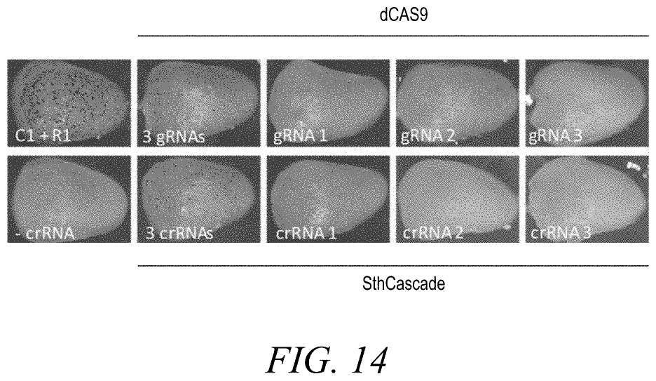

Since anthocyanin pigmentation may be used as a quantitative visual marker (Ludwig, S. R. et al. (1990) Science. 247: 449-450) for maize transformation, it was selected as a target for Sth7710 cascade endogenous gene activation. In the aleurone cell layer, maize anthocyanin pigmentation is activated through the coordinated action of two transcription factors R1 and C1 (Chandler, V. L. et al. (1989) Plant Cell. 1: 1175-1183, Grotewold, E. et al. (2000) PNAS. 97: 13579-13584 and Sharma, M. et al. (2011) Genetics. 188:69-79). Therefore, to induce anthocyanin production using Sth7710 cascade, the endogenous R1 gene was targeted for transcriptional up-regulation by cascade while over-expressing the C1 gene (FIG. 13) (alternate strategies include over-expression of the R1 gene combined with C1 gene activation, transcriptional activation of both the R1 and C1, and direct activation of Bzl). R1 gene activation was accomplished by first selecting 3 protospacer targets (adjacent to an appropriate PAM) in the promoter region of the R1 gene (see Table 12) (SEQ ID NOs: 117-119) and constructing U6 polymerase III expression constructs encoding Sth7710 cascade guide RNAs capable of targeting R1 (SEQ ID NOs: 123-125). Next, a C1 over-expression cassette was generated by operably linking the Cl coding DNA sequence (CDS) (SEQ ID NO:126) with a Cauliflower Mosaic Virus 35S (CAMV35S) enhancer (SEQ ID NO:127), Cauliflower Mosaic Virus (CMV) promoter (SEQ ID NO:128), TMV 5 prime UTR (SEQ ID NO:106), alcohol dehydrogenase 1 (ADH1) intron 1 (SEQ ID NO:129), and a terminator resulting in a chimeric Cl gene over-expression cassette (SEQ ID NO:130). For use as a positive control, a DNA expression cassette capable of over-expressing the R1 CDS was also generated (SEQ ID NO:132). It comprised a ubiquitin (UBI) promoter (SEQ ID NO:36), UBI 5′ UTR (SEQ ID NO:37), UBI intron 1 (SEQ ID NO:38), R1 CDS (SEQ ID NO:131), and a terminator. Next, biolistic transformation was carried-out as described in Example 3 using the expression constructs and associated concentrations (for loading onto gold particles) listed in Table 13. Paired with the C1 over-expression construct, each of the 3 guide RNAs for Sth7710 cascade targeting R1 were evaluated individually and in combination for their ability to upregulate anthocyanin production. To compare gene activation with Spy dCas9, three Spy dCas9 protospacer targets were selected in the promoter of the R1 gene (see Table 12) (SEQ ID NOs: 120-122) and U6 polymerase III transcriptional cassettes were generated (SEQ ID NOs: 133-135). To promote robust U6 polymerase III transcription, a G nucleotide (nt) was included at the beginning of the guide RNA if the Spy dCas9 protospacer target did not naturally start with one. Delivery of both R1 and C1 over-expression cassettes served as a positive control while experiments omitting the guide RNA expression construct provided a negative control. As shown in FIG. 14, an anthocyanin phenotype was observed for both Sth7710 cascade and Spy dCas9. Experiments utilizing all 3 guide RNAs, produced the highest levels of anthocyanin induction with cascade and Spy dCas9 yielding near equivalent signal (FIG. 14). By quantifying maize cells with an anthocyanin cellular phenotype (across 3 biological replicates) (Table 14), we further substantiate the finding that a Type I cascade complex may be used as a tool for manipulating gene expression in Zea mays.

TABLE 12
Maize chromosomal DNA targets selected in the
promoter region of the R1 gene

| | Location | | | SEQ | |
|---|---|---|---|---|---|
| | (B73 | PAM | | ID | PAM |
| Name | RefGen_v4) | (5′) | Protospacer Target | NO. | (3′) |
| A2+ | Chr 10: | AA | GAGGGTCTACTTCCATCA | 117 | — |
| | 139780885- | | CCGTCTTGCTCGGTC | | |
| | 139780919 | | | | |
| A4+ | Chr 10: | AA | CAGCAGTAGTGTTACAGA | 118 | — |
| | 139780833- | | AGCTAAACTCAACCA | | |
| | 139780867 | | | | |
| A1− | Chr 10: | AA | TTTATGGACAGAGCTCCA | 119 | — |
| | 139780942- | | AGTGACCGAGCAAGA | | |
| | 139780908 | | | | |
| A3+ | Chr 10: | — | GAGCTCCACCAAAGACAA | 120 | AGG |
| | 139780868- | | AG | | |
| | 139780890 | | | | |
| A7+ | Chr 10: | — | CAGTGCTATAATATGAGT | 121 | TGG |
| | 139780671- | | GG | | |
| | 139780693 | | | | |
| A2− | Chr 10: | — | CTCCAAGTGACCGAGCAA | 122 | CGG |
| | 139780927- | | GA | | |
| | 139780905 | | | | |

TABLE 13
Combination and concentration of Streptococcus thermophilus DGCC7710 Type I-E
DNA expression cassettes co-precipitated onto gold particles for endogenous gene activation
experiments

| | | | | | | | | | Polymerase III Guide | | | C1 Over- |
|---|---|---|---|---|---|---|---|---|---|---|---|---|
| | | | | | | | | | RNA Cassette (SEQ ID | | | Expression |
| | Cascade Expression Cassette (SEQ ID NOs) | | | | | | | | NOs) | | | Cassette (SEQ |
| Treatment | 44 | 46 | 47 | 48 | 49 | 72 | 112 | 113 | 123 | 124 | 125 | ID NO: 130) |
| | DNA Concentration (ng) Co-Precipitated onto Gold Particles | | | | | | | | | | | |
| 1 | — | — | — | 45 | 20 | 20 | 20 | 20 | — | — | — | 40 |
| 2 | — | — | — | 45 | 20 | 20 | 20 | 20 | 20 | — | — | 40 |
| 3 | — | — | — | 45 | 20 | 20 | 20 | 20 | — | 20 | — | 40 |
| 4 | — | — | — | 45 | 20 | 20 | 20 | 20 | — | — | 20 | 40 |
| 5 | — | — | — | 45 | 20 | 20 | 20 | 20 | 6.67 | 6.67 | 6.67 | 40 |

TABLE 14
Quantification of the number of anthocyanin “red”
cells in S. thermophilus DGCC7710 Type I-E cascade and
S. pyogenes dCas9 gene activation experiments

| | Average | |
|---|---|---|
| | Anthocyanin | Standard |
| | Positive | Error |
| Treatment | Cells | Measurement |
| Spy dCas9 AtCBF1 | 0 | 0 |
| No Guide RNA | | |
| Spy dCas9 AtCBF1 | 124.7 | 39.3 |
| Plus Guide RNAs Targeting R1 | | |
| Cascade Cas5, Cas6, | 0 | 0 |
| Cse1 AtCBF1 No Guide RNA | | |
| Cascade Cas5, Cas6, Cse1 | 152.7 | 18.9 |
| AtCBF1 Plus Guide RNAs Targeting R1 | | |

### Example 6: Type I-E Cascade-Mediated Cleavage of Eukaryotic Cellular DNA

In this example, the cleavage of DNA in eukaryotic, in particular plant, cells with an engineered Type I-E cascade guide polynucleotide complex is described.

Cascade by itself does not cleave DNA but only recognizes and binds its DNA target. To enable DNA cleavage, it can be combined with the exonuclease naturally associated with the CRISPR-Cas system, Cas3, or other nuclease(s) or nuclease domains (for example but not limited to a nuclease domain from a Type II restriction endonuclease (i.e., FokI (Kim, Y. et al. (1996) Proc. Natl. Acad. Sci. USA. 93:1156-1160)) or a nuclease domain from a homing endonuclease (i.e., I-Teel (Kleinstiver, B. et al. (2012) Proc. Natl. Acad. Sci. USA. 109:8061-8066)). Nucleases or nuclease domains may be recruited, associated with, linked to, fused to, assembled with or expressed as part of the cascade complex. Optionally, if a nickase (for example but not limited to an engineered Cas3 nickase (see Example 2)) or nuclease that requires dimerization (for example but not limited to a Type II restriction endonuclease (i.e. FokI (Kim, Y. et al. (1996) Proc. Natl. Acad. Sci. USA. 93:1156-1160) is utilized, two or more DNA target sites in close proximity and on opposite DNA strands or combinations thereof can be targeted to generate DNA double-strand break(s) (DSB(s)).

Once cleavage enabled, the cascade complex can be directed to DNA target sites by a guide RNA and monitored for the presence of insertion and deletion (indel) mutations indicative of target site cleavage and cellular non-homologous end-joining (NHEJ) DNA repair. This is carried-out by targeted deep sequencing as described in Karvelis, T. et al. (2015) Genome Biology. 16:253 (Methods Section: In planta mutation detection). Briefly, after 2 days, the 20-30 most evenly transformed immature maize embryos (IMEs) are harvested based on their fluorescence. Total genomic DNA is extracted and the region surrounding the intended target site is PCR amplified with Phusion® HighFidelity PCR Master Mix (New England Biolabs, M0531L) adding on the sequences necessary for amplicon-specific barcodes and Illumina sequencing using “tailed” primers through two rounds of PCR and deep sequenced. The resulting reads are examined for the presence of mutations at the expected site of cleavage by comparison to control experiments where the small RNA transcriptional cassette is omitted from the transformation.

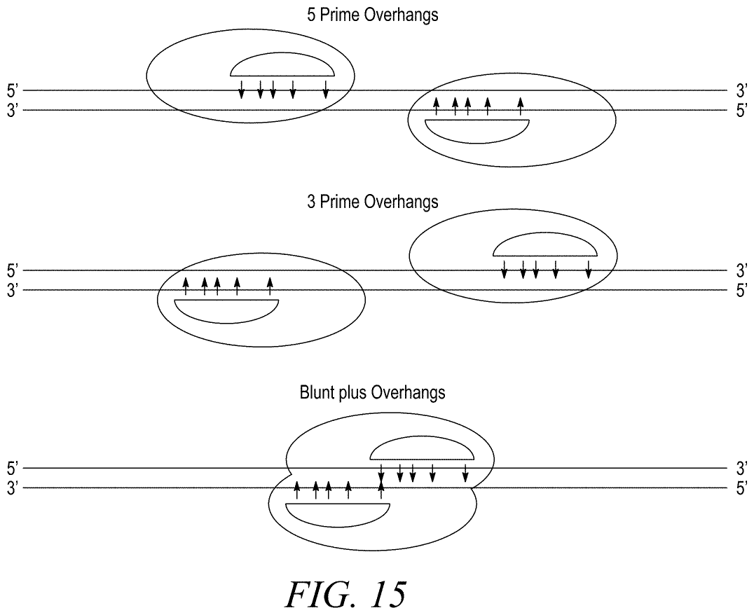

As illustrated in FIG. 15, experiments were performed using cooperative nicking with an engineered Cas3 nickase (Example 2) and two cascade targets in close proximity (on opposite DNA strands). As shown in FIG. 16, this resulted in targeted chromosomal mutagenesis with the predominant repair of the DSB being large deletions (>10 bp) of variable length that typically originated within one of the cascade target sequences although small deletion (1 or 2 bp) mutations were also detected. In total, 4 pairs of cascade target sites (A2+/A1−, A2+/A3−, A4+/A1−, and A4+/A3−) (Table 15), varying both in distance from each other and target site orientation, were selected for testing with the Cas3 nickase. As shown in Table 15, all produced chromosomal mutations unique to the Cas3 nickase treatment, illustrating the target design flexibility of the cooperative cascade-Cas3 nickase approach described herein. Additionally, the mechanism of generating a DSB provided by an engineered Cas3 nickase is distinct from other genome editing tools: it results in more than one nicking event in a strand-specific manner (FIG. 15). This feature is novel and can be leveraged to enhance DNA repair outcomes. For example, the creation of multiple nicks on one strand would likely be acted on by repair processes to generate a single strand gap or gaps between adjacent nicks. Multiple nicks on both strands of a segment of DNA could then lead to a novel intermediate in which opposing gaps would be the equivalent of a double-strand break with 3′ or 5′ overhangs depending on orientation of the cascade targets. Both sets of overhangs would not be optimal substrates for NHEJ repair and 3′ overhangs in particular could be primarily acted upon by homologous recombination which normally uses 5′to 3′ exonucleases to initiate single strand formation. U6 polymerase III crRNA transcriptional constructs were assembled as described in Example 2. Transformation experiments were assembled using particle gun transformation as described in Example 3 except immature embryos were harvested 2-4 days following transformation. DNA expression constructs co-delivered onto gold particles was as listed in Table 16. Experiments assembled in the absence of a guide RNA served as a negative control. Mutations were detected by Illumina deep sequencing as described above.

TABLE 15
Pairs of Streptococcus thermophilus DGCC7710 cascade chromosomal
protospacer targets selected in the promoter region of the R1 gene
and associated mutation frequencies when combined with a Cas3 nickase

| | | Protospacer | | | |
|---|---|---|---|---|---|
| | Location of Protospacer and | Target SEQ ID | Unique | | |
| | PAM, Respectively (B73 | NOs, | Mutant | Total | % Mutant |
| Pair | RefGen_v4) | Respectively | Reads | Reads | Reads |
| A2+/A1− | Chr 10: 139780885- | 117/119 | 44 | 203,351 | 0.02% |
| | 139780919/ | | | | |
| | Chr 10: 139780942- | | | | |
| | 139780908 | | | | |
| A2+/A3− | Chr 10: 139780885- | 117/163 | 1,016 | 1,598,106 | 0.06% |
| | 139780919/ | | | | |
| | Chr 10: 139780847- | | | | |
| | 139780813 | | | | |
| A4+/A1− | Chr 10: 139780833- | 118/119 | 388 | 661,310 | 0.06% |
| | 139780867/ | | | | |
| | Chr 10: 139780942- | | | | |
| | 139780908 | | | | |
| A4+/A3− | Chr 10: 139780833- | 118/163 | 186 | 663,242 | 0.03% |
| | 139780867/ | | | | |
| | Chr 10: 139780847- | | | | |
| | 139780813 | | | | |

TABLE 16
Combination and concentration of Streptococcus thermophilus DGCC7710 Type I-E
DNA expression cassettes co-precipitated onto gold particles for experiments targeting
chromosomal sites with Cas3 nickase

| | | | | | | Polymerase III Guide | | | | Cas3 Nickase | |
|---|---|---|---|---|---|---|---|---|---|---|---|
| | Cascade Expression Cassette | | | | | RNA Cassette (SEQ ID | | | | Expression | |
| | (SEQ ID NOs) | | | | | NOs) | | | | Cassette (SEQ | Visual |
| Treatment | 44 | 46 | 47 | 48 | 49 | 123 | 124 | 125 | 164 | ID NO: 101) | Marker |
| | DNA Concentration (ng) Co-Precipitated onto Gold Particles | | | | | | | | | | |
| Ctrl | 20 | 20 | 20 | 45 | 20 | — | — | — | — | 50 | 20 |
| A2+/A1− | 20 | 20 | 20 | 45 | 20 | 30 | — | 30 | — | 50 | 20 |
| A2+/A3− | 20 | 20 | 20 | 45 | 20 | 30 | — | — | 30 | 50 | 20 |
| A4+/A1− | 20 | 20 | 20 | 45 | 20 | — | 30 | 30 | — | 50 | 20 |
| A4+/A3− | 20 | 20 | 20 | 45 | 20 | — | 30 | — | 30 | 50 | 20 |

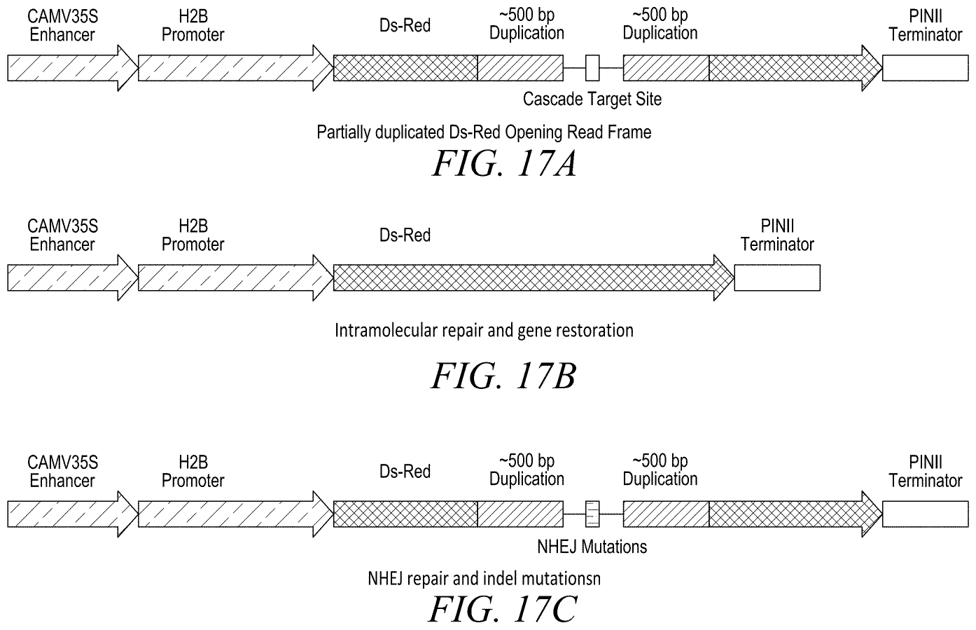

DNA cleavage can also be monitored using an immunofluorescent assay. For this, the gene encoding the Ds-Red Express fluorescent protein (SEQ ID NO:107) may be partially duplicated and a cascade target sequence can be incorporated between the duplicated fragments (FIG. 17A). To boost expression, the 5 prime end of the partially duplicated DsRed gene is operably linked to the cauliflower mosaic virus 35S (CAMV35S) enhancer (SEQ ID NO:127) and Maize Histone 2B (H2B) promoter (SEQ ID NO:192) and the 3 prime end fused with the Potato Proteinase Inhibitor II (PINII) terminator (SEQ ID NO:193) resulting in a Ds-target site-sRed reporter construct illustrated in FIG. 17A. With this configuration, cleavage and cellular repair of the target sequence will result in two outcomes. The first outcome is intramolecular DNA repair between the duplicated regions of the Ds-Red gene. This restores the gene resulting in Ds-Red expression and immunofluorescence (FIG. 17B). The second outcome produces indel mutations indicative of DNA target cleavage and repair by the non-homologous end-joining (NHEJ) pathway that can be detected by targeted deep sequencing as described in Karvelis, T. et al. (2015) Genome Biology. 16:253 (Methods Section: in planta mutation detection) (FIG. 17C). When delivered into eukaryotic cells, either DNA repair outcome may be recovered permitting an immunofluorescence visual screen followed by a confirmatory sequencing step to detect DNA target cleavage activity.

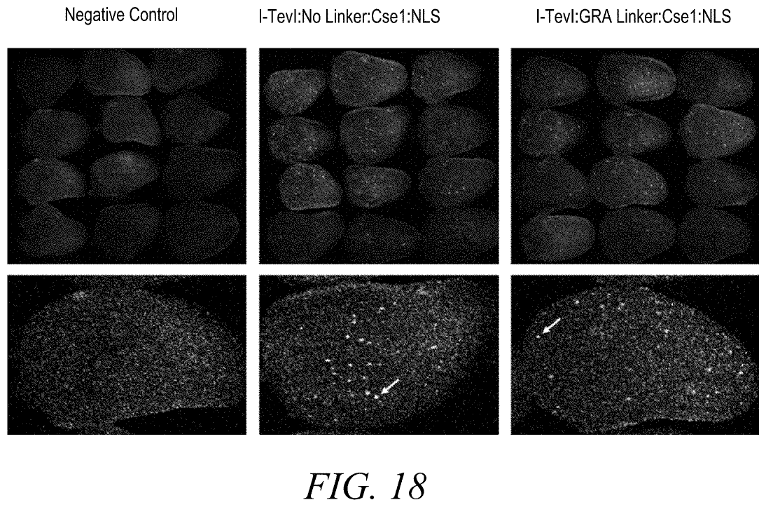

As illustrated in FIG. 18, expression cassettes encoding I-Teel fused to the 5 prime end of the cse1 gene (SEQ ID NOS: 187 and 188) produced Ds-Red immunofluorescent signal, indicative of target cleavage, intramolecular repair, and functional restoration of the Ds-Red gene. Examination of the cascade target sites by deep sequencing yielded small insertion or deletion mutations originating within the cleavage sites of the I-Teel cleavage motif, CNNNG (where N represents any base pair A, T, G, or C), indicative of NHEJ DNA repair further collaborating the fluorescent imaging data (FIGS. 19 and 20). Mutations demonstrative of DNA target cleavage and NHEJ repair were also recovered for DNA expression constructs (SEQ ID NOs: 185 and 186) where the I-TevI cleavage domain was appended to the 5 prime end of the cash gene (FIGS. 21 and 22). U6 polymerase III Sth7710 crRNA transcriptional constructs targeting the Ds-target site-sRed reporter construct were assembled as described in Example 2. Transformation experiments were assembled using particle gun transformation as described in Example 3 expect immature embryos were harvested 2-4 days following transformation. Images were captured using a Nikon Digital Sight Ds-Fi1 camera (Nikon, Japan) and NIS-Elements BR software (version 4.00.07) (Nikon, Japan). Experiments assembled with a Ds-Red reporter that did not contain a cascade target site (Ds:no target site:sRed) served as a negative control. Mutations were detected by Illumina deep sequencing as described above.

Taken together, data indicate that the Type I-E cascade may be engineered to cleave cellular DNA in eukaryotic cells, particularly plant cells. This feature permits natural cellular DNA repair processes to be harnessed enabling the modification of an organism's genetic code. This includes but is not limited to the introduction of NHEJ mutations, excision of chromosomal DNA fragments comprised of either transgenic or endogenous DNA, editing of the codon composition of native or transgenic genes by homologous recombination repair with a donor DNA repair template(s) (endogenous or exogenously supplied), and the site specific insertion of transgenic or endogenous DNA sequences by homologous recombination repair with a donor DNA repair template (endogenous or exogenously supplied). Thus, enabling Type I-E cascade genome editing.

Some aspects are listed as follows:

Aspect 1: A method for forming a CRISPR cascade and guide polynucleotide complex in a eukaryotic cell, comprising providing to said eukaryotic cell: (a) a Cse2 polypeptide, and (b) a guide polynucleotide comprising a variable targeting domain that is substantially complementary to a polynucleotide target sequence in the genome of the eukaryotic cell; and incubating said eukaryotic cell; wherein said Cse1 polypeptide functionally associates with at least one other protein to form a cascade; and wherein the guide polynucleotide and cascade form a complex capable of recognizing and binding to the polynucleotide target sequence in the genome of the eukaryotic cell.

Aspect 2: A method for forming a CRISPR cascade and guide polynucleotide complex in a eukaryotic cell, comprising providing to said eukaryotic cell: (a) a cse1 polynucleotide, and (b) a guide polynucleotide comprising a variable targeting domain that is substantially complementary to a polynucleotide target sequence in the genome of said eukaryotic cell; and incubating said cell; wherein said cse1 polynucleotide is operably linked to a noncoding regulatory element rendering it capable of expressing a Cse1 polypeptide, and wherein said Cse1 polypeptide functionally associates with at least one other protein to form a cascade; and wherein said cascade and guide polynucleotide form a complex capable of recognizing and binding to a polynucleotide target sequence in the genome of said eukaryotic cell.

Aspect 3: The method of Aspect 1 or 2, wherein said cascade further comprises a protein selected from the group consisting of: Cse2, Cas7, Cas5, and Cas6.

Aspect 4: The method of Aspect 1 or 2, wherein said cascade further comprises at least two proteins selected from the group consisting of: Cse2, Cas7, Cas5, and Cas6.

Aspect 5: The method of Aspect 1 or 2, wherein said cascade further comprises at least three proteins selected from the group consisting of: Cse2, Cas7, Cas5, and Cas6.

Aspect 6: The method of Aspect 1 or 2, wherein said cascade further comprises Cse2, Cas7, Cas5, and Cas6.

Aspect 7: The method of any of Aspects 2-6, wherein Cas5 is linked to a heterologous polypeptide, wherein said heterologous polypeptide is capable of a function selected from the group consisting of: transcription repression, transcription modulation, transcription activation, polynucleotide methylation, polynucleotide demethylation, polynucleotide cleavage, polynucleotide nicking, deamination, and a combination thereof.

Aspect 8: The method of Aspect 7, wherein the heterologous polynucleotide is linked to the C-terminal end of Cas5.

Aspect 9: The method of Aspect 7, wherein the Cas5 and the heterologous polynucleotide are linked by 10 amino acids.

Aspect 10: The method of Aspect 2, wherein a polynucleotide selected from the group consisting of: cse2, cas7, cas5, and cas6 is further introduced into said eukaryotic cell.

Aspect 11: The method of Aspect 2, wherein at least two polynucleotides selected from the group consisting of: cse2, cas7, cas5, and cas6 are further introduced into said eukaryotic cell.

Aspect 12: The method of Aspect 2, wherein at least three polynucleotides selected from the group consisting of: cse2, cas7, cas5, and cas6 are further introduced into said eukaryotic cell.

Aspect 13: The method of Aspect 2, wherein cse2, cas7, cas5, and cas6 are further introduced into said eukaryotic cell.

Aspect 14: The method of any of Aspects 2, 10, 11, 12, or 13, wherein at least one of the components is introduced into said eukaryotic cell via a recombinant DNA construct.

Aspect 15: The method of any of Aspects 2, 10, 11, 12, or 13, wherein at least one component has been optimized for expression in said eukaryotic cell.

Aspect 16: The method of Aspect 14, wherein the introduction into said eukaryotic cell is achieved via a method selected from the group consisting of: Agrobacterium-mediated transformation, particle bombardment, and floral dip.

Aspect 17: Any of the methods of Aspects 1-16, further comprising incubating said eukaryotic cell at a temperature of greater than 28 degrees Celsius.

Aspect 18: Any of the methods of Aspects 1-16, wherein the eukaryotic cell further comprises a guide polynucleotide comprising a sequence substantially complementary to a sequence within the genome of said eukaryotic cell.

Aspect 19: Any of the methods of Aspects 1-16, further comprising providing to the eukaryotic cell a guide polynucleotide comprising a sequence substantially complementary to a sequence within the genome of said eukaryotic cell.

Aspect 20: Any of the methods of Aspects 1-16, wherein said eukaryotic cell comprises an endonuclease.

Aspect 21: The method of Aspect 20, wherein said endonuclease is Cas3.

Aspect 22: Any of the methods of Aspects 1-16, further comprising providing to the eukaryotic cell an endonuclease.

Aspect 23: The method of Aspect 22, wherein said endonuclease is Cas3.

Aspect 24: The method of any of Aspects 20-23, further comprising nicking or cleaving at least one strand of said polynucleotide target sequence.

Aspect 25: Any of the methods of Aspects 1-24, wherein said eukaryotic cell is a plant cell.

Aspect 26: The method of Aspect 25, wherein said plant cell is a monocot plant cell.

Aspect 27: The method of Aspect 26, wherein said monocot plant cell is obtained or derived from a plant selected from the group consisting of: corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

Aspect 28: The method of Aspect 25, wherein said plant cell is a dicot plant cell.

Aspect 29: The method of Aspect 28, wherein said dicot plant cell is obtained or derived from a plant selected from the group consisting of: soybean (Glycine max), Brassica species (for example but not limited to: oilseed rape or Canola) (Brassica napus, B. campestris, Brassica rapa, Brassica juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum).

Aspect 30: A eukaryotic cell produced by any of the methods of Aspects 1-29.

Aspect 31: An organism or tissue derived from the eukaryotic cell of Aspect 30.

Aspect 32: A progeny of the organism of Aspect 31.

Aspect 33: A plant derived from the eukaryotic cell of Aspect 30.

Aspect 34: A progeny of the plant of Aspect 33.

Aspect 35: Any of the methods of Aspects 1-34, wherein said CRISPR cascade is a Type I-E cascade.

Aspect 36: Any of the methods of Aspects 1-34, wherein said Cse1 polypeptide or cse1 polynucleotide was derived from a host organism comprising a Type I-E CRISPR system.

Aspect 37: A method for modifying a polynucleotide target sequence in the genome of a eukaryotic cell, comprising providing to said eukaryotic cell: (a) a Cse1 polypeptide, and (b) a guide polynucleotide comprising a variable targeting domain that is substantially complementary to said polynucleotide target sequence in the genome of said eukaryotic cell; and incubating said cell; wherein said Cse1 polypeptide functionally associates with at least one other protein to form a cascade; wherein the guide polynucleotide and cascade form a complex capable of recognizing and binding to the polynucleotide target sequence in the genome of the eukaryotic cell; and wherein the polynucleotide target sequence is modified by at least one nucleotide addition, substitution, or deletion.

Aspect 38: A method for modifying a polynucleotide target sequence in the genome of a eukaryotic cell, comprising providing to said eukaryotic cell: (a) a cse1 polynucleotide, and (b) a guide polynucleotide comprising a variable targeting domain that is substantially complementary to said polynucleotide target sequence in the genome of said eukaryotic cell; and incubating said cell; wherein said cse1 polynucleotide is operably linked to a noncoding regulatory element rendering it capable of expressing a Cse1 polypeptide, and wherein said Cse1 polypeptide functionally associates with at least one other protein to form a cascade; wherein said cascade and guide polynucleotide form a complex capable of recognizing and binding to a polynucleotide target sequence in the genome of said eukaryotic cell; and wherein the polynucleotide target sequence is modified by at least one nucleotide addition, substitution, or deletion.

Aspect 39: The method of Aspect 35 or 38, wherein said cascade further comprises a protein selected from the group consisting of: Cse2, Cas7, Cas5, and Cas6.

Aspect 40: The method of Aspect 35 or 38, wherein said cascade further comprises at least two proteins selected from the group consisting of: Cse2, Cas7, Cas5, and Cas6.

Aspect 41: The method of Aspect 35 or 38, wherein said cascade further comprises at least three proteins selected from the group consisting of: Cse2, Cas7, Cas5, and Cas6.

Aspect 42: The method of Aspect 35 or 38, wherein said cascade further comprises Cse2, Cas7, Cas5, and Cas6.

Aspect 43: The method of any of Aspects 39-42, wherein Cas5 is linked to a heterologous polypeptide, wherein said heterologous polypeptide is capable of a function selected from the group consisting of: transcription repression, transcription modulation, transcription activation, polynucleotide methylation, polynucleotide demethylation, polynucleotide cleavage, polynucleotide nicking, deamination, and a combination thereof.

Aspect 44: The method of Aspect 43, wherein the heterologous polynucleotide is linked to the C-terminal end of Cas5.

Aspect 45: The method of Aspect 43, wherein the Cas5 and the heterologous polynucleotide are linked by 10 amino acids.

Aspect 46: The method of Aspect 38, wherein a polynucleotide selected from the group consisting of: cse2, cas7, cas5, and cas6 is further introduced into said eukaryotic cell.

Aspect 47: The method of Aspect 38, wherein at least two polynucleotides selected from the group consisting of: cse2, cas7, cas5, and cas6 are further introduced into said eukaryotic cell.

Aspect 48: The method of Aspect 38, wherein at least three polynucleotides selected from the group consisting of: cse2, cas7, cas5, and cas6 are further introduced into said eukaryotic cell.

Aspect 49: The method of Aspect 38, wherein cse2, cas7, cas5, and cas6 are further introduced into said eukaryotic cell.

Aspect 50: The method of any of Aspects 38, 46, 47, 48, or 49, wherein at least one of the components is introduced into said eukaryotic cell via a recombinant DNA construct.

Aspect 51: The method of any of Aspects 38, 46, 47, 48, or 49, wherein at least one of the components has been optimized for expression in said eukaryotic cell.

Aspect 52: The method of Aspect 50, wherein the introduction into said eukaryotic cell is achieved via a method selected from the group consisting of: Agrobacterium-mediated transformation, particle bombardment, and floral dip.

Aspect 53: Any of the methods of Aspects 35-52, further comprising incubating said eukaryotic cell at a temperature of greater than 28 degrees Celsius.

Aspect 54: Any of the methods of Aspects 35-52, wherein the eukaryotic cell further comprises a guide polynucleotide comprising a sequence substantially complementary to a sequence within the genome of said eukaryotic cell.

Aspect 55: Any of the methods of Aspects 35-52, further comprising providing to the eukaryotic cell a guide polynucleotide comprising a sequence substantially complementary to a sequence within the genome of said eukaryotic cell.

Aspect 56: Any of the methods of Aspects 35-52, wherein said eukaryotic cell comprises an endonuclease.

Aspect 57: The method of Aspect 20, wherein said endonuclease is Cas3.

Aspect 58: Any of the methods of Aspects 35-52, further comprising providing to the eukaryotic cell an endonuclease.

Aspect 59: The method of Aspect 58, wherein said endonuclease is Cas3.

Aspect 60: The method of any of Aspects 56-59, further comprising nicking or cleaving at least one strand of said polynucleotide target sequence.

Aspect 61: Any of the methods of Aspects 35-60, wherein said eukaryotic cell is a plant cell.

Aspect 62: The method of Aspect 61, wherein said plant cell is a monocot plant cell.

Aspect 63: The method of Aspect 62, wherein said monocot plant cell is obtained or derived from a plant selected from the group consisting of: corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

Aspect 64: The method of Aspect 61, wherein said plant cell is a dicot plant cell.

Aspect 65: The method of Aspect 64, wherein said dicot plant cell is obtained or derived from a plant selected from the group consisting of: soybean (Glycine max), Brassica species (for example but not limited to: oilseed rape or Canola) (Brassica napus, B. campestris, Brassica rapa, Brassica juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum).

Aspect 66: A eukaryotic cell produced by any of the methods of Aspects 35-65.

Aspect 67: An organism or tissue derived from the eukaryotic cell of Aspect 66.

Aspect 68: A progeny of the organism of Aspect 67.

Aspect 69: A plant derived from the eukaryotic cell of Aspect 66.

Aspect 70: A progeny of the plant of Aspect 69.

Aspect 71: Any of the methods of Aspects 37-70, wherein said cascade is a Type I-E CRISPR cascade.

Aspect 72: Any of the methods of Aspects 37-70, wherein said Cse1 polypeptide or cse1 polynucleotide was derived from a host organism comprising a Type I-E CRISPR system.

Aspect 73: A method for forming a CRISPR cascade and guide polynucleotide complex in a eukaryotic cell, comprising providing to said eukaryotic cell a guide polynucleotide comprising a variable targeting domain that is substantially complementary to a polynucleotide target sequence in the genome of said eukaryotic cell; wherein said eukaryotic cell comprises a cascade wherein one of the components is encoded by a cse1 polynucleotide; and incubating said cell; wherein the guide polynucleotide and cascade form a complex capable of recognizing and binding to said polynucleotide target sequence in the genome of said eukaryotic cell.

Aspect 74: A method for forming a CRISPR cascade and guide polynucleotide complex in a eukaryotic cell, comprising providing to said eukaryotic cell a guide polynucleotide comprising a variable targeting domain that is substantially complementary to a polynucleotide target sequence in the genome of said eukaryotic cell; wherein said eukaryotic cell comprises a cascade comprising a Cse1 polypeptide; and incubating said cell; wherein the guide polynucleotide and cascade form a complex capable of recognizing and binding to said polynucleotide target sequence in the genome of said eukaryotic cell.

Aspect 75: The method of Aspect 71 or 74, wherein said cascade further comprises a protein selected from the group consisting of: Cse2, Cas7, Cas5, and Cas6.

Aspect 76: The method of Aspect 71 or 74, wherein said cascade further comprises at least two proteins selected from the group consisting of: Cse2, Cas7, Cas5, and Cas6.

Aspect 77: The method of Aspect 71 or 74, wherein said cascade further comprises at least three proteins selected from the group consisting of: Cse2, Cas7, Cas5, and Cas6.

Aspect 78: The method of Aspect 71 or 74, wherein said cascade further comprises Cse2, Cas7, Cas5, and Cas6.

Aspect 79: The method of any of Aspects 75-78, wherein Cas5 is linked to a heterologous polypeptide, wherein said heterologous polypeptide is capable of a function selected from the group consisting of: transcription repression, transcription modulation, transcription activation, polynucleotide methylation, polynucleotide demethylation, polynucleotide cleavage, polynucleotide nicking, deamination, and a combination thereof.

Aspect 80: The method of Aspect 79, wherein the heterologous polynucleotide is linked to the C-terminal end of Cas5.

Aspect 81: The method of Aspect 79, wherein the Cas5 and the heterologous polynucleotide are linked by 10 amino acids.

Aspect 82: The method of Aspect 74, wherein a polynucleotide selected from the group consisting of: cse2, cas7, cas5, and cas6 is further introduced into said eukaryotic cell.

Aspect 83: The method of Aspect 74, wherein at least two polynucleotides selected from the group consisting of: cse2, cas7, cas5, and cas6 are further introduced into said eukaryotic cell.

Aspect 84: The method of Aspect 74, wherein at least three polynucleotides selected from the group consisting of: cse2, cas7, cas5, and cas6 are further introduced into said eukaryotic cell.

Aspect 85: The method of Aspect 74, wherein cse2, cas7, cas5, and cas6 are further introduced into said eukaryotic cell.

Aspect 86: The method of any of Aspects 74, 82, 83, 84, or 85, wherein at least one of the components is introduced into said eukaryotic cell via a recombinant DNA construct.

Aspect 87: The method of any of Aspects 74, 82, 83, 84, or 85, wherein at least one of the components has been optimized for expression in said eukaryotic cell.

Aspect 88: The method of Aspect 86, wherein the introduction into said eukaryotic cell is achieved via a method selected from the group consisting of: Agrobacterium-mediated transformation, particle bombardment, and floral dip.

Aspect 89: Any of the methods of Aspects 71-88, further comprising incubating said eukaryotic cell at a temperature of greater than 28 degrees Celsius.

Aspect 90: Any of the methods of Aspects 71-88, wherein the eukaryotic cell further comprises a guide polynucleotide comprising a sequence substantially complementary to a sequence within the genome of said eukaryotic cell.

Aspect 91: Any of the methods of Aspects 71-88, further comprising providing to the eukaryotic cell a guide polynucleotide comprising a sequence substantially complementary to a sequence within the genome of said eukaryotic cell.

Aspect 92: Any of the methods of Aspects 71-88, wherein said eukaryotic cell comprises an endonuclease.

Aspect 93: The method of Aspect 92, wherein said endonuclease is Cas3.

Aspect 94: Any of the methods of Aspects 71-88, further comprising providing to the eukaryotic cell an endonuclease.

Aspect 95: The method of Aspect 94, wherein said endonuclease is Cas3.

Aspect 96: The method of any of Aspects 92-95, further comprising nicking or cleaving at least one strand of said polynucleotide target sequence.

Aspect 97: Any of the methods of Aspects 71-96, wherein said eukaryotic cell is a plant cell.

Aspect 98: The method of Aspect 97, wherein said plant cell is a monocot plant cell.

Aspect 99: The method of Aspect 98, wherein said monocot plant cell is obtained or derived from a plant selected from the group consisting of: corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

Aspect 100: The method of Aspect 97, wherein said plant cell is a dicot plant cell.

Aspect 101: The method of Aspect 100, wherein said dicot plant cell is obtained or derived from a plant selected from the group consisting of: soybean (Glycine max), Brassica species (for example but not limited to: oilseed rape or Canola) (Brassica napus, B. campestris, Brassica rapa, Brassica juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum).

Aspect 102: A eukaryotic cell produced by any of the methods of Aspects 71-101.

Aspect 103: An organism or tissue derived from the eukaryotic cell of Aspect 102.

Aspect 104: A progeny of the organism of Aspect 103.

Aspect 105: A plant derived from the eukaryotic cell of Aspect 102.

Aspect 106: A progeny of the plant of Aspect 105.

Aspect 107: Any of the methods of Aspects 72-106, wherein said CRISPR cascade is a Type I-E cascade.

Aspect 108: Any of the methods of Aspects 72-106, wherein said CseI polypeptide or cse1 polynucleotide was derived from a host organism comprising a Type I-E CRISPR system.

Aspect 109: A method for providing a trait of agronomic importance to a plant, comprising: (a) providing to a plant cell a Cse1 polypeptide, an endonuclease, and a guide polynucleotide comprising a variable targeting domain that is substantially complementary to a polynucleotide target sequence in the genome of the plant cell, (b) placing the plant cell in a medium that promotes growth, (c) generating a plant from the plant cell, and (d) screening said plant for a trait of agronomic importance; wherein said Cse1 polypeptide functionally associates with at least one other protein to form a cascade; and wherein the guide polynucleotide and cascade form a complex capable of recognizing and binding to the polynucleotide target sequence in the genome of the plant cell.

Aspect 110: A method for providing a trait of agronomic importance to a plant, comprising: (a) providing to a plant cell a cse1 polynucleotide, an endonuclease, and a guide polynucleotide comprising a variable targeting domain that is substantially complementary to a polynucleotide target sequence in the genome of the plant cell, (b) placing the plant cell in a medium that promotes growth, (c) generating a plant from the plant cell, and (d) screening said plant for a trait of agronomic importance; wherein said cse1 polynucleotide is operably linked to a noncoding regulatory element rendering it capable of expressing a Cse1 polypeptide, and wherein said Cse1 polypeptide functionally associates with at least one other protein to form a cascade; and wherein said cascade and guide polynucleotide form a complex capable of recognizing and binding to a polynucleotide target sequence in the genome of said eukaryotic cell.

Aspect 111: The method of Aspect 107 or 110, wherein said cascade further comprises a protein selected from the group consisting of: Cse2, Cas7, Cas5, and Cas6.

Aspect 112: The method of Aspect 107 or 110, wherein said cascade further comprises at least two proteins selected from the group consisting of: Cse2, Cas7, Cas5, and Cas6.

Aspect 113: The method of Aspect 107 or 110, wherein said cascade further comprises at least three proteins selected from the group consisting of: Cse2, Cas7, Cas5, and Cas6.

Aspect 114: The method of Aspect 107 or 110, wherein said cascade further comprises Cse2, Cas7, Cas5, and Cas6.

Aspect 115: The method of any of Aspects 111-114, wherein Cas5 is linked to a heterologous polypeptide, wherein said heterologous polypeptide is capable of a function selected from the group consisting of: transcription repression, transcription modulation, transcription activation, polynucleotide methylation, polynucleotide demethylation, polynucleotide cleavage, polynucleotide nicking, deamination, and a combination thereof.

Aspect 116: The method of Aspect 115, wherein the heterologous polynucleotide is linked to the C-terminal end of Cas5.

Aspect 117: The method of Aspect 115, wherein the Cas5 and the heterologous polynucleotide are linked by 10 amino acids.

Aspect 118: The method of Aspect 110, wherein a polynucleotide selected from the group consisting of: cse2, cas7, cas5, and cas6 is further introduced into said eukaryotic cell.

Aspect 119: The method of Aspect 110, wherein at least two polynucleotides selected from the group consisting of: cse2, cas7, cas5, and cas6 are further introduced into said eukaryotic cell.

Aspect 120: The method of Aspect 110, wherein at least three polynucleotides selected from the group consisting of: cse2, cas7, cas5, and cas6 are further introduced into said eukaryotic cell.

Aspect 121: The method of Aspect 110, wherein cse2, cas7, cas5, and cas6 are further introduced into said eukaryotic cell.

Aspect 122: The method of any of Aspects 110, 118, 119, 120, or 121, wherein at least one of the components is introduced into said eukaryotic cell via a recombinant DNA construct.

Aspect 123: The method of any of Aspects 110, 118, 119, 120, or 121, wherein at least one of the components has been optimized for expression in said eukaryotic cell.

Aspect 124: The method of Aspect 122, wherein the introduction into said eukaryotic cell is achieved via a method selected from the group consisting of: Agrobacterium-mediated transformation, particle bombardment, and floral dip.

Aspect 125: Any of the methods of Aspects 107-124, further comprising incubating said eukaryotic cell at a temperature of greater than 28 degrees Celsius.

Aspect 126: Any of the methods of Aspects 107-124, wherein the eukaryotic cell further comprises a guide polynucleotide comprising a sequence substantially complementary to a sequence within the genome of said eukaryotic cell.

Aspect 127: Any of the methods of Aspects 107-124, further comprising providing to the eukaryotic cell a guide polynucleotide comprising a sequence substantially complementary to a sequence within the genome of said eukaryotic cell.

Aspect 128: Any of the methods of Aspects 107-124, wherein said eukaryotic cell comprises an endonuclease.

Aspect 129: The method of Aspect 128, wherein said endonuclease is Cas3.

Aspect 130: Any of the methods of Aspects 94-106, further comprising providing to the eukaryotic cell an endonuclease.

Aspect 131: The method of Aspect 130, wherein said endonuclease is Cas3.

Aspect 132: The method of any of Aspects 128-131, further comprising nicking or cleaving at least one strand of said polynucleotide target sequence.

Aspect 133: Any of the methods of Aspects 107-132, wherein said eukaryotic cell is a plant cell.

Aspect 134: The method of Aspect 133, wherein said plant cell is a monocot plant cell.

Aspect 135: The method of Aspect 134, wherein said monocot plant cell is obtained or derived from a plant selected from the group consisting of: corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

Aspect 136: The method of Aspect 133, wherein said plant cell is a dicot plant cell.

Aspect 137: The method of Aspect 136, wherein said dicot plant cell is obtained or derived from a plant selected from the group consisting of: soybean (Glycine max), Brassica species (for example but not limited to: oilseed rape or Canola) (Brassica napus, B. campestris, Brassica rapa, Brassica juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum).

Aspect 138: The method of any of Aspects 107-137, wherein said trait of agronomic importance is selected from the group consisting of: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, and altered seed nutrient composition.

Aspect 139: A eukaryotic cell produced by any of the methods of Aspects 107-138.

Aspect 140: An organism or tissue derived from the eukaryotic cell of Aspect 139.

Aspect 141: A progeny of the organism of Aspect 140.

Aspect 142: A plant derived from the eukaryotic cell of Aspect 139.

Aspect 143: A progeny of the plant of Aspect 142.

Aspect 144: Any of the methods of Aspects 111-143, wherein said cascade is a Type I-E CRISPR cascade.

Aspect 145: Any of the methods of Aspects 111-143, wherein said Cse1 polypeptide or cse1 polynucleotide was derived from a host organism comprising a Type I-E CRISPR system.

Aspect 146: A synthetic composition comprising: (a) a eukaryotic cell, (b) a guide polynucleotide comprising a variable targeting domain that is substantially complementary to a polynucleotide target sequence in the genome of said eukaryotic cell, (c) an endonuclease, and (d) a cascade comprising a Cse1 polypeptide; wherein said cascade forms a complex with the guide polynucleotide and the endonuclease; and wherein the complex is capable of recognizing, binding to, and nicking or cleaving at least one strand of a polynucleotide target sequence in the genome of said eukaryotic cell.

Aspect 147: A synthetic composition comprising: (a) a eukaryotic cell, (b) a guide polynucleotide comprising a variable targeting domain that is substantially complementary to a polynucleotide target sequence in the genome of said eukaryotic cell, (c) an endonuclease, and (d) a cse1 polynucleotide; wherein said cse1 polynucleotide is operably linked to a noncoding regulatory element rendering it capable of expressing a Cse1 polypeptide that functionally associates with at least one other protein to form a cascade; wherein said cascade forms a complex with the guide polynucleotide and the endonuclease; and wherein the complex is capable of recognizing, binding to, and nicking or cleaving at least one strand of a polynucleotide target sequence in the genome of said eukaryotic cell.

Aspect 148: The synthetic composition of Aspect 144 or 147, wherein said cascade further comprises a protein selected from the group consisting of: Cse2, Cas7, Cas5, and Cas6.

Aspect 149: The synthetic composition of Aspect 144 or 147, wherein said cascade further comprises at least two proteins selected from the group consisting of: Cse2, Cas7, Cas5, and Cas6.

Aspect 150: The synthetic composition of Aspect 144 or 147, wherein said cascade further comprises at least three proteins selected from the group consisting of: Cse2, Cas7, Cas5, and Cas6.

Aspect 151: The synthetic composition of Aspect 144 or 147, wherein said cascade further comprises Cse2, Cas7, Cas5, and Cas6.

Aspect 152: The synthetic composition of any of Aspects 148-151, wherein Cas5 is linked to a heterologous polypeptide, wherein said heterologous polypeptide is capable of a function selected from the group consisting of: transcription repression, transcription modulation, transcription activation, polynucleotide methylation, polynucleotide demethylation, polynucleotide cleavage, polynucleotide nicking, deamination, and a combination thereof.

Aspect 153: The method of Aspect 152, wherein the heterologous polynucleotide is linked to the C-terminal end of Cas5.

Aspect 154: The method of Aspect 152, wherein the Cas5 and the heterologous polynucleotide are linked by 10 amino acids.

Aspect 155: The synthetic composition of Aspect 147, further comprising a polynucleotide selected from the group consisting of: cse2, cas7, cas5, and cas6.

Aspect 156: The synthetic composition of Aspect 147, further comprising at least two polynucleotides selected from the group consisting of: cse2, cas7, cas5, and cas6.

Aspect 157: The synthetic composition of Aspect 147, further comprising at least three polynucleotides selected from the group consisting of: cse2, cas7, cas5, and cas6.

Aspect 158: The synthetic composition of Aspect 147, further comprising cse2, cas7, cas5, and cas6.

Aspect 159: The synthetic composition of any of Aspects 147, 155, 156, 157, or 158, wherein at least one of the components were introduced into said eukaryotic cell via a recombinant DNA construct.

Aspect 160: The synthetic composition of any of Aspects 147, 155, 156, 157, or 158, wherein at least one of the components has been optimized for expression in said eukaryotic cell.

Aspect 161: The synthetic composition of Aspect 159, wherein the introduction into said eukaryotic cell is achieved via a method selected from the group consisting of: Agrobacterium-mediated transformation, particle bombardment, and floral dip.

Aspect 162: Any of the synthetic compositions of Aspects 144-161, wherein said eukaryotic cell is incubated at a temperature of greater than 28 degrees Celsius.

Aspect 163: Any of the synthetic compositions of Aspects 144-161, wherein the eukaryotic cell further comprises a guide polynucleotide comprising a sequence substantially complementary to a sequence within the genome of said eukaryotic cell.

Aspect 164: Any of the synthetic compositions of Aspects 144-161, wherein said eukaryotic cell further comprises an endonuclease.

Aspect 165: The synthetic composition of Aspect 164, wherein said endonuclease is Cas3.

Aspect 166: Any of the synthetic compositions of Aspects 144-161, further comprising an endonuclease.

Aspect 167: The synthetic composition of Aspect 166, wherein said endonuclease is Cas3.

Aspect 168: Any of the synthetic compositions of Aspects 144-161, wherein said eukaryotic cell is a plant cell.

Aspect 169: The synthetic composition of Aspect 168, wherein said plant cell is a monocot plant cell.

Aspect 170: The synthetic composition of Aspect 169, wherein said monocot plant cell is obtained or derived from a plant selected from the group consisting of: corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

Aspect 171: The synthetic composition of Aspect 168, wherein said plant cell is a dicot plant cell.

Aspect 172: The synthetic composition of Aspect 171, wherein said dicot plant cell is obtained or derived from a plant selected from the group consisting of: soybean (Glycine max), Brassica species (for example but not limited to: oilseed rape or Canola) (Brassica napus, B. campestris, Brassica rapa, Brassica juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum).

Aspect 173: A eukaryotic cell produced by any of the synthetic compositions of Aspects 144-172.

Aspect 174: An organism or tissue derived from the eukaryotic cell of Aspect 173.

Aspect 175: A progeny of the organism of Aspect 174.

Aspect 176: A plant derived from the eukaryotic cell of Aspect 173.

Aspect 177: A progeny of the plant of Aspect 176.

Aspect 178: Any of the synthetic compositions of Aspects 136-177, wherein said cascade is a Type I-E CRISPR cascade.

Aspect 179: Any of the synthetic of Aspects 136-177, wherein said Cse1 polypeptide or cse1 polynucleotide was derived from a host organism comprising a Type I-E CRISPR system.

Aspect 180: A synthetic composition comprising a Cas5 protein and a heterologous polypeptide, wherein said heterologous polypeptide is capable of a function selected from the group consisting of: transcription repression, transcription modulation, transcription activation, polynucleotide methylation, polynucleotide demethylation, polynucleotide cleavage, polynucleotide nicking, deamination, and a combination thereof

Aspect 181: The synthetic composition of Aspect 178, wherein said Cas5 protein and said heterologous polynucleotide are linked by a sequence of 10 amino acids.

We claim:

1. A method for modifying a polynucleotide sequence of a target site in the genome of a plant cell, comprising providing to the plant cell the following molecules:
(a) a Cascade comprising at least one component selected from the group consisting of: Cse1, Cse2, Cas5, Cash, and Cas7;
(b) a molecule capable of nicking, cleaving, or editing at least one nucleotide of the polynucleotide sequence of the target site; and
(c) a guide polynucleotide comprising a variable targeting domain that is substantially complementary to the polynucleotide target sequence in the genome of said eukaryotic cell; and
wherein the guide polynucleotide and Cascade form a complex capable of recognizing and binding to the polynucleotide sequence of the target site; and wherein the polynucleotide sequence of the target site is modified by the addition at least one nucleotide, the substitution of at least one nucleotide, the deletion of at least one nucleotide, the chemical alteration of at least one nucleotide, or any combination of the preceding.

2. The method of claim 1, wherein the molecule of (b) is a Cas3 nuclease.

3. The method of claim 1, wherein the molecule of (b) is Fok1.

4. The method of claim 1, wherein the molecule of (b) is I-TevI.

5. The method of claim 1, wherein the molecule of (b) is an engineered Cas3 nickase.

6. The method of claim 5, wherein nicking occurs on both strands of a double-stranded polynucleotide to create a double-strand break.

7. The method of claim 1, wherein at least one of the components of (a) is a fusion protein further comprising a transcriptional activator.

8. The method of claim 1, wherein a plurality of components of (a) each further comprise a transcriptional activator.

9. The method of claim 1, wherein the molecule of (b) is a nuclease domain that is fused to at least one of the components of (a).

10. The method of claim 9, wherein the nuclease domain is I-TevI.

11. The method of claim 9, wherein the nuclease domain is Fok1.

12. The method of claim 1, wherein the target sequence is an endogenous gene of the cell.

13. The method of claim 1, wherein the target sequence is heterologous to the cell.

14. The method of claim 1, further comprising providing to the cell a heterologous polynucleotide.

15. The method of claim 14, wherein the heterologous polynucleotide is a DNA repair template, and wherein the polynucleotide sequence of the target site is modified to include at least one base pair alteration as compared to its native state.

16. The method of claim 14, wherein the heterologous polynucleotide is a donor DNA molecule, and wherein the polynucleotide sequence of the target site is modified by the incorporation of the heterologous polynucleotide.

17. The method of claim 1, wherein at least one component of the Cascade in (a) is provided as a polynucleotide sequence in a recombinant construct encoding the polypeptide component.

18. The method of claim 1, wherein at least one component of the Cascade in (a) is provided as a polypeptide.

19. The method of claim 1, wherein the components of the Cascade in (a) and the molecule of (b) are provided as polynucleotides in one or more recombinant constructs.

20. The method of claim 1, wherein the guide polynucleotide of (c) is provided as a DNA molecule that is operably linked to a heterologous regulatory element.

21. The method of claim 20, wherein the heterologous regulatory element is a polII promoter or a polIII promoter.

22. The method of claim 1, wherein the guide polynucleotide of (c) is provided as a polyribonucleotide molecule, optionally further comprising at least one deoxyribonucleotide.

23. The method of claim 1, wherein at least one of the molecules provided to the cell is provided via Agrobacterium-mediated transformation.

24. The method of claim 1, wherein at least one of the molecules provided to the cell is provided via particle bombardment.

25. The method of claim 1, wherein the modification of the polynucleotide sequence of the target site results in transcriptional activation of a gene.

26. The method of claim 1, further comprising placing the plant cell in a medium that promotes viability, and screening the cell for the presence or absence of a trait of interest.

27. The method of claim 1, further comprising placing the plant cell in a medium that promotes growth, generating a plant from the plant cell, and screening the plant for the presence or absence of a trait of agronomic interest.

28. The method of claim 1, wherein the plant cell is obtained or derived from a plant selected from the group consisting of: corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrass, other grass, soybean (Glycine max), Brassica species (for example but not limited to: oilseed rape or Canola) (Brassica napus, B. campestris, Brassica rapa, Brassica juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), and potato (Solanum tuberosum).

29. A synthetic composition comprising:
(a) a plant cell;
(b) Cse1, Cse2, Cas5, Cash, Cas7;
(c) a molecule capable of nicking or cleaving a polynucleotide sequence of a target site in the plant cell; and
(d) a guide polynucleotide comprising a variable targeting domain that is substantially complementary to a polynucleotide target sequence in the plant cell.

30. The method of claim 29, wherein the molecule of 29(c) is a Cas3 nuclease,

31. The method of claim 29, wherein the molecule of 29(c) is Fok1.

32. The method of claim 29, wherein the molecule of 29(c) is I-TevI.

33. The method of claim 29, wherein the molecule of 29(c) is an engineered Cas3 nickase.

34. The method of claim 29, wherein at least one of the components of 29(b) is a fusion protein further comprising a transcriptional activator.

35. The method of claim 29, wherein a plurality of components of 29(b) each further comprise a transcriptional activator.

36. The method of claim 29, wherein the molecule of 29(c) is a nuclease domain that is fused to at least one of the components of 29(b).

37. The synthetic composition of claim 29, wherein at least one of the members of 29(b) further comprises a heterologous nuclease domain or a transcriptional activator.

38. The method of claim 29 further comprising a heterologous polynucleotide.

39. The method of claim 38, wherein the heterologous polynucleotide is a DNA repair template, and wherein the polynucleotide sequence of the target site is modified to include at least one base pair alteration as compared to its native state.

40. The method of claim 38, wherein the heterologous polynucleotide is a donor DNA molecule, and wherein the polynucleotide sequence of the target site is modified by the incorporation of the heterologous polynucleotide.

41. The synthetic composition of claim 29, wherein the plant cell is obtained or derived from a plant selected from the group consisting of: corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, other grass, soybean (Glycine max), Brassica species (for example but not limited to: oilseed rape or Canola) (Brassica napus, B. campestris, Brassica rapa, Brassica juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), and potato (Solanum tuberosum).

42. A method for modifying a polynucleotide sequence of a target site in the genome of a plant cell, comprising providing to the plant cell the following molecules:
(a) a Cascade comprising Cse1, Cse2, Cas5, Cas6, and Cas7;
(b) a Cas3 nickase; and
(c) a guide polynucleotide comprising a variable targeting domain that is substantially complementary to said polynucleotide target sequence in the genome of said plant cell; and
wherein the guide polynucleotide and Cascade form a complex capable of recognizing and binding to the polynucleotide sequence of the target site; and wherein the polynucleotide sequence of the target site is modified by the addition at least one nucleotide, the substitution of at least one nucleotide, the deletion of at least one nucleotide, the chemical alteration of at least one nucleotide, or any combination of the preceding.

43. A method for modifying a polynucleotide sequence of a target site in the genome of a plant cell, comprising providing to the plant cell the following molecules:
(d) a Cascade comprising Cse1, Cse2, Cas5, Cas6, and Cas7;
(e) I-TeVI; and
(f) a guide polynucleotide comprising a variable targeting domain that is substantially complementary to said polynucleotide target sequence in the genome of said plant cell; and
wherein the guide polynucleotide and Cascade form a complex capable of recognizing and binding to the polynucleotide sequence of the target site; and wherein the polynucleotide sequence of the target site is modified by the addition at least one nucleotide, the substitution of at least one nucleotide, the deletion of at least one nucleotide, the chemical alteration of at least one nucleotide, or any combination of the preceding.

44. A method for modifying a polynucleotide sequence of a target site in the genome of a plant cell, comprising providing to the plant cell the following molecules:
(g) Cascade genes cse1, cse2, cas5, cas6, and cas7;
(h) a gene encoding a Cas3 nickase; and
(i) a guide polynucleotide comprising a variable targeting domain that is substantially complementary to said polynucleotide target sequence in the genome of said plant cell; and
wherein the guide polynucleotide and Cascade form a complex capable of recognizing and binding to the polynucleotide sequence of the target site; and wherein the polynucleotide sequence of the target site is modified by the addition at least one nucleotide, the substitution of at least one nucleotide, the deletion of at least one nucleotide, the chemical alteration of at least one nucleotide, or any combination of the preceding.

45. A method for modifying a polynucleotide sequence of a target site in the genome of a plant cell, comprising providing to the plant cell the following molecules:
Cascade genes cse1, cse2, cas5, cas6, and cas7;
(k) a gene encoding I-TevI; and
(l) a guide polynucleotide comprising a variable targeting domain that is substantially complementary to said polynucleotide target sequence in the genome of said plant cell; and
wherein the guide polynucleotide and Cascade form a complex capable of recognizing and binding to the polynucleotide sequence of the target site; and wherein the polynucleotide sequence of the target site is modified by the addition at least one nucleotide, the substitution of at least one nucleotide, the deletion of at least one nucleotide, the chemical alteration of at least one nucleotide, or any combination of the preceding.
